SEQUENCE LISTING (30 sequences)

SEQ ID NO: 1 - DNA, 3813 bp, Phytophthora ramourum
atggcttcagcttggcgccagcatccgcacgatccgtgggacctgccgtcgccgccgccgctactagaagaagccgcgcccaaagcttccaagcgctcacacttttcacgccgcagcacgcgcgcccagagccaagcgagagcccaagcgcagcaggcggagctcgaggtggaggcgctcgccgaccagcgcgaccgcgacgagcgccgcagcctcctggccgccatgttcggtggtcgcagcaagtcacgcacgtcgcgatcgtcctcggacgccccgcagacgctcgtgcagcgagaaaaggccttccgcctcgtaggcaacttcgtcaagatgggctggctcaccaagcagggccacatgtggaagagctggaagacgcgcttttttgtgctcttctcggacggaacgttcgcctactacaagaacaagggccgcaagaaaatcaagggctgcatgcagctcaatgacggcgtcgtgtccgtgcaacacgtggacgtccgcgtggcggacaaggcctacgtattccagatagagaaggggttttacaagctgctgtgttattgctgcagccagtttgaggccgagctgtgggtcgcagcgctgcgctcggtgcggagagtgaccccgccctgttacgagacggatctaacggccactgaggagaaggcgggctccaacgcggtcacgagacacttgaacaagattttcatcacagataagcagatcgccaagagtttggtgcagttcaaggtgaacgagcacgaccattcgtacgcagcaattcacaattttattgtcgagctggacgacgcgatcattgatggtcatcatttggagctctatcaggacccagagattgagctgctgcctggtaatgacctggtacggctgattcgtcgacatgtggaagatcgagtgtttattccgttgtatgcggaggcgtacgcgagcttggagacggacaaggtcaagacgaggcgcagcaacctcgagcagaacctcaaggtgctgaggcggaaaactcaggcagattttggcatctcgaaggatttatccgtttgcgactggaagctagccatcaatgtgattaacatgctcgaatgcgtgtcgctcccaacgcacaagttcgaggtcattttatcagctggaaaggccatcacggagtctattgctcaatataacggcgacctgttcgaggtatcagatgagacgctgacggcggtttttcggtacgtagttacaatggcgtcactgagcgatttaccgattctgcgagcgcttctcaagtacggataccagcaccacccagcgtgtcagaacaaagcaaacgtcgtttcagctttcctcgatgggatcaaatgggtggagtgcttcgagactggcgatgaaagctatcagtttgattccttagcgctagcagggtctcgcgtgtcggtatccatctcgactaacgatgtcggcattcagtttacgaccgatggcaatggtagaggggctattgtttacagtgtgcgaaagcaatcacaagcagcacttagcgcttcaatcgtccctggattgtcgctgattgccatcaaccacgaaccagttatcgggatgcctttcgacaagattatccagcgcgtgcgaactgcggcactccctaagcaactcattttcatgaccgagttttactactaccagcttctttcgctggattcggagatgtttcgatacctaatgtgtattgcagcgcgccgtggggacttggattcggctgcttggctgcggtcgtccacggtggagctcaataaactttgctcgtgggaaaaatctcgaggaaaacaggtctttggattcatccctgtatctgggaagggatccccacttcacgcggcagtgcataacggtcaactctctatggtgaagtatcttatctcaagaggggctgatacgaacttgtgcaatcagaaaggaagacgcccacttcatgtcgtgaagcagtcgattgatatggcgttgatcattgagagccttattgatgcgggagctgacattgacgtagcggaaaaacacggtcttaccccgctaatgttcatgtgctttagagcatcgcttgaaggtgccgcgacgcttgtagcacttggagcagatgttcgccgtgttgcatggtcgaatggattttcggctctggagtttgcagtgaagagtgaacgaacagagctggtagagttgtgcctttcaaagggggcaaatccaaatatgcctacgctggacggaaacacaagtttgcatatggcagccgctctcccccacgctgatataatcctccggctcctacagagcggtgcaaaccccaatgttcagaatcggtatggtcagactccggccacagtcctacttgcgtctgctcctggtggaggtggcgatgcccgaatcctgtgtctggagattttaacatgtgccggttgccggttggacaaacgtgatctctttggtcgccaggcttctcacctggcaaacatctcgcgagattctcatgtcatgggtctacttcgaaagttgggatcgttgaaccgagcaggcaatagcgttgatgtcgatatcttcggttgctcacctgttgattatagtcatgggatcgaggtaagcaaggcgacagaatattttggatcgcaaatgccgaacaattcgtgggaaatcattgacaagaccgaccacgtgtctcgatctgcactctcgggttctgtcgaggatttgattcgcaagttagttgcaggcacggaagtggacttggtggatgtggtggcgttcgtgctcttcttggacagcttctcgagtcttaacgaattggtggatcggttgagcgagcatgtccgaaacgggattaaaagccacggacttatccgtctcttcatcatgatcctgctttttagaaatacggaaacgaaggaagtagatgatctgtctgatcgcttctacagcctgatcagtcacaacattgacgtccctagggaaaagttggcattgagttcgtatccgacgagccctttccgcttcatttgcaccgaacggtgggctgaacagtgcacgcttctgacgcacgcagtcttttgccagattccagtccaagatttgattttgacaggctcgaaaaagcagcactccgttgaatttacgagtatcaaacgatggtttcagcacatctctgcgtacgtgatcaacgctgttcttgtacaaaactccccagaagagcgtgccgaagtaatttcgttttatctcaagctaagtgcggaagcgaagaagatgatgaacgaaatgcagctactttccgacaaaggatgtcgcgagatgaaccggttgatgcgcaaaactgtcaaccccagtatgccttatattgggttgtatctccagggcttcgtcggactgaacgagttgcctgcgtttggcaaagagggtcaagtcaatgcgagtcggctacgaaaaatgggcgagttggcaatggaaatcttgcaccgccagtctgttgcgtacactcttcaaaacgacgagagtattgataaactgttgcacgtttcaatgccgtattcgtcggaggagagtcgatatgctcggtcattggaggtcgagccgcgtgaagcagatgcgattccattgagcgatcgcggttcgtgtgttgttgttgatgacgacctcgaccttgaaaatgaagttcgcgagtcagttggcggcgatggaacctttggattccgccagtggattaggaagcagcaagtcgtgcaccgcaatcgctcacgctcttcgctgatagctttgtacgagtgggtgtag

SEQ ID NO: 2 - DNA, 3852 bp, Phytophthora sojae
atggcttcaggctggcggcagaaaccgcgcctcgatccgtgggacccgccgtcgcccccgccgctgctggaggaggccgcgcccaagcccaagcccaaaaccagcaggttctcgcgccgcagcacccgcgccaagagccacgcgcgccagcaggcgcagcacgtggagctggagcgcgagaaggccttccgcctggtgcgccacggctccgacccgctgctgaacgacgcgttctacgccacgcaggtgcaggtgggcaacttcgtcaagatggggtggctcaccaagcagggccacatgtggaagagctggaagacgcgctttttcgtgctcttctcggacggcaccttcgcctactacaagaacaagggccgcaagaagatcaagggctgcatgcagctcaacgatggcgtcgtgtccgtgcagcacgtggacatccgcctggccgacaaggcctacgtgttccagatcgagaagggcttctacaagctgctgtgctactgctgcagccagttcgaggcggagctgtgggtcgctgcactgaggtcggtgcgcagagtggcaccgccgtgctatgagatggacctgacggccactgaggagaaggccgggtccaatgcggtcaccaggcacttgaacaagatcttcatcacggacaagcagatcgccaagaggctggcggagttcaaggccaacacacatgaccactcgtacgcggccattcacaatttcatagtggagctggacgacgcgatcattgatcgtcaccatttggagttttatcaggacgcggagatcgagctcttgccgggcaacgagctggtgcggctgattcggcggcatgtggaagaccgcgtgttcattccgctgtatgcagaggcgtatgcgagcttggaaacggacaaggtgaaaacgaggcgcagcaacctcgagcagcatctcaaggtactgaagcagaaaacgcaggccgatttcggcatctcgaaggacctttcagtttgcaattggaagcaggctatcagcgttgttaacatgctggattgcgtgtcactacctacgcacaagtttgaagtcatcctatcagctggaaaagcgatcatggagctgatcgctcaatacaatggcgaacttttcgaagtgtcagacgagatgttaacggcgatcttccggtacgtggtaacgatgtcttcgctgagcgatctaccaactcttcgagcgctgctgaagtacggctatcagcatcatccagcatcgcaaaacaaagcgaatgtggttacggcctttctcaacgccatcaagtgggtggagtgtttcgaggccggcgacgagagttaccagttcgattcgttggcgctggctggatctcgagtttctgtatccatctcaaccaatgatgtcggcattcaattcactacggatggtaacgggcgaggagcaatcgtgtacaatatccgaaagcagtctcaggcagcgctgagcgctgcgattgtgcctggtctgtcattgatcgcgatcaaccacgagcccgtcattggtatgcctttcgacaagattattcaacgagtgcgcaccgcagcgctcccaaagcaactcactttcatgacggagttttattactaccaactactctcgctagactcggagatgtatcagtacctgatgtgtattgcagctcgacgtggtgacttggactctgctgcctggatgcgatcctcaacgattgaactcaatacgctatgctcgtgggagaagtctcgtggaaaacaggtttttgggttcacacctgtgtctggaaaaggatcgccacttcatgcagcagtacataacggacagcttagcatggtgaactaccttatttcaagaggagcagacgttaatctgtgcaaccagaagggacggcgcccactgcacgttgtgaagcagtcaatcgacatggccatgattattcaaagcttgatcgatgccggggctgatatcgacgcgatggagaaacacgggcttaccccgttaatgttcatgtgctccagagcatctctcgaaggatcagcaacactcctagcactcggtgcagatgttcactgtgttgcgtggacaaacggattttcagcactggagtttgcggtgaaaagtgagcataccgaattggtcgagctgtgtctctccaagggggcgaatccgaatgccccaacgttggatgggaacaccagtctacatttggccgccactcaggccaacactgatatcattcttcgacttttgcaaggtggagcaaaccccaacgttcaaaatcggtacggacaaacgccggcagcactattgcttgcgtcttcacccggcggaagtagcgacgtacgcatcctatgcctcgaaattttgacgtgtgctggctgccgccttgacaaaagagatctatttggccgccaggcttctcatctcgccagtatttcaagagactctcacgttgtcgatctacttcgcaagttgggctcgctgaatcgggaagccaatggtgttgaaattgacatcttcggttgttcttcagtcgactatagtcaccgtattgaggtggacaagtctgcaagctatctggggtctcaaatgcccaacagttcgtggaaaatggtggacaaggacgaccatgtgtctcgatctgcacgcccgggctcggttgatgatctgatccgcgagttagtggcagggacggaagtggacttggtggatgtggtgtctttcgtgctatttctcgatagcttttcgagcttgaacgaagtagtggatcgcctgagcgcacacgtccgaaacggtgttaaaagccacggacttatccgtctctttattgtggttctgctcttcaggcaatcggaaaccaaggaagctgacgatctgcgtgatcgtttctacagcctcattagtcttaacattgacgcgaaggacaaactggagtatttctccttgcgaggagctggaaattacgcggtgaactaccaagcgattccaggaatgatgagcaaggtttacggaatggagtcaggcttcgagcccgtatcctctggacccttttcggctcatttgcatcggttcgtcgacgcagaacggtgggcggagcagtgcaccctcttaacgcacgcagtgttctgcaagattccggtccgagacttcattccaactggatcgaagaaaaagcactcggttgagtttacgagcattaaacgctggttccagcacatctctgcatttgtgatcaatgctgtgctggttcagaataccccggaagaacgtgccgaagtgatttcgttttatctcaagctaagtctggacgcgaagaaaatgatgaacgagatgcagctactttcagacaagggttgccgagagatgaatagactgatgcgcaaaaccgctaatccgagcatgccatacatcggactgtacctccagggctttgtcggactcaacgagttgcctgcttttgacaaagagggcctggtgaacgcgaatcgtctacgaaggatgggcgagctggccatggaaatcctgcaccgccagtctgttgcctacacgcttcagcacgacgaagatgttaacaaactgctgcacgtttccctgccgtactcttctgaagagagtcggtatgctcggtcattggagttagagccgcgtgaagctgacgcgatcccattgagcgatcgtggttcgtatactttgatcgatgacgatgttgacctcgaaaatgaagttcgtgagtcaatcggcggtgacgggactttcggcttccgtcagtggatccggaagcagcaggtagtgcatcgaaaccgttcgcggtcgtcggtgatagctctgtacgagtgggtgtag

SEQ ID NO: 3 - DNA, 4044 bp, Phytophthora infestans
gttacccaagcgatggcttccggctggcgccacaaaggcaccaaagccgtcctggatccgtgggggtctccatcgcctcctccactactcgacgagacggctcctaagatcagcaagcgctcccgcttctcacgtcgcagcacacacgccaaaagccaagcgcaacaccaagctcagaacatcgagctcgaagttgaagaatttgccgaacaacaggaccgtgacaaacgtcgcagcctcttagcctccatgttcagaacccgagcactgtcgcgctcatcgcgctccatatcggacgccacggaggcgccacaggcgctcgtccagcgagataaaggtttccgactggtgcgtcacggctccgatccactcctaaacgactcgttttacgccactcaagtgcaggttggcaatttcgtcaagatgggctggctcaccaaacaaggacacatgtggaagagctggaagacgcgcttcttcgtgcttttctcggatggcacttttgcctactacaagaacaagggcaggaagaagatcaaaggttgcatgcaactaaacgacggcgtggtttccgtgcagcacgtggacatccgagtagctggtaaagcgtatgtgttccagatcgagaaaggtttctacaaattattgtgctattgctgcagccagtttgaagccgagttgtgggtcgctgcattgcggtcagtgaggagggtatctccaccctgttatgaaatggacctgactgcgaccgaagagaaagcaggatccaatgcagttaccagactcttgaacaagatcttcatcacggacaaagaggtggctaaaaactgggtccagttcaaggaaaatgatcacgaccattcgtacgctgcaatccacaatttcatcgtggaattggacgactctatcatcgatcgtcaccatttggagctctatcaagatccagagattgaattattgccaggtaatgagctcgtgcgactgattcggcgacatgtcgaggaccgtgtgtttattccgttgtatgcggaggcatacgcgagcttagagacggataaagtcaagtcgagacggagcaacttggagcagaacgttaaggtgttgaaacagaaagcacaggcggatttcggcatctcgaaagacttgtcggtttgcaattggaagcaagctatcagtgtgatcaatatgctcgactctgtgtcgctgccgacgcacaaattcgaggtcattttgtccgccgggaagaccgtcacagaggagattgctcagtataatggcgagctcttcgaggtcacagacgagacgttgacggcggtgttccggtacgtggtaacgatgtcctcgctcagcgacttagcgattcttcgagcactactgaagtatggatatcagcaccacccagcatgtcagaataaagcgaatgtcgtgactgcttttctcgatgcgatcaaatgggttgagaactttgagtctggagatgaaagctaccgattcgacacgatggcgctggcaggatctcgtgtctccgtatccatttcgaccaacgatattgggattcagctcacgacagacggaaatggaagaggagctattgtttatagtgttcggaagcagtctcaagcagctttgagcgctgccatcgtgcctggattgtcgctgatcgcgattaaccgtgagcctgtgattggaatgcctttcgagaaaattatccaacgcgttcggactgcagcattgccgaagcaacttacgttcatgacggagttttactattatcagctgttgtcactggacttggagatgtcccgctatttgacgtgcattgcagctcgtcgtggagacttggactcggctggatggctgcgatcctcgactgtggaattgaacactctttgctcgtgggaaaagactcgaggcaagcaggttttcggattcattccagcatccggcaagggctcgccgcttcatgctgctgtgcacaatggacaactcggaatggttaattatcttatctcgaagggggcagatgccaatctgtgcaaccataaaggaagacgaccactacacgttgtgaagcagtcgatcgacatggctcagatcatccaaagtctgattgatgcaggagcggatatcgatgcagcagagaaacatggactcactccgttgatgttcatgtgctccagagtgtctcttgaaggctcagcgacgttgctggccctgggagctgatgcccatcgtgttgcttggtcaaatggcttctcagctttggaatttgcagtaaagagcgggcgtacagagcttgtcgagctttgtctttcgaagggcgcaaatccgaacgcgcctacgttggacggaagcaccagtctgcacttagctgccgctctggctcacaccgatatcattctccggttgctgcagggtggtgcgaaccccaacatccagaacaggtgtgggcagactccagcgactattctgcttgcgtcagctcccaacggaagtggtgatgcccgaatcttgtgcctggagatcttgacgtgcgctgggtgtcatctcgatcaacgagatctctttggccgccaagcttctcacttagcgaacatctcgcgagattttcgcgtggtcgaattgctgcgaaagttgggatcgcttaatcggggaggcaatggcgttgatgtcgacatctttggctgctcttctgctgactacactgaccatatcgaggtaaacaaaacggcgggatttctgcgttcgctaatgcctaacgactcgtggaagattggagacaaggatgaccacgcttcacggtctgtacgctcgagttctattgacggaatgattcgcgacctggttactgggatggacgtcgatttggccgatgtggtcgcgtttgtgctgttcctcgacagcttttcaagcctaaacgaagtggtggatcgggtatacgaacatgtccgcaatggagtcaagagtcacggactcattcgtcttttcataatgatattgctgttcaggcagtcagaatcgaaggagagtgacgatctgcgtgatcgtttctatagcctcgttggccacgctgttgacgtttcgaaagagaagttggtgcctttggttgaagaattcacagctctttacaagggatatttttccctgcgtggggctggaaaatacgcactgaactacgaaatgatccctggaatgatgaccacattatacggcttagaatctggaatcgaacttgtttccaacgagcccttctcgaggcatttgcaccgatatatcgacgcagagcagtgggcagaacaatgcacacttctgacgcacgcagtcttctgcaagattccagtccacgatttgatctcgccaggctcgaaaaggcagcactcagttgagttcaccagcatcaaacgatggttccaacacatctctgcgtacgtcatcaacgctgttcttgttcaagatacccctgaagaacgggcagaagtgatttcgttttacctcaagctgagtgtggctgcgaagaaaaagatgaacgagatgcaactactctcagacaaaggatgtcgagagatgaaccgcctgatgcgtcagactacaaacccaagcatgccttacattggcctgtatcttcaaggcttcgtcggattgaacgagttgcctacgtttgacaagaacggtctggtaaatgcaagccggcttcgacgaatgggcgagctgtcgatggaaatcttgcatcgacagtctgtcgcctacactctccagaacgacgaggatgtcgacaaactgctgcacgtctcgttgccgtattcctccgaggagagtcggtatactcgttcgttggagctggaacctcgcgaggcggacgcgatgccgctgagcgaccgtggctcatgtgcattaatcgatgatgacctcgacctggaaagcgaagttcgtgagtcaatcggcggtgacgggactttcggtttccgtcagtggattcggaagcagcaagtagtgcaccggaatcgctctcggtcgtctctaattgctttgtatgagtgggtgtag

SEQ ID NO: 4 - protein, 1270 aa, Phytophthora ramourum
MASAWRQHPHDPWDLPSPPPLLEEAAPKASKRSHFSRRSTRAQSQARAQAQQAELEVEALADQRDRDERRSLLAAMFGGRSKSRTSRSSSDAPQTLVQREKAFRLVGNFVKMGWLTKQGHMWKSWKTRFFVLFSDGTFAYYKNKGRKKIKGCMQLNDGVVSVQHVDVRVADKAYVFQIEKGFYKLLCYCCSQFEAELWVAALRSVRRVTPPCYETDLTATEEKAGSNAVTRHLNKIFITDKQIAKSLVQFKVNEHDHSYAAIHNFIVELDDAIIDGHHLELYQDPEIELLPGNDLVRLIRRHVEDRVFIPLYAEAYASLETDKVKTRRSNLEQNLKVLRRKTQADFGISKDLSVCDWKLAINVINMLECVSLPTHKFEVILSAGKAITESIAQYNGDLFEVSDETLTAVFRYVVTMASLSDLPILRALLKYGYQHHPACQNKANVVSAFLDGIKWVECFETGDESYQFDSLALAGSRVSVSISTNDVGIQFTTDGNGRGAIVYSVRKQSQAALSASIVPGLSLIAINHEPVIGMPFDKIIQRVRTAALPKQLIFMTEFYYYQLLSLDSEMFRYLMCIAARRGDLDSAAWLRSSTVELNKLCSWEKSRGKQVFGFIPVSGKGSPLHAAVHNGQLSMVKYLISRGADTNLCNQKGRRPLHVVKQSIDMALIIESLIDAGADIDVAEKHGLTPLMFMCFRASLEGAATLVALGADVRRVAWSNGFSALEFAVKSERTELVELCLSKGANPNMPTLDGNTSLHMAAALPHADIILRLLQSGANPNVQNRYGQTPATVLLASAPGGGGDARILCLEILTCAGCRLDKRDLFGRQASHLANISRDSHVMGLLRKLGSLNRAGNSVDVDIFGCSPVDYSHGIEVSKATEYFGSQMPNNSWEIIDKTDHVSRSALSGSVEDLIRKLVAGTEVDLVDVVAFVLFLDSFSSLNELVDRLSEHVRNGIKSHGLIRLFIMILLFRNTETKEVDDLSDRFYSLISHNIDVPREKLALSSYPTSPFRFICTERWAEQCTLLTHAVFCQIPVQDLILTGSKKQHSVEFTSIKRWFQHISAYVINAVLVQNSPEERAEVISFYLKLSAEAKKMMNEMQLLSDKGCREMNRLMRKTVNPSMPYIGLYLQGFVGLNELPAFGKEGQVNASRLRKMGELAMEILHRQSVAYTLQNDESIDKLLHVSMPYSSEESRYARSLEVEPREADAIPLSDRGSCVVVDDDLDLENEVRESVGGDGTFGFRQWIRKQQVVHRNRSRSSLIALYEWV

SEQ ID NO: 5 - protein, 1014 aa, Phytophthora sojae
MASGWRQKPRLDPWDPPSPPPLLEEAAPKPKPKTSRFSRRSTRAKSHARQQAQHVELEVEEHAEQRDRDKRRSLLASMFGERAQSRASRSSSDASALQSQTQTLVQREKAFRLVRHGSDPLLNDAFYATQVQVGNFVKMGWLTKQGHMWKSWKTRFFVLFSDGTFAYYKNKGRKKIKGCMQLNDGVVSVQHVDIRLADKAYVFQIEKGFYKLLCYCCSQFEAELWVAALRSVRRVAPPCYEMDLTATEEKAGSNAVTRHLNKIFITDKQIAKRLAEFKANTHDHSYAAIHNFIVELDDAIIDRHHLEFYQDAEIELLPGNELVRLIRRHVEDRVFIPLYAEAYASLETDKVKTRRSNLEQHLKVLKQKTQADFGISKDLSVCNWKQAISVVNMLDCVSLPTHKFEVILSAGKAIMELIAQYNGELFEVSDEMLTAIFRYVVTMSSLSDLPTLRALLKYGYQHHPASQNKANVVTAFLNAIKWVECFEAGDESYQFDSLALAGSRVSVSISTNDVGIQFTTDGNGRGAIVYNIRKQSQAALSAAIVPGLSLIAINHEPVIALEFAVKIDYSHRIEVDKSASYLGSQMPNSSWKMVDKDDHVSRSARPGSVDDLIRELVAGTEVDLVDVVSFVLFLDSFSSLNEVVDRLSAHVRNGVKSHGLIRLFIVVLLFRQSETKEADDLRDRFYSLISLNIDAKDKLVPLVEEYMSLYKEYFSLRGAGNYAVNYQAIPGMMSKVYGMESGFEPVSSGPFSAHLHRFVDAERWAEQCTLLTHAVFCKIPVRDFIPTGSKKKHSVEFTSIKRWFQHISAFVINAVLVQNTPEERAEVISFYLKLSLDAKKMMNEMQLLSDKGCREMNRLMRKTANPSMPYIGLYLQGFVGLNELPAFDKEGLVNANRLRRMGELAMEILHRQSVAYTLQHDEDVNKLLHVSLPYSSEESRYARSLELEPREADAIPLSDRGSYTLIDDDVDLENEVRESIGGDGTFGFRQWIRKQQVVHRNRSRSSVIALYEWV

SEQ ID NO: 6 - protein, 1343 aa, Phytophthora infestans
MASGWRHKGTKAVLDPWGSPSPPPLLDETAPKISKRSRFSRRSTHAKSQAQHQAQNIELEVEEFAEQQDRDKRRSLLASMFRTRALSRSSRSISDATEAPQALVQRDKGFRLVRHGSDPLLNDSFYATQVQVGNFVKMGWLTKQGHMWKSWKTRFFVLFSDGTFAYYKNKGRKKIKGCMQLNDGVVSVQHVDIRVAGKAYVFQIEKGFYKLLCYCCSQFEAELWVAALRSVRRVSPPCYEMDLTATEEKAGSNAVTRLLNKIFITDKEVAKNWVQFKENDHDHSYAAIHNFIVELDDSIIDRHHLELYQDPEIELLPGNELVRLIRRHVEDRVFIPLYAEAYASLETDKVKSRRSNLEQNVKVLKQKAQADFGISKDLSVCNWKQAISVINMLDSVSLPTHKFEVILSAGKTVTEEIAQYNGELFEVTDETLTAVFRYVVTMSSLSDLAILRALLKYGYQHHPACQNKANVVTAFLDAIKWVENFESGDESYRFDTMALAGSRVSVSISTNDIGIQLTTDGNGRGAIVYSVRKQSQAALSAAIVPGLSLIAINREPVIGMPFEKIIQRVRTAALPKQLTFMTEFYYYQLLSLDLEMSRYLTCIAARRGDLDSAGWLRSSTVELNTLCSWEKTRGKQVFGFIPASGKGSPLHAAVHNGQLGMVNYLISKGADANLCNHKGRRPLHVVKQSIDMAQIIQSLIDAGADIDAAEKHGLTPLMFMCSRVSLEGSATLLALGADAHRVAWSNGFSALEFAVKSGRTELVELCLSKGANPNAPTLDGSTSLHLAAALAHTDIILRLLQGGANPNIQNRCGQTPATILLASAPNGSGDARILCLEILTCAGCHLDQRDLFGRQASHLANISRDFRVVELLRKLGSLNRGGNGVDVDIFGCSSADYTDHIEVNKTAGFLRSLMPNDSWKIGDKDDHASRSVRSSSIDGMIRDLVTGMDVDLADVVAFVLFLDSFSSLNEVVDRVYEHVRNGVKSHGLIRLFIMILLFRQSESKESDDLRDRFYSLVGHAVDVSKEKLVPLVEEFTALYKGYFSLRGAGKYALNYEMIPGMMTTLYGLESGIELVSNEPFSRHLHRYIDAEQWAEQCTLLTHAVFCKIPVHDLISPGSKRQHSVEFTSIKRWFQHISAYVINAVLVQDTPEERAEVISFYLKLSVAAKKKMNEMQLLSDKGCREMNRLMRQTTNPSMPYIGLYLQGFVGLNELPTFDKNGLVNASRLRRMGELSMEILHRQSVAYTLQNDEDVDKLLHVSLPYSSEESRYTRSLELEPREADAMPLSDRGSCALIDDDLDLESEVRESIGGDGTFGFRQWIRKQQVVHRNRSRSSLIALYEWV

SEQ ID NO: 7 - DNA, 5140 bp, Phytophthora ramourum
caaagtccaacgcaagagaaaaagtgtaggctggatattgtggtccgtaatatcatgtgcatctccgcatacaccgccacatccgaccccgtctccgtcccataataccctatgccaattaaaccggctggcgaccttgacgttgtccgaaaaatacaagcaggtgccaagtcccagtttagtttctgccgcctcgatcgatggcttcagcttggcgccagcatccgcacgatccgtgggacctgccgtcgccgccgccgctactagaagaagccgcgcccaaagcttccaagcgctcacacttttcacgccgcagcacgcgcgcccagagccaagcgagagcccaagcgcagcaggcggagctcgaggtggaggcgctcgccgaccagcgcgaccgcgacgagcgccgcagcctcctggccgccatgttcggtggtcgcagcaagtcacgcacgtcgcgatcgtcctcggacgccccgcagacgctcgtgcagcgagaaaaggccttccgcctcgtgcgccacggctcggacccgctgctgaacgacgcgttctacgccacgcaggtgcaggtaggcaacttcgtcaagatgggctggctcaccaagcagggccacatgtggaagagctggaagacgcgcttttttgtgctcttctcggacggaacgttcgcctactacaagaacaagggccgcaagaaaatcaagggctgcatgcagctcaatgacggcgtcgtgtccgtgcaacacgtggacgtccgcgtggcggacaaggcctacgtattccagatagagaaggggttttacaagctgctgtgttattgctgcagccagtttgaggccgagctgtgggtcgcagcgctgcgctcggtgcggagagtgaccccgccctgttacgagacggatctaacggccactgaggagaaggcgggctccaacgcggtcacgagacacttgaacaagattttcatcacagataagcagatcgccaagagtttggtgcagttcaaggtgaacgagcacgaccattcgtacgcagcaattcacaattttattgtcgagctggacgacgcgatcattgatggtcatcatttggagctctatcaggacccagagattgagctgctgcctggtaatgacctggtacggctgattcgtcgacatgtggaagatcgagtgtttattccgttgtatgcggaggcgtacgcgagcttggagacggacaaggtcaagacgaggcgcagcaacctcgagcagaacctcaaggtgctgaggcggaaaactcaggcagattttggcatctcgaaggatttatccgtttgcgactggaagctagccatcaatgtgattaacatgctcgaatgcgtgtcgctcccaacgcacaagttcgaggtcattttatcagctggaaaggccatcacggagtctattgctcaatataacggcgacctgttcgaggtatcagatgagacgctgacggcggtttttcggtacgtagttacaatggcgtcactgagcgatttaccgattctgcgagcgcttctcaagtacggataccagcaccacccagcgtgtcagaacaaagcaaacgtcgtttcagctttcctcgatgggatcaaatgggtggagtgcttcgagactggcgatgaaagctatcagtttgattccttagcgctagcagggtctcgcgtgtcggtatccatctcgactaacgatgtcggcattcagtttacgaccgatggcaatggtagaggggctattgtttacagtgtgcgaaagcaatcacaagcagcacttagcgcttcaatcgtccctggattgtcgctgattgccatcaaccacgaaccagttatcgggatgcctttcgacaagattatccagcgcgtgcgaactgcggcactccctaagcaactcattttcatgaccgagttttactactaccagcttctttcgctggattcggagatgtttcgatacctaatgtgtattgcagcgcgccgtggggacttggattcggctgcttggctgcggtcgtccacggtggagctcaataaactttgctcgtgggaaaaatctcgaggaaaacaggtctttggattcatccctgtatctgggaagggatccccacttcacgcggcagtgcataacggtcaactctctatggtgaagtatcttatctcaagaggggctgatacgaacttgtgcaatcagaaaggaagacgcccacttcatgtcgtgaagcagtcgattgatatggcgttgatcattgagagccttattgatgcgggagctgacattgacgtagcggaaaaacacggtcttaccccgctaatgttcatgtgctttagagcatcgcttgaaggtgccgcgacgcttgtagcacttggagcagatgttcgccgtgttgcatggtcgaatggattttcggctctggagtttgcagtgaagagtgaacgaacagagctggtagagttgtgcctttcaaagggggcaaatccaaatatgcctacgctggacggaaacacaagtttgcatatggcagccgctctcccccacgctgatataatcctccggctcctacagagcggtgcaaaccccaatgttcagaatcggtatggtcagactccggccacagtcctacttgcgtctgctcctggtggaggtggcgatgcccgaatcctgtgtctggagattttaacatgtgccggttgccggttggacaaacgtgatctctttggtcgccaggcttctcacctggcaaacatctcgcgagattctcatgtcatgggtctacttcgaaagttgggatcgttgaaccgagcaggcaatagcgttgatgtcgatatcttcggttgctcacctgttgattatagtcatgggatcgaggtaagcaaggcgacagaatattttggatcgcaaatgccgaacaattcgtgggaaatcattgacaagaccgaccacgtgtctcgatctgcactctcgggttctgtcgaggatttgattcgcaagttagttgcaggcacggaagtggacttggtggatgtggtggcgttcgtgctcttcttggacagcttctcgagtcttaacgaattggtggatcggttgagcgagcatgtccgaaacgggattaaaagtaagttttgtgcgtgcttgtttgttaatccttcgcgcggtcaatatgcacctacattagacactaacgtggcatttacccatttgtattgttcgtataggccacggacttatccgtctcttcatcatgatcctgctttttagaaatacggaaacgaaggaagtagatgatctgtctgatcgcttctacagcctgatcagtcacaacattgacgtccctagggaaaagttggtacctcttgttgatgagtatacctccctttatagggaatatttctcattgcgaggagctggaaactacgccacaacttacgaaaggattccagaaatgatgactaagatatacggcttggagtcaggcattgagttcgtatccgacgagccctttccgcttcatttgcaccggtaagtcgagtgatagctcattgtctcccttttgatcgaacatatctgacctatggatttcggtcctctttcgttgggtaatatctcaggtatattgatgcagaacggtgggctgaacagtgcacgcttctgacgcacgcagtcttttgccagattccaggtgggatgttatcgtctgcttgtttagttggagggttgactaaactttgctgtctctaaaatgcagtccaagatttgattttgacaggctcgaaaaagcagcactccgttgaatttacgagtatcaaacgatggtttcagcacgtacgttctgttgtcgctttatatttacgtgctaaagagattaactttcgggcgtttttgtccgtggtaatattgtgtagatctctgcgtacgtgatcaacgctgttcttgtacaaaactccccagaagagcgtgccgaagtaatttcgttttatctcaaggtaaaaggatatttcggctcgtttgacctctggtgggctgcatgctaacctttgatttcgaactatgatcaggccgccgattattgcgtgtcattacacaatcatgatacgctggcttcgatcttatacgcattgcaatctactgcggtacaacggcttagaaagtcgatcgattgtgtacgttattaccagctatgttcctctcgtgggcacgaagtcacgaattctgatttggtggtgcattacagctaagtgcggaagcgaagaagatgatgaacgaaatgcagctactttccgacaaaggatgtcgcgagatgaaccggttgatgcgcaaaactgtcaaccccagtatgccttatattgggttgtatctccaggtaaagacgcgcatttcaaagtgatgccttcggacgaaaatgtctgacattgtgcgtgttgatttagggcttcgtcggactgaacgagttgcctgcgtttggcaaagagggtcaagtcaatgcgagtcggctacgaaaaatgggcgagttggcaatggaaatcttgcaccgccagtctgttgcgtacactcttcaaaacgacgagagtattgatgtgagtagcaattttgcctgtcgtcgtaatcctcagctgtggatatcacgtctaacctgcgtatgtaactcttttgcagaaactgttgcacgtttcaatgccgtattcgtcggaggagagtcgatatgctcggtcattggaggtcgagccgcgtgaagcagatgcgattccattgagcgatcgcggttcgtgtgttgttgttgatgacgacctcgaccttgaaaatgaagttcgcgagtcagttggcggcgatggaacctttggattccgccagtggattaggaagcagcaagtcgtgcaccgcaatcgctcacgctcttcgctgatagctttgtacgagtgggtgtagctgtgatgtgctgtcttctgcgccgtgctttggtgaatacttttatctattcgttgtggaatgttttcgcccaacattaatacattagcatatttagttatcagcgatgtacttctcgaagtcttccatcgacatcatggcgtccggatccagcgatagttcagcggggtccttttatcagcaaatggcgctcattaagt

SEQ ID NO: 8 - DNA, 4708 bp, Phytophthora sojae
atggcttcaggctggcggcagaaaccgcgcctcgatccgtgggacccgccgtcgcccccgccgctgctggaggaggccgcgcccaagcccaagcccaaaaccagcaggttctcgcgccgcagcacccgcgccaagagccacgcgcgccagcaggcgcagcacgtggagctggaggtggaggagcacgccgagcagcgcgatcgcgacaagcgccgcagcctgctggcctccatgttcggagagcgcgcgcagtcgcgcgcctcgcgctcgtcctcagacgccagcgcgctgcagtcgcagacgcagacgctcgtgcagcgcgagaaggccttccgcctggtgcgccacggctccgacccgctgctgaacgacgcgttctacgccacgcaggtgcaggtgggcaacttcgtcaagatggggtggctcaccaagcagggccacatgtggaagagctggaagacgcgctttttcgtgctcttctcggacggcaccttcgcctactacaagaacaagggccgcaagaagatcaagggctgcatgcagctcaacgatggcgtcgtgtccgtgcagcacgtggacatccgcctggccgacaaggcctacgtgttccagatcgagaagggcttctacaagctgctgtgctactgctgcagccagttcgaggcggagctgtgggtcgctgcactgaggtcggtgcgcagagtggcaccgccgtgctatgagatggacctgacggccactgaggagaaggccgggtccaatgcggtcaccaggcacttgaacaagatcttcatcacggacaagcagatcgccaagaggctggcggagttcaaggccaacacacatgaccactcgtacgcggccattcacaatttcatagtggagctggacgacgcgatcattgatcgtcaccatttggagttttatcaggacgcggagatcgagctcttgccgggcaacgagctggtgcggctgattcggcggcatgtggaagaccgcgtgttcattccgctgtatgcagaggcgtatgcgagcttggaaacggacaaggtgaaaacgaggcgcagcaacctcgagcagcatctcaaggtactgaagcagaaaacgcaggccgatttcggcatctcgaaggacctttcagtttgcaattggaagcaggctatcagcgttgttaacatgctggattgcgtgtcactacctacgcacaagtttgaagtcatcctatcagctggaaaagcgatcatggagctgatcgctcaatacaatggcgaacttttcgaagtgtcagacgagatgttaacggcgatcttccggtacgtggtaacgatgtcttcgctgagcgatctaccaactcttcgagcgctgctgaagtacggctatcagcatcatccagcatcgcaaaacaaagcgaatgtggttacggcctttctcaacgccatcaagtgggtggagtgtttcgaggccggcgacgagagttaccagttcgattcgttggcgctggctggatctcgagtttctgtatccatctcaaccaatgatgtcggcattcaattcactacggatggtaacgggcgaggagcaatcgtgtacaatatccgaaagcagtctcaggcagcgctgagcgctgcgattgtgcctggtctgtcattgatcgcgatcaaccacgagcccgtcattggtatgcctttcgacaagattattcaacgagtgcgcaccgcagcgctcccaaagcaactcactttcatgacggagttttattactaccaactactctcgctagactcggagatgtatcagtacctgatgtgtattgcagctcgacgtggtgacttggactctgctgcctggatgcgatcctcaacgattgaactcaatacgctatgctcgtgggagaagtctcgtggaaaacaggtttttgggttcacacctgtgtctggaaaaggatcgccacttcatgcagcagtacataacggacagcttagcatggtgaactaccttatttcaagaggagcagacgttaatctgtgcaaccagaagggacggcgcccactgcacgttgtgaagcagtcaatcgacatggccatgattattcaaagcttgatcgatgccggggctgatatcgacgcgatggagaaacacgggcttaccccgttaatgttcatgtgctccagagcatctctcgaaggatcagcaacactcctagcactcggtgcagatgttcactgtgttgcgtggacaaacggattttcagcactggagtttgcggtgaaaagtgagcataccgaattggtcgagctgtgtctctccaagggggcgaatccgaatgccccaacgttggatgggaacaccagtctacatttggccgccactcaggccaacactgatatcattcttcgacttttgcaaggtggagcaaaccccaacgttcaaaatcggtacggacaaacgccggcagcactattgcttgcgtcttcacccggcggaagtagcgacgtacgcatcctatgcctcgaaattttgacgtgtgctggctgccgccttgacaaaagagatctatttggccgccaggcttctcatctcgccagtatttcaagagactctcacgttgtcgatctacttcgcaagttgggctcgctgaatcgggaagccaatggtgttgaaattgacatcttcggttgttcttcagtcgactatagtcaccgtattgaggtggacaagtctgcaagctatctggggtctcaaatgcccaacagttcgtggaaaatggtggacaaggacgaccatgtgtctcgatctgcacgcccgggctcggttgatgatctgatccgcgagttagtggcagggacggaagtggacttggtggatgtggtgtctttcgtgctatttctcgatagcttttcgagcttgaacgaagtagtggatcgcctgagcgcacacgtccgaaacggtgttaaaagtacgttcatgcttttggatcgaatgatatcgttaacggtatcggtgtgaaggtctaaactaatggttgctgtttgttttgtttaggccacggacttatccgtctctttattgtggttctgctcttcaggcaatcggaaaccaaggaagctgacgatctgcgtgatcgtttctacagcctcattagtcttaacattgacgcgaaggacaaactggtccctctggtcgaagagtacatgtcactttacaaggagtatttctccttgcgaggagctggaaattacgcggtgaactaccaagcgattccaggaatgatgagcaaggtttacggaatggagtcaggcttcgagcccgtatcctctggacccttttcggctcatttgcatcggtaagttactcgcgaagtatatatgacccactgactctgacatatttacatgtctgtcggatggcaggttcgtcgacgcagaacggtgggcggagcagtgcaccctcttaacgcacgcagtgttctgcaagattccgggtaagtcctttattacgctgatgcgtgtgaataaaaaaactaatggcgcatttttttgtagtccgagacttcattccaactggatcgaagaaaaagcactcggttgagtttacgagcattaaacgctggttccagcacgtacgttcccttagtaaccctactcaggctgctgaagtgataactaatctactgtgttaccatgtagatctctgcatttgtgatcaatgctgtgctggttcagaataccccggaagaacgtgccgaagtgatttcgttttatctcaaggtactcacaatgccaaaggcgatgaaagtttctggcacaaggtttaatttttgccttattgtgaaatgtgctcaggtcgccgattactgtatttcattgcacaaccacgacactctcgcttccattttatacgcattgcaatctacagcagtccagcgacttcgcaagacaatcgattgtgtgagttaacggtttgtgttggtctgctttgacattgaagctgttgctgacccgtgtgaggtgttatattgtaaatttcagctaagtctggacgcgaagaaaatgatgaacgagatgcagctactttcagacaagggttgccgagagatgaatagactgatgcgcaaaaccgctaatccgagcatgccatacatcggactgtacctccaggtaggtgttatctgcgtgaactattcgattgcgaggcgagtgactgacttgtccattatattattttagggctttgtcggactcaacgagttgcctgcttttgacaaagagggcctggtgaacgcgaatcgtctacgaaggatgggcgagctggccatggaaatcctgcaccgccagtctgttgcctacacgcttcagcacgacgaagatgttaacgtgagttgcagtatatcacggctgctctcaactttatagctaacttgtacctgtatgccatgtagaaactgctgcacgtttccctgccgtactcttctgaagagagtcggtatgctcggtcattggagttagagccgcgtgaagctgacgcgatcccattgagcgatcgtggttcgtatactttgatcgatgacgatgttgacctcgaaaatgaagttcgtgagtcaatcggcggtgacgggactttcggcttccgtcagtggatccggaagcagcaggtagtgcatcgaaaccgttcgcggtcgtcggtgatagctctgtacgagtgggtgtag

SEQ ID NO: 9 - DNA, 4708 bp, Phytophthora infestans
gttacccaagcgatggcttccggctggcgccacaaaggcaccaaagccgtcctggatccgtgggggtctccatcgcctcctccactactcgacgagacggctcctaagatcagcaagcgctcccgcttctcacgtcgcagcacacacgccaaaagccaagcgcaacaccaagctcagaacatcgagctcgaagttgaagaatttgccgaacaacaggaccgtgacaaacgtcgcagcctcttagcctccatgttcagaacccgagcactgtcgcgctcatcgcgctccatatcggacgccacggaggcgccacaggcgctcgtccagcgagataaaggtttccgactggtgcgtcacggctccgatccactcctaaacgactcgttttacgccactcaagtgcaggttggcaatttcgtcaagatgggctggctcaccaaacaaggacacatgtggaagagctggaagacgcgcttcttcgtgcttttctcggatggcacttttgcctactacaagaacaagggcaggaagaagatcaaaggttgcatgcaactaaacgacggcgtggtttccgtgcagcacgtggacatccgagtagctggtaaagcgtatgtgttccagatcgagaaaggtttctacaaattattgtgctattgctgcagccagtttgaagccgagttgtgggtcgctgcattgcggtcagtgaggagggtatctccaccctgttatgaaatggacctgactgcgaccgaagagaaagcaggatccaatgcagttaccagactcttgaacaagatcttcatcacggacaaagaggtggctaaaaactgggtccagttcaaggaaaatgatcacgaccattcgtacgctgcaatccacaatttcatcgtggaattggacgactctatcatcgatcgtcaccatttggagctctatcaagatccagagattgaattattgccaggtaatgagctcgtgcgactgattcggcgacatgtcgaggaccgtgtgtttattccgttgtatgcggaggcatacgcgagcttagagacggataaagtcaagtcgagacggagcaacttggagcagaacgttaaggtgttgaaacagaaagcacaggcggatttcggcatctcgaaagacttgtcggtttgcaattggaagcaagctatcagtgtgatcaatatgctcgactctgtgtcgctgccgacgcacaaattcgaggtcattttgtccgccgggaagaccgtcacagaggagattgctcagtataatggcgagctcttcgaggtcacagacgagacgttgacggcggtgttccggtacgtggtaacgatgtcctcgctcagcgacttagcgattcttcgagcactactgaagtatggatatcagcaccacccagcatgtcagaataaagcgaatgtcgtgactgcttttctcgatgcgatcaaatgggttgagaactttgagtctggagatgaaagctaccgattcgacacgatggcgctggcaggatctcgtgtctccgtatccatttcgaccaacgatattgggattcagctcacgacagacggaaatggaagaggagctattgtttatagtgttcggaagcagtctcaagcagctttgagcgctgccatcgtgcctggattgtcgctgatcgcgattaaccgtgagcctgtgattggaatgcctttcgagaaaattatccaacgcgttcggactgcagcattgccgaagcaacttacgttcatgacggagttttactattatcagctgttgtcactggacttggagatgtcccgctatttgacgtgcattgcagctcgtcgtggagacttggactcggctggatggctgcgatcctcgactgtggaattgaacactctttgctcgtgggaaaagactcgaggcaagcaggttttcggattcattccagcatccggcaagggctcgccgcttcatgctgctgtgcacaatggacaactcggaatggttaattatcttatctcgaagggggcagatgccaatctgtgcaaccataaaggaagacgaccactacacgttgtgaagcagtcgatcgacatggctcagatcatccaaagtctgattgatgcaggagcggatatcgatgcagcagagaaacatggactcactccgttgatgttcatgtgctccagagtgtctcttgaaggctcagcgacgttgctggccctgggagctgatgcccatcgtgttgcttggtcaaatggcttctcagctttggaatttgcagtaaagagcgggcgtacagagcttgtcgagctttgtctttcgaagggcgcaaatccgaacgcgcctacgttggacggaagcaccagtctgcacttagctgccgctctggctcacaccgatatcattctccggttgctgcagggtggtgcgaaccccaacatccagaacaggtgtgggcagactccagcgactattctgcttgcgtcagctcccaacggaagtggtgatgcccgaatcttgtgcctggagatcttgacgtgcgctgggtgtcatctcgatcaacgagatctctttggccgccaagcttctcacttagcgaacatctcgcgagattttcgcgtggtcgaattgctgcgaaagttgggatcgcttaatcggggaggcaatggcgttgatgtcgacatctttggctgctcttctgctgactacactgaccatatcgaggtaaacaaaacggcgggatttctgcgttcgctaatgcctaacgactcgtggaagattggagacaaggatgaccacgcttcacggtctgtacgctcgagttctattgacggaatgattcgcgacctggttactgggatggacgtcgatttggccgatgtggtcgcgtttgtgctgttcctcgacagcttttcaagcctaaacgaagtggtggatcgggtatacgaacatgtccgcaatggagtcaagagtaagtgcgcgcttttgatttactaatggacacaacgctgaccttgttgttatcgtgtttttctaggtcacggactcattcgtcttttcataatgatattgctgttcaggcagtcagaatcgaaggagagtgacgatctgcgtgatcgtttctatagcctcgttggccacgctgttgacgtttcgaaagagaagttggtgcctttggttgaagaattcacagctctttacaagggatatttttccctgcgtggggctggaaaatacgcactgaactacgaaatgatccctggaatgatgaccacattatacggcttagaatctggaatcgaacttgtttccaacgagcccttctcgaggcatttgcaccggtaagtcgaagatttgaggcttgtatggcttgagcgttactgattttggtttgcttttcggcagatatatcgacgcagagcagtgggcagaacaatgcacacttctgacgcacgcagtcttctgcaagattccaggtgagcttccatttggacacagctgttgtgaagatggagctgatcttttgttgtgtatgatcatctgtactgtagtccacgatttgatctcgccaggctcgaaaaggcagcactcagttgagttcaccagcatcaaacgatggttccaacacgtacgtgatgctaaatatttacatagtctgggggttattactgaagtgcttgtacctgtcgtatttttgtagatctctgcgtacgtcatcaacgctgttcttgttcaagatacccctgaagaacgggcagaagtgatttcgttttacctcaaggtgcgatgactttaccgtactacttcgaagtatattaaagaggtactcggtgtgctaaactgtacgtatactatgatcaggccgcggaatactgcatttccttacacaaccatgacacgctggcttcgattttatacgctctgcaatctactgcggttcagcgtcttcgaaagacgatcgactgtgtacgattccataaaagcatgtacctctgtgatggctggttgtataactcgtgttttacttgcgtattgtagctgagtgtggctgcgaagaaaaagatgaacgagatgcaactactctcagacaaaggatgtcgagagatgaaccgcctgatgcgtcagactacaaacccaagcatgccttacattggcctgtatcttcaagtgagtgtattctcatcgttacgttatggtaaacactacatgtactaatctcgtatatatcttcaagggcttcgtcggattgaacgagttgcctacgtttgacaagaacggtctggtaaatgcaagccggcttcgacgaatgggcgagctgtcgatggaaatcttgcatcgacagtctgtcgcctacactctccagaacgacgaggatgtcgacgtaagttgcgatgcgatctcgggtcttggtgtcactttctaattatctcttttgttgctgtagaaactgctgcacgtctcgttgccgtattcctccgaggagagtcggtatactcgttcgttggagctggaacctcgcgaggcggacgcgatgccgctgagcgaccgtggctcatgtgcattaatcgatgatgacctcgacctggaaagcgaagttcgtgagtcaatcggcggtgacgggactttcggtttccgtcagtggattcggaagcagcaagtagtgcaccggaatcgctctcggtcgtctctaattgctttgtatgagtgggtgtag

SEQ ID NO: 10 - DNA, 3109 bp, Artificial Sequence
  Derived from Phytophthora sojae
ggatcctctagaatggcaagcggttggcgtcagaaaccgcgtctggacccatgggaccctccaagcccgccgccgctgctggaagaagcagcaccgaaaccgaaaccgaaaaccagccgttttagccgtcgtagcacccgtgcgaaaagccatgcgcgtcagcaggcgcagcatgtggaactggaagtggaagaacatgcggaacagcgtgatcgtgataaacgtcgtagcctgctggcgagcatgtttggtgaacgtgcacagagccgtgcaagccgtagcagcagcgatgcaagcgcactgcagagccagacccagaccctggtgcagcgtgaaaaagcgtttcgtctggtgcgtcatggcagcgatccgctgctgaacgatgcgttttatgcgacccaggtgcaagtgggcaactttgtgaaaatgggctggctgaccaaacagggccatatgtggaaaagctggaaaacccgtttctttgtgctgtttagcgatggcacctttgcgtattataaaaacaaaggccgtaagaaaatcaaaggctgcatgcagctgaacgatggcgttgtgagcgtgcagcatgtggatattcgtctggcggataaagcgtatgtgtttcagattgaaaaaggcttttataaactgctgtgctattgctgcagccagtttgaagcggaactgtgggtggcggcgctgcgtagcgtgcgtcgtgtggcgccgccgtgctatgaaatggatctgaccgcgaccgaagaaaaagcgggcagcaacgcggtgacccgtcatctgaacaaaatttttattaccgataaacagattgcgaaacgtctggcggaatttaaagcgaacacccatgatcatagctatgcggcgattcataactttattgtggaactggatgatgcgattattgatcgtcatcatctggaattttatcaggatgcggaaattgaactgctgccgggcaacgaactggtgcgtctgattcgtcgtcatgtggaagatcgtgtgtttattccgctgtatgcggaagcgtatgcgagcctggaaaccgataaagtgaaaacccgtcgtagcaacctggaacagcatctgaaagtgctgaaacagaaaacccaggcggattttggcattagcaaagatctgagcgtgtgcaactggaaacaggcgattagcgtggtgaacatgctggattgcgtgagcctgccgacccataaatttgaagtgattctgagcgcgggcaaagcgattatggaactgattgcgcagtataacggcgaactgtttgaagtgagcgatgaaatgctgaccgcgatttttcgttatgtggtgaccatgagcagcctgagcgatctgccgaccctgcgtgcgctgctgaaatatggctatcagcatcatccggcgagccagaacaaagcgaacgtggtgaccgcgtttctgaacgcgattaaatgggtggaatgctttgaagcgggcgatgaaagctatcagtttgatagcctggcgctggcgggcagccgtgtgagcgtgagcattagcaccaacgatgtgggcattcagtttaccaccgatggcaacggccgtggcgcgattgtgtataacattcgtaaacagagccaggcggcgctgagcgcggcgattgtgccgggcctgagcctgattgcgattaaccatgaaccggtgattgcgctggaatttgcggtgaaaattgattatagccatcgtattgaagtggataaaagcgcgagctatctgggcagccagatgccgaacagcagctggaaaatggtggataaagatgatcatgtgagccgtagcgcgcgtccgggcagcgtggatgatctgattcgtgaactggtggcgggcaccgaagtggatctggtggatgttgtgagctttgtgctgtttctggatagctttagcagcctgaacgaagtggtggatcgtctgagcgcgcatgtgcgtaacggcgtgaaaagccatggcctgattcgtctgtttattgtggtgctgctgtttcgtcagagcgaaaccaaagaagcggatgatctgcgtgatcgtttttatagcctgattagcctgaacattgatgcgaaagataaactggtgccactggtggaagaatatatgagcctgtataaagaatattttagcctgcgtggcgcgggcaactatgcggtgaactatcaggcgattccgggcatgatgagcaaagtgtatggcatggaaagcggctttgaacctgtgagcagcggcccgtttagcgcgcatctgcatcgttttgtggatgcggaacgttgggcggaacagtgcaccctgctgacccatgcggtgttttgcaaaattccggtgcgtgattttattccgaccggtagcaagaaaaagcatagcgtggaatttaccagcattaaacgttggtttcagcatattagcgcgtttgtgattaacgcggtgctggtgcagaacaccccggaagaacgtgcggaagtgattagcttttatctgaaactgagcctggatgcaaagaaaatgatgaacgaaatgcagctgctgagcgataaaggctgccgtgaaatgaaccgtctgatgcgtaaaaccgcgaacccgagcatgccgtatattggcctgtatctgcagggctttgtgggcctgaacgaactgccggcgtttgataaagaaggcctggtgaacgcgaaccgtctgcgtcgtatgggcgaactggcgatggaaattctgcatcgtcagagcgtggcgtataccctgcagcatgatgaagatgtgaacaaactgctgcatgtgagcctgccgtatagcagcgaagaaagccgttatgcgcgtagcctggaactggaaccgcgtgaagcggatgcgattccgctgagcgatcgtggcagctataccctgattgatgatgatgtggatctggaaaacgaagtgcgtgaaagcattggcggcgatggcacctttggctttcgtcagtggattcgtaaacagcaggtggtgcatcgtaaccgtagccgtagcagcgtgattgcgctgtatgaatgggtgggcggcggcgaattccatcatcatcatcatcatcatcatcatcatcattaagctt

SEQ ID NO: 11 - DNA, 1022 bp, Phytophthora sojae
agcgcacacgtccgaaacggtgttaaaagccacggacttatccgtctctttattgtggttctgctcttcaggcaatcggaaaccaaggaagctgacgatctgcgtgatcgtttctacagcctcattagtcttaacattgacgcgaaggacaaactggtccctctggtcgaagagtacatgtcactttacaaggagtatttctccttgcgaggagctggaaattacgcggtgaactaccaagcgattccaggaatgatgagcaaggtttacggaatggagtcaggcttcgagcccgtatcctctggacccttttcggctcatttgcatcggttcgtcgacgcagaacggtgggcggagcagtgcaccctcttaacgcacgcagtgttctgcaagattccggtccgagacttcattccaactggatcgaagaaaaagcactcggttgagtttacgagcattaaacgctggttccagcacatctctgcatttgtgatcaatgctgtgctggttcagaataccccggaagaacgtgccgaagtgatttcgttttatctcaaggtcgccgattactgtatttcattgcacaaccacgacactctcgcttccattttatacgcattgcaatctacagcagtccagcgacttcgcaagacaatcgattgtctaagtctggacgcgaagaaaatgatgaacgagatgcagctactttcagacaagggttgccgagagatgaatagactgatgcgcaaaaccgctaatccgagcatgccatacatcggactgtacctccagggctttgtcggactcaacgagttgcctgcttttgacaaagagggcctggtgaacgcgaatcgtctacgaaggatgggcgagctggccatggaaatcctgcaccgccagtctgttgcctacacgcttcagcacgacgaagatgttaacaaactgctgcacgtttccctgccgtactcttctgaagagagtcggtatgctcggtcattggagttagagccgcgtgaagctga

SEQ ID NO: 12 - protein, 35 aa, Phytophthora sojae
VADYCISLHNHDTLASILYALQSTAVQRLRKTIDC

SEQ ID NO: 13 - DNA, 3945 bp, Phytophthora sojae
atggcttcaggctggcggcagaaaccgcgcctcgatccgtgggacccgccgtcgcccccgccgctgctggaggaggccgcgcccaagcccaagcccaaaaccagcaggttctcgcgccgcagcacccgcgccaagagccacgcgcgccagcaggcgcagcacgtggagctggagcgcgagaaggccttccgcctggtgcgccacggctccgacccgctgctgaacgacgcgttctacgccacgcaggtgcaggtgggcaacttcgtcaagatggggtggctcaccaagcagggccacatgtggaagagctggaagacgcgctttttcgtgctcttctcggacggcaccttcgcctactacaagaacaagggccgcaagaagatcaagggctgcatgcagctcaacgatggcgtcgtgtccgtgcagcacgtggacatccgcctggccgacaaggcctacgtgttccagatcgagaagggcttctacaagctgctgtgctactgctgcagccagttcgaggcggagctgtgggtcgctgcactgaggtcggtgcgcagagtggcaccgccgtgctatgagatggacctgacggccactgaggagaaggccgggtccaatgcggtcaccaggcacttgaacaagatcttcatcacggacaagcagatcgccaagaggctggcggagttcaaggccaacacacatgaccactcgtacgcggccattcacaatttcatagtggagctggacgacgcgatcattgatcgtcaccatttggagttttatcaggacgcggagatcgagctcttgccgggcaacgagctggtgcggctgattcggcggcatgtggaagaccgcgtgttcattccgctgtatgcagaggcgtatgcgagcttggaaacggacaaggtgaaaacgaggcgcagcaacctcgagcagcatctcaaggtactgaagcagaaaacgcaggccgatttcggcatctcgaaggacctttcagtttgcaattggaagcaggctatcagcgttgttaacatgctggattgcgtgtcactacctacgcacaagtttgaagtcatcctatcagctggaaaagcgatcatggagctgatcgctcaatacaatggcgaacttttcgaagtgtcagacgagatgttaacggcgatcttccggtacgtggtaacgatgtcttcgctgagcgatctaccaactcttcgagcgctgctgaagtacggctatcagcatcatccagcatcgcaaaacaaagcgaatgtggttacggcctttctcaacgccatcaagtgggtggagtgtttcgaggccggcgacgagagttaccagttcgattcgttggcgctggctggatctcgagtttctgtatccatctcaaccaatgatgtcggcattcaattcactacggatggtaacgggcgaggagcaatcgtgtacaatatccgaaagcagtctcaggcagcgctgagcgctgcgattgtgcctggtctgtcattgatcgcgatcaaccacgagcccgtcattggtatgcctttcgacaagattattcaacgagtgcgcaccgcagcgctcccaaagcaactcactttcatgacggagttttattactaccaactactctcgctagactcggagatgtatcagtacctgatgtgtattgcagctcgacgtggtgacttggactctgctgcctggatgcgatcctcaacgattgaactcaatacgctatgctcgtgggagaagtctcgtggaaaacaggtttttgggttcacacctgtgtctggaaaaggatcgccacttcatgcagcagtacataacggacagcttagcatggtgaactaccttatttcaagaggagcagacgttaatctgtgcaaccagaagggacggcgcccactgcacgttgtgaagcagtcaatcgacatggccatgattattcaaagcttgatcgatgccggggctgatatcgacgcgatggagaaacacgggcttaccccgttaatgttcatgtgctccagagcatctctcgaaggatcagcaacactcctagcactcggtgcagatgttcactgtgttgcgtggacaaacggattttcagcactggagtttgcggtgaaaagtgagcataccgaattggtcgagctgtgtctctccaagggggcgaatccgaatgccccaacgttggatgggaacaccagtctacatttggccgccactcaggccaacactgatatcattcttcgacttttgcaaggtggagcaaaccccaacgttcaaaatcggtacggacaaacgccggcagcactattgcttgcgtcttcacccggcggaagtagcgacgtacgcatcctatgcctcgaaattttgacgtgtgctggctgccgccttgacaaaagagatctatttggccgccaggcttctcatctcgccagtatttcaagagactctcacgttgtcgatctacttcgcaagttgggctcgctgaatcgggaagccaatggtgttgaaattgacatcttcggttgttcttcagtcgactatagtcaccgtattgaggtggacaagtctgcaagctatctggggtctcaaatgcccaacagttcgtggaaaatggtggacaaggacgaccatgtgtctcgatctgcacgcccgggctcggttgatgatctgatccgcgagttagtggcagggacggaagtggacttggtggatgtggtgtctttcgtgctatttctcgatagcttttcgagcttgaacgaagtagtggatcgcctgagcgcacacgtccgaaacggtgttaaaagccacggacttatccgtctctttattgtggttctgctcttcaggcaatcggaaaccaaggaagctgacgatctgcgtgatcgtttctacagcctcattagtcttaacattgacgcgaaggacaaactggagtatttctccttgcgaggagctggaaattacgcggtgaactaccaagcgattccaggaatgatgagcaaggtttacggaatggagtcaggcttcgagcccgtatcctctggacccttttcggctcatttgcatcggttcgtcgacgcagaacggtgggcggagcagtgcaccctcttaacgcacgcagtgttctgcaagattccggtccgagacttcattccaactggatcgaagaaaaagcactcggttgagtttacgagcattaaacgctggttccagcacatctctgcatttgtgatcaatgctgtgctggttcagaataccccggaagaacgtgccgaagtgatttcgttttatctcaaggtcgccgattactgtatttcattgcacaaccacgacactctcgcttccattttatacgcattgcaatctacagcagtccagcgacttcgcaagacaatcgattgtctaagtctggacgcgaagaaaatgatgaacgagatgcagctactttcagacaagggttgccgagagatgaatagactgatgcgcaaaaccgctaatccgagcatgccatacatcggactgtacctccagggctttgtcggactcaacgagttgcctgcttttgacaaagagggcctggtgaacgcgaatcgtctacgaaggatgggcgagctggccatggaaatcctgcaccgccagtctgttgcctacacgcttcagcacgacgaagatgttaacaaactgctgcacgtttccctgccgtactcttctgaagagagtcggtatgctcggtcattggagttagagccgcgtgaagctgacgcgatcccattgagcgatcgtggttcgtatactttgatcgatgacgatgttgacctcgaaaatgaagttcgtgagtcaatcggcggtgacgggactttcggcttccgtcagtggatccggaagcagcaggtagtgcatcgaaaccgttcgcggtcgtcggtgatagctctgtac

SEQ ID NO: 14 - protein, 1315 aa, Phytophthora sojae
MASGWRQKPRLDPWDPPSPPPLLEEAAPKPKPKTSRFSRRSTRAKSHARQQAQHVELEREKAFRLVRHGSDPLLNDAFYATQVQVGNFVKMGWLTKQGHMWKSWKTRFFVLFSDGTFAYYKNKGRKKIKGCMQLNDGVVSVQHVDIRLADKAYVFQIEKGFYKLLCYCCSQFEAELWVAALRSVRRVAPPCYEMDLTATEEKAGSNAVTRHLNKIFITDKQIAKRLAEFKANTHDHSYAAIHNFIVELDDAIIDRHHLEFYQDAEIELLPGNELVRLIRRHVEDRVFIPLYAEAYASLETDKVKTRRSNLEQHLKVLKQKTQADFGISKDLSVCNWKQAISVVNMLDCVSLPTHKFEVILSAGKAIMELIAQYNGELFEVSDEMLTAIFRYVVTMSSLSDLPTLRALLKYGYQHHPASQNKANVVTAFLNAIKWVECFEAGDESYQFDSLALAGSRVSVSISTNDVGIQFTTDGNGRGAIVYNIRKQSQAALSAAIVPGLSLIAINHEPVIGMPFDKIIQRVRTAALPKQLTFMTEFYYYQLLSLDSEMYQYLMCIAARRGDLDSAAWMRSSTIELNTLCSWEKSRGKQVFGFTPVSGKGSPLHAAVHNGQLSMVNYLISRGADVNLCNQKGRRPLHVVKQSIDMAMIIQSLIDAGADIDAMEKHGLTPLMFMCSRASLEGSATLLALGADVHCVAWTNGFSALEFAVKSEHTELVELCLSKGANPNAPTLDGNTSLHLAATQANTDIILRLLQGGANPNVQNRYGQTPAALLLASSPGGSSDVRILCLEILTCAGCRLDKRDLFGRQASHLASISRDSHVVDLLRKLGSLNREANGVEIDIFGCSSVDYSHRIEVDKSASYLGSQMPNSSWKMVDKDDHVSRSARPGSVDDLIRELVAGTEVDLVDVVSFVLFLDSFSSLNEVVDRLSAHVRNGVKSHGLIRLFIVVLLFRQSETKEADDLRDRFYSLISLNIDAKDKLEYFSLRGAGNYAVNYQAIPGMMSKVYGMESGFEPVSSGPFSAHLHRFVDAERWAEQCTLLTHAVFCKIPVRDFIPTGSKKKHSVEFTSIKRWFQHISAFVINAVLVQNTPEERAEVISFYLKVADYCISLHNHDTLASILYALQSTAVQRLRKTIDCLSLDAKKMMNEMQLLSDKGCREMNRLMRKTANPSMPYIGLYLQGFVGLNELPAFDKEGLVNANRLRRMGELAMEILHRQSVAYTLQHDEDVNKLLHVSLPYSSEESRYARSLELEPREADAIPLSDRGSYTLIDDDVDLENEVRESIGGDGTFGFRQWIRKQQVVHRNRSRSSVIALY

SEQ ID NO: 15 - DNA, 3214 bp, Artificial Sequence
  Derived from Phytophthora sojae
ggatcctctagaatggcaagcggttggcgtcagaaaccgcgtctggacccatgggaccctccaagcccgccgccgctgctggaagaagcagcaccgaaaccgaaaccgaaaaccagccgttttagccgtcgtagcacccgtgcgaaaagccatgcgcgtcagcaggcgcagcatgtggaactggaagtggaagaacatgcggaacagcgtgatcgtgataaacgtcgtagcctgctggcgagcatgtttggtgaacgtgcacagagccgtgcaagccgtagcagcagcgatgcaagcgcactgcagagccagacccagaccctggtgcagcgtgaaaaagcgtttcgtctggtgcgtcatggcagcgatccgctgctgaacgatgcgttttatgcgacccaggtgcaagtgggcaactttgtgaaaatgggctggctgaccaaacagggccatatgtggaaaagctggaaaacccgtttctttgtgctgtttagcgatggcacctttgcgtattataaaaacaaaggccgtaagaaaatcaaaggctgcatgcagctgaacgatggcgttgtgagcgtgcagcatgtggatattcgtctggcggataaagcgtatgtgtttcagattgaaaaaggcttttataaactgctgtgctattgctgcagccagtttgaagcggaactgtgggtggcggcgctgcgtagcgtgcgtcgtgtggcgccgccgtgctatgaaatggatctgaccgcgaccgaagaaaaagcgggcagcaacgcggtgacccgtcatctgaacaaaatttttattaccgataaacagattgcgaaacgtctggcggaatttaaagcgaacacccatgatcatagctatgcggcgattcataactttattgtggaactggatgatgcgattattgatcgtcatcatctggaattttatcaggatgcggaaattgaactgctgccgggcaacgaactggtgcgtctgattcgtcgtcatgtggaagatcgtgtgtttattccgctgtatgcggaagcgtatgcgagcctggaaaccgataaagtgaaaacccgtcgtagcaacctggaacagcatctgaaagtgctgaaacagaaaacccaggcggattttggcattagcaaagatctgagcgtgtgcaactggaaacaggcgattagcgtggtgaacatgctggattgcgtgagcctgccgacccataaatttgaagtgattctgagcgcgggcaaagcgattatggaactgattgcgcagtataacggcgaactgtttgaagtgagcgatgaaatgctgaccgcgatttttcgttatgtggtgaccatgagcagcctgagcgatctgccgaccctgcgtgcgctgctgaaatatggctatcagcatcatccggcgagccagaacaaagcgaacgtggtgaccgcgtttctgaacgcgattaaatgggtggaatgctttgaagcgggcgatgaaagctatcagtttgatagcctggcgctggcgggcagccgtgtgagcgtgagcattagcaccaacgatgtgggcattcagtttaccaccgatggcaacggccgtggcgcgattgtgtataacattcgtaaacagagccaggcggcgctgagcgcggcgattgtgccgggcgtggcggattattgcattagcctgcataaccatgataccctggcgagcattctgtatgcgctgcagagcaccgcggtgcagcgcctgcgcaaaaccattgattgcctgagcctgattgcgattaaccatgaaccggtgattgcgctggaatttgcggtgaaaattgattatagccatcgtattgaagtggataaaagcgcgagctatctgggcagccagatgccgaacagcagctggaaaatggtggataaagatgatcatgtgagccgtagcgcgcgtccgggcagcgtggatgatctgattcgtgaactggtggcgggcaccgaagtggatctggtggatgttgtgagctttgtgctgtttctggatagctttagcagcctgaacgaagtggtggatcgtctgagcgcgcatgtgcgtaacggcgtgaaaagccatggcctgattcgtctgtttattgtggtgctgctgtttcgtcagagcgaaaccaaagaagcggatgatctgcgtgatcgtttttatagcctgattagcctgaacattgatgcgaaagataaactggtgccactggtggaagaatatatgagcctgtataaagaatattttagcctgcgtggcgcgggcaactatgcggtgaactatcaggcgattccgggcatgatgagcaaagtgtatggcatggaaagcggctttgaacctgtgagcagcggcccgtttagcgcgcatctgcatcgttttgtggatgcggaacgttgggcggaacagtgcaccctgctgacccatgcggtgttttgcaaaattccggtgcgtgattttattccgaccggtagcaagaaaaagcatagcgtggaatttaccagcattaaacgttggtttcagcatattagcgcgtttgtgattaacgcggtgctggtgcagaacaccccggaagaacgtgcggaagtgattagcttttatctgaaactgagcctggatgcaaagaaaatgatgaacgaaatgcagctgctgagcgataaaggctgccgtgaaatgaaccgtctgatgcgtaaaaccgcgaacccgagcatgccgtatattggcctgtatctgcagggctttgtgggcctgaacgaactgccggcgtttgataaagaaggcctggtgaacgcgaaccgtctgcgtcgtatgggcgaactggcgatggaaattctgcatcgtcagagcgtggcgtataccctgcagcatgatgaagatgtgaacaaactgctgcatgtgagcctgccgtatagcagcgaagaaagccgttatgcgcgtagcctggaactggaaccgcgtgaagcggatgcgattccgctgagcgatcgtggcagctataccctgattgatgatgatgtggatctggaaaacgaagtgcgtgaaagcattggcggcgatggcacctttggctttcgtcagtggattcgtaaacagcaggtggtgcatcgtaaccgtagccgtagcagcgtgattgcgctgtatgaatgggtgggcggcggcgaattccatcatcatcatcatcatcatcatcatcatcattaagctt

SEQ ID NO: 16 - protein, 1065 aa, Artificial Sequence
  Derived from Phytophthora sojae
MASGWRQKPRLDPWDPPSPPPLLEEAAPKPKPKTSRFSRRSTRAKSHARQQAQHVELEVEEHAEQRDRDKRRSLLASMFGERAQSRASRSSSDASALQSQTQTLVQREKAFRLVRHGSDPLLNDAFYATQVQVGNFVKMGWLTKQGHMWKSWKTRFFVLFSDGTFAYYKNKGRKKIKGCMQLNDGVVSVQHVDIRLADKAYVFQIEKGFYKLLCYCCSQFEAELWVAALRSVRRVAPPCYEMDLTATEEKAGSNAVTRHLNKIFITDKQIAKRLAEFKANTHDHSYAAIHNFIVELDDAIIDRHHLEFYQDAEIELLPGNELVRLIRRHVEDRVFIPLYAEAYASLETDKVKTRRSNLEQHLKVLKQKTQADFGISKDLSVCNWKQAISVVNMLDCVSLPTHKFEVILSAGKAIMELIAQYNGELFEVSDEMLTAIFRYVVTMSSLSDLPTLRALLKYGYQHHPASQNKANVVTAFLNAIKWVECFEAGDESYQFDSLALAGSRVSVSISTNDVGIQFTTDGNGRGAIVYNIRKQSQAALSAAIVPGVADYCISLHNHDTLASILYALQSTAVQRLRKTIDCLSLIAINHEPVIALEFAVKIDYSHRIEVDKSASYLGSQMPNSSWKMVDKDDHVSRSARPGSVDDLIRELVAGTEVDLVDVVSFVLFLDSFSSLNEVVDRLSAHVRNGVKSHGLIRLFIVVLLFRQSETKEADDLRDRFYSLISLNIDAKDKLVPLVEEYMSLYKEYFSLRGAGNYAVNYQAIPGMMSKVYGMESGFEPVSSGPFSAHLHRFVDAERWAEQCTLLTHAVFCKIPVRDFIPTGSKKKHSVEFTSIKRWFQHISAFVINAVLVQNTPEERAEVISFYLKLSLDAKKMMNEMQLLSDKGCREMNRLMRKTANPSMPYIGLYLQGFVGLNELPAFDKEGLVNANRLRRMGELAMEILHRQSVAYTLQHDEDVNKLLHVSLPYSSEESRYARSLELEPREADAIPLSDRGSYTLIDDDVDLENEVRESIGGDGTFGFRQWIRKQQVVHRNRSRSSVIALYEWVGGGEFHHHHHHHHHHH

SEQ ID NO: 17 - protein, 267 aa, Artificial Sequence
  Derived from Phytophthora sojae
YQFDSLALAGSRVSVSISTNDVGIQFTTDGNGRGAIVYNIRKQSQAALSAAIVPGLSLIAINHEPVIALEFAVKIDYSHRIEVDKSASYLGSQMPNSSWKMVDKDDHVSRSARPGSVDDLIRELVAGTEVDLVDVVSFVLFLDSFSSLNEVVDRLSAHVRNGVKSHGLIRLFIVVLLFRQSETKEADDLRDRFYSLISLNIDAKDKLVPLVEEYMSLYKEYFSLRGAGNYAVNYQAIPGMMSKVYGMESGFEPVSSGPFSAHLHRFV

SEQ ID NO: 18 - protein, 450 aa, Artificial Sequence
  Derived from Phytophthora sojae
YQFDSLALAGSRVSVSISTNDVGIQFTTDGNGRGAIVYNIRKQSQAALSAAIVPGLSLIAINHEPVIALEFAVKIDYSHRIEVDKSASYLGSQMPNSSWKMVDKDDHVSRSARPGSVDDLIRELVAGTEVDLVDVVSFVLFLDSFSSLNEVVDRLSAHVRNGVKSHGLIRLFIVVLLFRQSETKEADDLRDRFYSLISLNIDAKDKLVPLVEEYMSLYKEYFSLRGAGNYAVNYQAIPGMMSKVYGMESGFEPVSSGPFSAHLHRFVHPFIQRNHAATATVTMHHLLFTNPLTKAKGAIAANAFTHFVFQIHIIINQGIAATIAQRNRIRFTRFQFQATRITAFFAAIRQAHMQQFVHIFIMLQGIRHALTMQNFHRQFAHTTQTVRVHQAFFIKRRQFVQAHKALQIQANIRHARVRGFTHQTVHFTAAFIAQQLHFVHHFLCIQAQFQ

SEQ ID NO: 19 - protein, 522 aa, Artificial Sequence
  Derived from Phytophthora sojae
YQFDSLALAGSRVSVSISTNDVGIQFTTDGNGRGAIVYNIRKQSQAALSAAIVPGLSLIAINHEPVIALEFAVKIDYSHRIEVDKSASYLGSQMPNSSWKMVDKDDHVSRSARPGSVDDLIRELVAGTEVDLVDVVSFVLFLDSFSSLNEVVDRLSAHVRNGVKSHGLIRLFIVVLLFRQSETKEADDLRDRFYSLISLNIDAKDKLVPLVEEYMSLYKEYFSLRGAGNYAVNYQAIPGMMSKVYGMESGFEPVSSGPFSAHLHRFVHPFIQRNHAATATVTMHHLLFTNPLTKAKGAIAANAFTHFVFQIHIIINQGIAATIAQRNRIRFTRFQFQATRITAFFAAIRQAHMQQFVHIFIMLQGIRHALTMQNFHRQFAHTTQTVRVHQAFFIKRRQFVQAHKALQIQANIRHARVRGFTHQTVHFTAAFIAQQLHFVHHFLCIQAQFQIKANHFRTFFRGVLHQHRVNHKRANMLKPTFNAGKFHAMLFLATGRNKITHRNFAKHRMGQQGALFRPTFRI

SEQ ID NO: 20 - DNA, 18 bp, Artificial Sequence
  Oligonicleotide F910
cgatcattgatcgtcacc

SEQ ID NO: 21 - DNA, 19 bp, Artificial Sequence
  Oligonuclotide F1880
aatacgctatgctcgtggg

SEQ ID NO: 22 - DNA, 23 bp, Artificial Sequence
  Oligonucleotide F2920
gcttgaacgaagtagtggatcgc

SEQ ID NO: 23 - DNA, 20 bp, Artificial Sequence
  Oligonucleotide R2150
gagcacatgaacattaacgg

SEQ ID NO: 24 - DNA, 19 bp, Artificial Sequence
  Oligonucleotide R2940
taacaccgtttcggacgtg

SEQ ID NO: 25 - DNA, 20 bp, Artificial Sequence
  Oligonucleotide R4120
tatggcatgctcggattagc

SEQ ID NO: 26 - DNA, 20 bp, Artificial Sequence
  Oligonucleotide R4560
cgacgtcaacatcgtcatcg

SEQ ID NO: 27 - DNA, 19 bp, Artificial Sequence
  Oligonucleotide R4708
cacccactcgtacagagct

SEQ ID NO: 28 - DNA, 19 bp, Artificial Sequence
  Oligonucleotide EAG
gctatcagtttgatagcct

SEQ ID NO: 29 - DNA, 18 bp, Artificial Sequence
  Oligonucleotide AVA
gcagatacaggccaatat

SEQ ID NO: 30 - protein, 1030 aa, Artificial Sequence
  Derived from Phytophthora sojae
MASGWRQKPRLDPWDPPSPPPLLEEAAPKPKPKTSRFSRRSTRAKSHARQQAQHVELEVEEHAEQRDRDKRRSLLASMFGERAQSRASRSSSDASALQSQTQTLVQREKAFRLVRHGSDPLLNDAFYATQVQVGNFVKMGWLTKQGHMWKSWKTRFFVLFSDGTFAYYKNKGRKKIKGCMQLNDGVVSVQHVDIRLADKAYVFQIEKGFYKLLCYCCSQFEAELWVAALRSVRRVAPPCYEMDLTATEEKAGSNAVTRHLNKIFITDKQIAKRLAEFKANTHDHSYAAIHNFIVELDDAIIDRHHLEFYQDAEIELLPGNELVRLIRRHVEDRVFIPLYAEAYASLETDKVKTRRSNLEQHLKVLKQKTQADFGISKDLSVCNWKQAISVVNMLDCVSLPTHKFEVILSAGKAIMELIAQYNGELFEVSDEMLTAIFRYVVTMSSLSDLPTLRALLKYGYQHHPASQNKANVVTAFLNAIKWVECFEAGDESYQFDSLALAGSRVSVSISTNDVGIQFTTDGNGRGAIVYNIRKQSQAALSAAIVPGLSLIAINHEPVIALEFAVKIDYSHRIEVDKSASYLGSQMPNSSWKMVDKDDHVSRSARPGSVDDLIRELVAGTEVDLVDVVSFVLFLDSFSSLNEVVDRLSAHVRNGVKSHGLIRLFIVVLLFRQSETKEADDLRDRFYSLISLNIDAKDKLVPLVEEYMSLYKEYFSLRGAGNYAVNYQAIPGMMSKVYGMESGFEPVSSGPFSAHLHRFVDAERWAEQCTLLTHAVFCKIPVRDFIPTGSKKKHSVEFTSIKRWFQHISAFVINAVLVQNTPEERAEVISFYLKLSLDAKKMMNEMQLLSDKGCREMNRLMRKTANPSMPYIGLYLQGFVGLNELPAFDKEGLVNANRLRRMGELAMEILHRQSVAYTLQHDEDVNKLLHVSLPYSSEESRYARSLELEPREADAIPLSDRGSYTLIDDDVDLENEVRESIGGDGTFGFRQWIRKQQVVHRNRSRSSVIALYEWVGGGEFHHHHHHHHHHH

(19) United States
(12) Patent Application Publication
(10) Pub. No.: US 2014/0329787 A1
(43) Pub. Date: Nov. 6, 2014

(54) PHYTOPHTHORA PHOSPHOLIPASE C

(71) Applicant: Deakin University, Waum Ponds, Victoria (AU)

(72) Inventors: Dean Ian Phillips, Box Hill (AU); Peter Luke Beech, Fitzroy (AU)

(21) Appl. No.: 14/352,611

(22) PCT Filed: Oct. 17, 2012
(86) PCT No.: PCT/AU2012/001265

§ 371 Date: Apr. 17, 2014

(30) Foreign Application Priority Data
Oct. 17, 2011 (AU) 2011904284

(51) Int. Cl. C12N 9/16; G01N 33/92; C12P 9/00; C12Q 1/44; A01N 45/00; C12P 7/64

(52) U.S. Cl. CPC C12N 9/16; A01N 45/00; C12P 7/649; C12P 7/6445; C12P 9/00; C12Q 1/44; G01N 33/92; C12Y 301/04003; G01N 2333/916; G01N 2500/02; G01N 2405/06
USPC 514/176; 536/23.2; 435/196; 435/188; 435/134; 435/131; 435/6.13; 435/19; 435/7.1; 506/11; 435/7.4

(57) ABSTRACT

The present invention relates to a novel Phytophthora phospholipase C and uses thereof, methods of identifying modulators and inhibitors of a biological function of the phospholipase C, and methods of inhibiting Phytophthora growth comprising inhibiting a biological function of a novel Phytophthora phospholipase C.

### FIELD OF THE INVENTION

The present invention relates to a novel Phytophthora protein and uses thereof, methods of identifying modulators and inhibitors of a biological function of the novel protein, and methods of inhibiting Phytophthora growth comprising inhibiting a biological function of a novel Phytophthora protein.

### BACKGROUND

Economically important plants may be attacked by a diverse range of plant pathogens. Many of the resulting diseases are caused by oomycete pseudo fungi, such as late blight of potato or tomato, can be especially damaging.

Since the first recorded outbreak of Phytophthora in the 1840's, which lead to the Irish potato famine, various species of Phytophthora have become major agricultural and environmental pests worldwide. For example, Phytophthora infestans was the infective agent of the potato blight that caused the Irish potato famine. The soya bean root and stem rot agent, Phytophthora sojae, has also caused longstanding problems for the agricultural industry.

Other important Phytophthora diseases include; Phytophthora alni which causes aider root rot; Phytophthora cactorum which causes rhododendron root rot affecting rhododendrons, azaleas and causes bleeding canker in hardwood trees; Phytophthora capsici which infects Cucurbitaceae fruits, such as cucumbers and squash; Phytophthora cinnamomi which causes cinnamon root rot affecting woody ornamentals including arborvitae, azalea, Chamaecyparis, dogwood, forsythia, Fraser fir, hemlock, Japanese holly, juniper, Pieris, rhododendron, Taxus, white pine, American chestnut and Australian Jarrah; Phytophthora fragariae which causes red root rot affecting strawberries; Phytophthora kernoviae, a pathogen of beech and rhododendron, also occurring on other trees and shrubs including oak, and holm oak; Phytophthora palmivora which causes fruit rot in coconuts and betel nuts; Phytophthora ramorum which causes Sudden Oak Death and infects over 60 plant genera and over 100 host species; Phytophthora quercina which causes oak death; and Phytophthora sojae which causes soybean root rot.

Currently, chemical control methods for the disease are limited to fungistatic agents but the diversity of Phytophthora strains within infection sites has led to the emergence of resistance. Furthermore, because of the difficulty of chemical control of Phytophthora, the growth of resistant cultivars is at present the main management strategy.

A need therefore exists for new compositions and methods for the prevention and/or treatment of Phytophthora.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or group thereof.

### SUMMARY OF INVENTION

The present invention is related in part to the Applicant's characterisation of an alternative phospholipase C (Alt-PLC) of the oomycete plant pathogen, Phytophthora. Applicant has demonstrated that Phytophthora has surprisingly independently evolved a phospholipase C (PLC) that shows no sequence homology to classical PLC proteins. The characterisation of this Alt-PLC, apparently restricted to the genus Phytophthora, presents an ideal target for antibiotic development.

Accordingly, in an aspect of the present invention there is provided an isolated, synthetic or recombinant nucleic acid (polynucleotide), wherein the nucleic acid comprises: (a) a nucleic acid sequence encoding a polypeptide having a phospholipase C enzyme activity, and (i) wherein the polypeptide has a phospholipase C enzyme activity in a Phytophthora; or (ii) having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or 100% sequence identity to of any one of SEQ ID NO: 1, SEQ ID NO: 2, SEQ ID NO: 3, SEQ ID NO: 7, SEQ ID NO: 8, SEQ ID NO: 9, SEQ ID NO: 10, SEQ ID NO: 11, SEQ ID NO: 13, or SEQ ID NO: 15; or (iii) encoding a polypeptide having an amino acid sequence as set forth in any one of SEQ ID NO: 4, SEQ ID NO: 5, SEQ ID NO: 6, SEQ ID NO: 12, SEQ ID NO: 14, SEQ ID NO: 16, SEQ ID NO: 17, SEQ ID NO: 18, SEQ ID NO: 19, or SEQ ID NO: 30; or (iv) hybridizes under stringent conditions to a nucleic acid comprising any one of SEQ ID NO: 1, SEQ ID NO: 7, SEQ ID NO:3, SEQ ID NO: 7, SEQ ID NO:8, SEQ ID NO:9, SEQ ID NO: 10, SEQ ID NO: 11, SEQ ID NO: 13, or SEQ ID NO: 15, wherein the stringent conditions comprise a wash step comprising a wash in 0.2×SSC at a temperature of about 65° C. for about 15 minutes; (b) a nucleic acid sequence according to (a) encoding a polypeptide having a phospholipase C enzyme activity but lacking a native promoter sequence; (c) a nucleic acid according to (b) further comprising a heterologous promoter sequence or other transcriptional regulatory sequence; (d) a nucleic acid sequence according to any one of (a) to (c) further comprising nucleic acid encoding a heterologous amino acid sequence, or further comprising a heterologous nucleotide sequence; (e) a nucleic acid according to (d), wherein the nucleic acid encoding the heterologous amino acid sequence comprises, or consists of, a sequence encoding a heterologous (leader) signal sequence, or a tag or an epitope, or the heterologous nucleotide sequence comprises a heterologous promoter sequence; (f) a nucleic acid according to (d) or (e), wherein the heterologous promoter sequence comprises or consists of a constitutive or inducible promoter, or a cell type specific promoter, or a plant specific promoter, or a bacteria specific promoter; (g) a nucleic acid sequence encoding a Phytophthora polypeptide having a phospholipase C enzyme activity; or (h) a nucleic acid sequence completely complementary to the nucleotide sequence of any one of (a) to (g).

In another aspect of the present invention there is provided a nucleic acid probe or amplification primer for isolating, making and/or identifying a nucleic acid encoding a polypeptide having a phospholipase C enzyme activity, wherein the probe comprises a nucleic acid as described herein.

In a further aspect of the present invention there is provided a vector, expression cassette, expression vector, plasmid, or cloning vehicle: (a) comprising a nucleic acid sequence as described herein; or, (b) a vector, expression cassette, expression vector, plasmid, or cloning vehicle of (a) comprising or contained in a viral vector, a phage, a phagemid, a cosmid, a fosmid, a bacteriophage, an artificial chromosome, an adenovirus vector, a retroviral vector or an adeno-associated viral vector; or, a bacterial artificial chromosome (BAC), a bacteriophage PI-derived vector (PAC), a yeast artificial chromosome (YAC), or a mammalian artificial chromosome (MAC).

In a further aspect of the present invention there is provided a host cell or a transformed cell: (a) comprising a nucleic acid sequence as described herein, or a vector, expression cassette, expression vector, plasmid, or cloning vehicle as described herein; or, (b) a host cell or a transformed cell according to (a), wherein the cell is a bacterial cell, a mammalian cell, a fungal cell, a yeast cell, an insect cell or a plant cell.

In a further aspect of the present invention there is provided a method of producing a variant nucleic acid encoding a phospholipase C enzyme activity, said method comprising; (a) providing a nucleic acid as described herein; (b) modifying, deleting or adding one or more nucleotides in the nucleic acid sequence of the nucleic acid of (a), or a combination thereof, to generate a variant nucleic acid of the nucleic acid of step (a).

In a further aspect of the present invention there is provided an isolated, synthetic or recombinant polypeptide having a phospholipase C enzyme activity, wherein the polypeptide comprises: (a) an amino acid sequence: (i) wherein the polypeptide has a phospholipase C enzyme activity in a Phytophthora; (ii) having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 5 97%, 98%, 99%, or more, or 100% sequence identity to any one of SEQ ID NO: 4, SEQ ID NO: 5, SEQ ID NO: 6, SEQ ID NO: 12, SEQ ID NO: 14, SEQ ID NO: 16, SEQ ID NO: 17, SEQ ID NO: 18, SEQ ID NO: 19, or SEQ ID NO: 30; or (iii) encoded by a nucleic acid as described herein; (b) a Phytophthora polypeptide having a phospholipase C enzyme activity; (c) a polypeptide according to (a) or (b) further comprising a heterologous amino acid sequence or a heterologous moiety; (d) a polypeptide according to (c), wherein the heterologous amino acid sequence or heterologous moiety comprises, or consists of a heterologous (leader) signal sequence, a tag, a detectable label or an epitope; (e) a polypeptide according to any one of (a) to (d), wherein the phospholipase C catalyzes a reaction comprising: PIP2+H2O [symbol] IP3+diacylglycerol and/or PIP2+H2O [symbol] IP3+monoacylglycerol; or (f) the polypeptide according to any one of (a) to (e), wherein: (i) the polypeptide is glycosylated, or the polypeptide comprises at least one glycosylation site, (ii) the polypeptide of (i) wherein the glycosylation is an N-linked glycosylation or an O-linked glycosylation; (iii) the polypeptide of (i) or (ii) wherein the polypeptide is glycosylated after being expressed in a yeast cell; or (iv) the polypeptide of (iii) wherein the yeast cell is a P. pastoris or a S. pombe.

In a further aspect of the present invention there is provided a protein preparation comprising the polypeptide as described herein, wherein the protein preparation comprises a liquid, a solid or a gel.

In a further aspect of the present invention there is provided a method for producing diacylglycerol comprising: (a) providing a phospholipase C enzyme, wherein the enzyme comprises a polypeptide as described herein, or a polypeptide encoded by a nucleic acid as described herein; (b) providing PIP₂; and (c) exposing the enzyme of step (a) to the PIP₂ of step (b) under conditions that facilitate an enzymatic reaction catalyzed by the phospholipase C enzyme, thereby producing diacylglycerol by a phospholipase C enzyme activity.

In a further aspect of the present invention there is provided a method for producing monoacylglycerol comprising: (a) providing a phospholipase C enzyme, wherein the enzyme comprises a polypeptide as described herein, or a polypeptide encoded by a nucleic acid according as described herein; (b) providing PIP₂; and (c) exposing the enzyme of step (a) to the PIP₂ of step (b) under conditions that facilitate an enzymatic reaction catalyzed by the phospholipase C enzyme, thereby producing monoacylglycerol by a phospholipase C enzyme activity.

In a further aspect of the present invention there is provided a method for producing free fatty acid comprising: (a) providing a phospholipase C enzyme, wherein the enzyme comprises a polypeptide as described herein, or a polypeptide encoded by the nucleic acid as described herein; (b) providing PIP₂; and (c) exposing the enzyme of step (a) to the PIP₂ of step (b) under conditions that facilitate an enzymatic reaction catalyzed by the phospholipase C enzyme, thereby monoacylglycerol and free fatty acid by a phospholipase C enzyme activity.

In a further aspect of the present invention there is provided a method for producing IP₃ comprising: (a) providing a phospholipase enzyme, wherein the enzyme comprises a polypeptide as described herein; or a polypeptide encoded by the nucleic acid as described herein; (b) providing PIP₂; and (c) exposing the enzyme of step (a) to the PIP₂ of step (b) under conditions that facilitate an enzymatic reaction catalyzed by the phospholipase enzyme, thereby producing IP₃ by a phospholipase C enzyme activity.

In a further aspect of the present invention there is provided a method for identifying a compound capable of modulating a phospholipase C enzyme activity, the method comprising: (a) providing a candidate compound; (b) providing (i) a polynucleotide as described herein 1; or (ii) a polypeptide according as described herein; (c) exposing the candidate compound to the polynucleotide or polypeptide; and (d) determining the level of a phospholipase C enzyme activity.

In one embodiment, a change in a level of a phospholipase C enzyme activity measured in the presence of the candidate compound compared to the activity in the absence of the candidate compound indicate that the test compound modulates a phospholipase C enzyme activity.

In another embodiment, a phospholipase C enzyme activity is measured by providing a phospholipase C substrate and detecting a decrease in the amount of the substrate or an increase in the amount of a reaction product, or, an increase in the amount of the substrate or a decrease in the amount of a reaction product.

In a further aspect of the present invention there is provided a method for identifying a compound capable of inhibiting a phospholipase C enzyme activity, the method comprising: (a) providing a candidate compound; (b) providing (i) a polynucleotide as described herein; or (ii) a polypeptide as described herein; (c) exposing the candidate compound to the polynucleotide or polypeptide; and (d) determining the level of inhibition of a phospholipase C enzyme activity.

In one embodiment, a decrease in a level of a phospholipase C activity measured in the presence of the candidate compound compared to the activity in the absence of the candidate compound indicates that the test compound inhibits a phospholipase C enzyme activity. In another embodiment, a decrease in the amount of a substrate or an increase in the amount of a reaction product with the candidate compound as compared to the amount of substrate or reaction product without the candidate compound indicates that the test compound is an activator of a phospholipase C enzyme activity. In another embodiment, an increase in the amount of a substrate or a decrease in the amount of a reaction product with the candidate compound as compared to the amount of substrate or reaction product without the candidate compound indicates that the test compound is an inhibitor of a phospholipase C enzyme activity.

In a further aspect of the present invention there is provided a method for determining whether a compound specifically binds to a polypeptide comprising: (a) providing a polypeptide as described herein; (b) providing a candidate compound; (c) exposing the polypeptide to the candidate compound; and (d) determining whether the test compound of step (b) specifically binds to the polypeptide.

Applicant has characterised accumulation of PIP₂, a phospholipase substrate of a phospholipase C activity reaction, when Phytophthora is treated the presence of an inhibitor.

Accordingly, in a further aspect of the present invention there is provided a method of inhibiting Phytophthora growth comprising contacting Phytophthora with a composition comprising an inhibitor of a Phytophthora phospholipase C enzyme activity.

In a further aspect of the present invention there is provided a method of preventing and/or treating infection of plants with Phytophthora comprising contacting a plant with a composition comprising an inhibitor of a Phytophthora phospholipase C enzyme activity.

In a further aspect of the present invention there is provided a method of controlling growth of Phytophthora in crops of cultivated plants comprising contacting Phytophthora with a composition comprising an inhibitor of a Phytophthora phospholipase C enzyme activity.

In a further aspect of the present invention there is provided a method of improving Phytophthora-sensitive plant growth comprising contacting the plant with a composition comprising an inhibitor of a Phytophthora phospholipase C enzyme activity.

In a further aspect of the present invention there is provided a composition comprising an inhibitor of a Phytophthora phospholipase C enzyme activity when used for inhibiting Phytophthora growth.

In a further aspect of the present invention there is provided a composition comprising an inhibitor of a Phytophthora phospholipase C enzyme activity when used for preventing Phytophthora growth.

Other aspects of the present invention will become apparent to those ordinarily skilled in the art upon review of the following description of specific embodiments of the invention.

### BRIEF DESCRIPTION OF THE DRAWINGS

For a further understanding of the aspects and advantages of the present invention, reference should be made to the following detailed description, taken in conjunction with the accompanying drawings.

FIG. 1 shows a comparison of Alt-PLC domain structure with classical phospholipase C (PLC) protein. There are three clear differences between classical PLC and Alt-PLC: (1) Alt-PLC uses RAS rather than Go-proteins for activation. (2) The TIM barrel in PLC is divided into two segments separated by a hyper-variable loop which come together in tertiary space; Alt-PLC's TIM barrel is not segregated in such a manner. (3) PLC also contains EF-Hand domains which provide scaffolding between the PH and catalytic domains. Without wishing to be bound by theory, Applicant believes that the VPS9 domain plays this role in Alt-PLC.

FIG. 2 shows MS analysis of Alt-PLC-PIP₂ hydrolysis. (A) The negative ion mass spectrum of the aqueous phase Alt-PLC hydrolyzing 5 mg of PI(4,5)P₂ yielded large quantities of inositol triphosphate [M-H]⁻ ion m/z. 160.6. (B) Positive ion ESI-MS analysis of neutral lipid species from hexane/methanol-H₂O the aqueous phase shown revealed a product matching sodiated monoacylglycerol [M+Na]⁺ at m/z 241 inset shows the two chiral forms possible from Alt-PLC hydrolysis. (C) Analysis of the hexane phase with positive ion ESI-MS revealed an unusual formation, where a free fatty acid covalently bonds with arginine in the reaction buffer forming acylated argingine [M-H]⁻ ion m/z. 301.1

FIG. 3 shows TLC analysis of acylglycerol products from Alt-PLC-PIP₂ hydrolysis. This analysis revealed a band of identical Rf to DAG control though an additional band at Rf=0.06 was also present which could not be identified by TLC. Lane 1: phosphatic acid control. Lane 2: diacylglycerol control. Lane 3: 10 μl acylglycerol sample produced from Alt-PLC-PIP₂ hydrolysis. Lane 4: 20 μl acylglycerol sample produced from Alt-PLC-PIP₂ hydrolysis

FIG. 4 shows ¹H NMR spectrum of the putative monoacylglycerol produced by Alt-PLC-driven hydrolysis of PI(4,5)P₂, demonstrating that Alt-PLC cleaves the acyl chain from the 2-carbon position yielding 1-MAG.

FIG. 5 shows a scheme of Alt-PLC's catalytic function on PI(4,5)P₂ in the presence of calcium. This reaction yields inositol(1,4,5)triphosphate, 1-monoacylglycerol and free fatty acid as the end products.

FIG. 6 shows a layout of codon-optimised Alt-PLC as constructed by Genescript.

FIG. 7 shows accumulation over time of the PLC substrate, PIP₂, when P. cinnamomi was grown in the presence of the inhibitor, U-73122. The hyphae were labelled with anti-PIP₂ antibody conjugated to FITC (green). Under normal (control) growth conditions, PIP₂ localised to regions of membrane deformation. However, when the PLC inhibitor was introduced, PIP₂ showed an almost uniform membrane distribution, indicating uncontrolled accumulation. Scale bar=50 μM.

FIG. 8 shows an alignment of the Alt-PLC homologs from P. infestans (_{—}03318.1), P. ramorum (PR_{—}94409), and P. sojae (S_{—}134249) by clustal X. This alignment shows high homology across all PIP₂ hydrolytic domains. However, Alt-PLC from both P. infestans and P. ramorum appear to have an additional domain inserted between the TIM barrel and the RAS-activating domain. The addition of domains from one species to another is not without precedence in the phospholipase C family-domain rearrangements being a common theme in classical PLCs.

FIG. 9 shows purification of Alt-PLC by high-pressure liquid chromatography using nickel-affinity purification. (A) shows the chromatogram of Ni affinity purification; arrow indicates elution position of Alt-PLC. (B) SDS-PAGE gel of Ni affinity purification. Lane 1=flow-through; lanes 2, 3=wash out; lane 4=Alt-PLC elution which removes all but two contaminating proteins. (C) SEC chromatogram comparing EDTA and β-mercaptoethanol in separating Alt-PLC from contaminants. (D) SDS-PAGE analysis of SEC. Lane 1=Ni affinity purified Alt-PLC. Lane 2 & 3=fractions A7 & A8 respectively eluted with β-mercaptoethanol. Lanes 4 & 5=fractions A7 & A8 eluted with EDTA. This analysis showed that Alt-PLC purified by SEC in the presence of b-mercaptoethanol yielded higher purity compared to EDTA and other buffer contents (not shown) (E) lane 1=Western blot with anti-his×6 antibody of E. coli/lysate expressing Alt-PLC confirmed the size of the target protein.

FIG. 10 shows an analysis of phosphate released following hydrolysis by Alt-PLC. (A) shows the phosphate (in PPM) in control, Alt-PLC and Alt-PLC+ calcium using Alt-PLC at 0.2 mg/ml and the same reactions using Alt-PLC at 0.5 mg/ml. (B) Standard curve of absorbance A₇₅₀ versus PO₄ concentration. Samples of known concentration were used at 1.25, 2.5, 5 & 10 ppm and measured in duplicate. And exponential trend line showed the best fit based on R² values.

FIG. 11 shows negative ion ESI-MS spectra of inositols produced by Alt-PLC. (Panels A, C, E) Ca²⁺-activated aqueous phase shows the emergence of peaks at m/z 160.6, which is the [M-H]⁻ of inositol triphosphate produced by Alt-PLC batches 1, 2 & 3 respectively. (Panels B, D, F) show the same protein batches inhibited by EDTA; note that no inositol triphosphate was observed.

FIG. 12 shows ¹H-NMR spectra of PIP₂ before and after hydrolysis with Alt-PLC. (A) shows the entire ¹H spectra of PI(4,5)P₂ before hydrolysis with inset showing the region containing glycerol and inositol resonances. (B) shows the ¹H spectra of the monoacylglycerol following hydrolysis and purification. Letters indicate proton assignments correlating with scheme inset.

FIG. 13 shows connectivity of 1-MAG by homonuclear irradiation. The resonance at 3.969 ppm was assigned to the b′ proton following a homonuclear decoupling experiment. This spectrum shows the attenuation of resonances at 3.969 ppm (using a 36 db attenuation) clearly causes the collapse of resonances at 4.17 ppm and 3.62 ppm.

FIG. 14 shows irradiation of the remaining unassigned resonances. (A) Irradiation of the doublet resonance at 4.07 ppm. (B) Irradiation of the resonance at 3.87 ppm. These attenuations showed that the unassigned resonances did not couple to one another or to other resonances within the spectra. We propose that these resonances represent 1,2 dimethoxyglycerol, which is a byproduct of purification. Arrows indicate attenuation pulse position.

FIG. 15 shows a chemical-transformation scheme for the production of 1,2-dimethoxyglycerol. We propose that 1,2 diacylglycerol remaining from incomplete hydrolysis may have undergone transformation to 1,2-dinmethoxyglycerol in the presence of methanol and HCl.

FIG. 16 shows a scheme of Alt-PLC catalytic function on inositol(1,4,5)P₃ nitrophenol in the presence of calcium. This reaction yields inositol(1,4,5)triphosphate and nitrophenol (Amax=405 nm) as the end products.

FIG. 17 shows a chemical-transformation scheme for the production of inositol(1,4,5)P₃ nitrophenol.

FIG. 18 shows an intron exon map of Alt-PLC from automated genome annotation and oligonucleotide primers used in determining intron/exon structure. A) Intron exon boundaries defined by automated annotation of the Alt-PLC gene were reconstructed by alignment using Sequencher. Intron 7 was annotated at 261 bp. B) Contig alignment with the position of primers utilised in resequencing and annotating the transcript.

FIG. 19 shows the results of reverse transcriptase PCR spanning introns 2-9 of Alt-PLC2. Lanes 1-3; amplification with primer pair A, lanes 4-6 amplification with primer pair B and lanes 7-9 amplification with primer pair C, as described in Table 1. Lanes 1, 4 and 7 are reactions using RNA as a negative control. Lanes 2, 5 and 8 are reactions using gDNA as template. Lanes 3, 6 and 9 use cDNA and thus represent the size of the Alt-PLC mRNA transcript. Bands in lane 3 and 9 were the size predicted by the data represented in FIG. 18 (above), however, lane 6 (primer pair B) yielded a fragment of approximately 800 bp, which was larger than the predicted size of 658 bp. Note ladder used is in increments of 100 bp. This indicates Alt-PLC2 includes a nucleotide sequence not in the automated annotation Alt-PLC shown in FIG. 18, above.

FIG. 20 shows Clustal-X alignment of the Alt-PLC-pProEX HTb protein sequence with a region of the consensus transcript from P. sojae UQ310 determined by re-sequencing. Only the sequence amplified by oligonucleotides F1880 and R4560 was translated and aligned in this figure (SEQ ID NO: 11). This clearly shows the 35aa insertion (SEQ ID NO: 12) at position 835aa in Alt-PLC2 (highlighted yellow). The locations of the TIM and RAS-GEF domains as determined by Expasy Prosite are highlighted in red and blue shading respectively. This shows that the 35aa insert is not predicted to affect the functionality of the catalytic barrel.

FIG. 21 shows the psi-pred result on the additional (relative to Alt-PLC) 35aa sequence within Alt-PLC2. This indicated, with a high confidence interval, that the sequence has a helical structure.

FIG. 22 shows a Taq screen of E. coli colonies transformed with the codon-optimised Alt-PLC2. Alt-PLC-pProEx HTb (codon optimised Alt-PLC) was used as a control in lane 1 with an expected amplified product size of 1100 bp. Lanes 2-14 are colonies transformed with Alt-PLC2 with an expected amplified product size 105 bp larger than Alt-PLC-pProEX HTb. All 13 transformants contain the new fragment ligated into the Eagl and Avall RE-sites. This figure shows the generation of codon optimised Alt-PLC2.

FIG. 23 shows expression of Alt-PLC2 protein in vivo. Colonies 1-6 were used for expression profiling in 2 ml LB cultures (lanes 2-7), and compared to the uninduced control (lane 1). The E. coli expressed Alt-PLC2 ran at an apparent molecular weight of 130 kDa.

FIG. 24 shows Histrap purification of Alt-PLC2 and SEC chromatograms. A) Double Histrap purification of Alt-PLC2 shows increased yield and reduced non-specific binding compared to Alt-PLC as shown in FIG. 9A. B) Size exclusion purification of Alt-PLC2 (second dimension), showing ideal resolution/separation from contaminants (arrows indicate Alt-PLC2 elution peak positions).

FIG. 25 shows an alignment of the synthetic Alt-PLC-pProEx HTb with Alt-PLC (Ps-248481). The upper sequence is from the P. sojae genome and the lower from the synthetic Alt-PLC gene. The only difference present is the poly-histidine tag and linker on the C-termini of the protein encoded by the synthetic gene. Thus there was no error in the synthetic Alt-PLC construct.

FIG. 26 shows enzymatic activity of Alt-PLC2. Thin layer chromatography of Alt-PLC and Alt-PLC2 hydrolysis of PIPx demonstrates both Alt-PLC and Alt-PLC2 hydrolyse PIPx and produce monoacylglycerol.

FIG. 27 shows derivatives of Alt-PLC generated for in vivo expression and enzymatic activity assays. The truncations of Alt-PLC generated are represented schematically by the blue lines. The N-terminus was removed in the each derivative due to the higher hydrophobicity in this region. The amino acid sequence of t1 is shown in SEQ ID NO: 17, The amino acid sequence of t2 is shown in SEQ ID NO: 18. The amino acid sequence of t3 is shown in SEQ ID NO: 19.

FIG. 28 shows growth curves of E. coli expressing truncated derivatives of Alt-PLC. This graph shows expression of Alt-PLC reduces the growth rate of E. coli expressing Alt-PLC relative to wild-type E. coli (BL21) (not expressing Alt-PLC), and empty vector controls (c). Truncations 1 and 2 (t1, t2) have similar growth to (full-length) Alt-PLC. Truncation 3 however (t3), showed significantly reduced growth than E. coli expressing Alt-PLC. This suggests that this derivative (t3) has a higher activity and possible broader substrate range than Alt-PLC.

FIG. 29 shows the detection and quantification of Free Fatty acids generated from Alt-PLC enzyme activity with PI(4,5)P₂ samples.

### DETAILED DESCRIPTION OF THE INVENTION

Since the first recorded outbreak of Phytophthora in the 1840's, which lead to the Irish potato famine, various species of Phytophthora have become major agricultural and environmental pests worldwide. Currently, chemical control methods for the disease are limited to fungistatic agents but the diversity of Phytophthora strains within infection sites has led to the emergence of resistance in 2006, the genomes of P. sojae and P. ramorum were sequenced and the apparent lack of a gene for phospholipase C was noted—a feature that appears to be consistent across the genus and unique in biology.

The enzyme, PLC, establishes a number of key cellular signalling cascades that lead to, for example, cytokinesis. When activated by G proteins (which are themselves activated by G protein-coupled receptors that sense the external environment), PLC hydrolyzes the phospholipid, phosphatidyl inositol(4,5)bisphosphate (PIP₂), into the secondary products (1,4,5) inositol triphosphate (IP₃) and (1,2)diacylglycerol (DAG). When IP₃ is released into the cytoplasm, it activates a host of secondary proteins, such as protein kinase C and various transcription factors, as well as Ca²⁺ channels in the endoplasmic reticulum. Activation of these channels results in rapid calcium flux and is recognized as the critical factor in the initiation of cytokinesis. Given the importance of PLC in cellular signalling, its conspicuous absence in Phytophthora raised numerous questions about how this organism sensed and responded to the environment. Intriguingly, evidence suggests that Phytophthora species still utilize the PLC pathway. For example, zoosporogenesis in Phytophthora requires multinucleated sporangia to undergo cytokinesis and these cell divisions are initiated by a mechanism of rapid calcium flux similar to that generated by PLC.

The present invention is related in part to the Applicant's characterisation of an alternative phospholipase C (Alt-PLC) of the oomycete plant pathogen, Phytophthora. Applicant has demonstrated that Phytophthora has surprisingly independently evolved a phospholipase C (PLC) that shows no sequence homology to classical PLC proteins. The characterisation of this Alt-PLC in a number of Phytophthora species, apparently restricted to the genus Phytophthora, presents an ideal target for antibiotic development.

Accordingly, in an aspect of the present invention there is provided an isolated, synthetic or recombinant nucleic acid (polynucleotide), wherein the nucleic acid comprises: (a) a nucleic acid sequence encoding a polypeptide having a phospholipase C enzyme activity, and (i) wherein the polypeptide has a phospholipase C enzyme activity in a Phytophthora; or (ii) having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or 100% sequence identity to of any one of SEQ ID NO: 1, SEQ ID NO: 2, SEQ ID NO: 3, SEQ ID NO: 7, SEQ ID NO: 8, SEQ ID NO: 9, SEQ ID NO: 10, SEQ ID NO: 11, SEQ ID NO: 13, or SEQ ID NO: 15; or (iii) encoding a polypeptide having an amino acid sequence as set forth in any one of SEQ ID NO: 4, SEQ ID NO: 5, SEQ ID NO: 6, SEQ ID NO: 12, SEQ ID NO: 14, SEQ ID NO: 16, SEQ ID NO: 17, SEQ ID NO: 18, SEQ ID NO: 19, or SEQ ID NO: 30; or (iv) hybridizes under stringent conditions to a nucleic acid comprising any one of SEQ ID NO: 1, SEQ ID NO: 2, SEQ ID NO: 3, SEQ ID NO: 7, SEQ ID NO: 8, SEQ ID NO: 9, SEQ ID NO: 10, SEQ ID NO: 11, SEQ ID NO: 13, or SEQ ID NO: 15, wherein the stringent conditions comprise a wash step comprising a wash in 0.2×SSC at a temperature of about 65° C. for about 15 minutes; (b) a nucleic acid sequence according to (a) encoding a polypeptide having a phospholipase C enzyme activity but lacking a native promoter sequence; (c) a nucleic acid according to (b) further comprising a heterologous promoter sequence or other transcriptional regulatory sequence; (d) a nucleic acid sequence according to any one of (a) to (c) further comprising nucleic acid encoding a heterologous amino acid sequence, or further comprising a heterologous nucleotide sequence: (e) a nucleic acid according to (d), wherein the nucleic acid encoding the heterologous amino acid sequence comprises, or consists of, a sequence encoding a heterologous (leader) signal sequence, or a tag or an epitope, or the heterologous nucleotide sequence comprises a heterologous promoter sequence; (f) a nucleic acid according to (d) or (e), wherein the heterologous promoter sequence comprises or consists of a constitutive or inducible promoter, or a cell type specific promoter, or a plant specific promoter, or a bacteria specific promoter; (g) a nucleic acid sequence encoding a Phytophthora polypeptide having a phospholipase C enzyme activity; or (h) a nucleic acid sequence completely complementary to the nucleotide sequence of any one of (a) to (g).

The nucleotide sequence of a mRNA transcript encoding Phytophthora ramorum Alt-PLC1 is shown in SEQ ID NO: 1. The nucleotide sequence of a mRNA transcript encoding Phytophthora sojae Alt-PLC1 is shown in SEQ ID NO: 2. The nucleotide sequence of a mRNA transcript encoding Phytophthora infestans Alt-PLC1 is shown in SEQ ID NO: 3. The amino acid sequence of Phytophthora ramorum Alt-PLC enzyme is shown in SEQ ID NO: 4.

The amino acid sequence of the Phytophthora sojae Alt-PLC1 enzyme is shown in SEQ ID NO: 5. The amino acid sequence of the Phytophthora infestans Alt-PLC1 enzyme is shown in SEQ ID NO: 6. The nucleotide sequence of the genomic region encoding Phytophthora ramorum Alt-PLC1 is shown in SEQ ID NO: 7. The nucleotide sequence of the genomic region encoding Phytophthora sojae Alt-PLC1 is shown in SEQ ID NO: 8. The nucleotide sequence of the genomic region encoding Phytophthora infestans Alt-PLC1 is shown in SEQ ID NO: 9. The nucleotide sequence of a codon optimised Phytophthora sojae Alt-PLC1 is shown in SEQ ID NO: 10. The nucleotide sequence of a portion of a cDNA transcript encoding Phytophthora sojae Alt-PLC2 is shown in SEQ ID NO: 11. The amino acid sequence of a portion of a Phytophthora sojae Alt-PLC2 not included in SEQ ID NO: 5 is shown in SEQ ID NO: 12. The nucleotide sequence of a mRNA transcript encoding Phytophthora sojae Alt-PLC2 is shown in SEQ ID NO: 13. The amino acid sequence of a Phytophthora sojae Alt-PLC2 is shown in SEQ ID NO: 14. The nucleotide sequence of a codon optimised Phytophthora sojae Alt-PLC2 is shown in SEQ ID NO: 15. The amino acid sequence of a codon optimised Phytophthora sojae Alt-PLC2 is shown in SEQ ID NO: 16. The amino acid sequence of a truncated Phytophthora sojae Alt-PLC1 (“t1”) is shown in SEQ ID NO: 17. The amino acid sequence of a truncated Phytophthora sojae Alt-PLC1 (“t2”) is shown in SEQ ID NO: 18. The amino acid sequence of a truncated Phytophthora sojae Alt-PLC1 (“t3”) is shown in SEQ ID NO: 19. The nucleotide sequence of oligonucleotide F910 is shown in SEQ ID NO: 20. The nucleotide sequence of oligonucleotide F1880 is shown in SEQ ID NO: 21. The nucleotide sequence of oligonucleotide F2920 is shown in SEQ ID NO: 22. The nucleotide sequence of oligonucleotide F2150 is shown in SEQ ID NO: 23. The nucleotide sequence of oligonucleotide F2940 is shown in SEQ ID NO: 24. The nucleotide sequence of oligonucleotide F4120 is shown in SEQ ID NO: 25. The nucleotide sequence of oligonucleotide F4560 is shown in SEQ ID NO: 26. The nucleotide sequence of oligonucleotide F4708 is shown in SEQ ID NO: 27. The nucleotide sequence of oligonucleotide EAG is shown in SEQ ID NO: 28. The nucleotide sequence of oligonucleotide AVA is shown in SEQ ID NO: 29. The amino acid sequence of a codon optimised Phytophthora sojae Alt-PLC1 is shown in SEQ ID NO: 30.

The phrase “enzyme activity” refers to the ability of an enzyme to catalyze the conversion of a substrate into a product. A substrate for the enzyme comprises the natural substrate of the enzyme, but can also comprise analogues of the natural substrate, which can also be converted, by the enzyme into a product or into an analogue of a product. The activity of the enzyme can be measured, for example, by determining the amount of product in the reaction after a certain period of time, or by determining the amount of substrate remaining in the reaction mixture after a certain period of time.

The term “a phospholipase C enzyme activity”, as used herein, includes an enzyme activity that cleaves phospholipids. In one embodiment, a phospholipase C enzyme activity includes cleavage of phosphatidylinositol 4,5-bisphosphate (PIP₂) to produce diacylglycerol (DAG). In another embodiment, a phospholipase C enzyme activity includes cleavage of phosphatidylinositol 4,5-bisphosphate (PIP₂) to produce monoacylglycerol (MAG). In another embodiment, a phospholipase C enzyme activity includes cleavage of phosphatidylinositol 4,5-bisphosphate (PIP₂) to produce free fatty acid (FFA).

The term “Alt-PLC”, as used herein, includes a Phytophthora polypeptide having a phospholipase C enzyme activity, a nucleotide encoding a Phytophthora polypeptide having a phospholipase C enzyme activity, a polypeptide having a phospholipase C enzyme activity in a Phytophthora species, or a nucleotide encoding a polypeptide having a phospholipase C enzyme activity in a Phytophthora species. The term includes Alt-PLC1 and Alt-PLC2, and variants, mutants and derivatives described herein.

The term “polynucleotide”, as used herein, includes DNA and RNA, and also their analogues, such as those containing modified backbones (e.g. phosphorothioates, etc.), and also peptide nucleic acids (PNA), etc. The invention includes nucleic acid comprising sequences complementary to those described above (e.g. for antisense or probing purposes). The skilled person understands that strict compliance with the polynucleotide and protein sequences defined herein is not necessary, and functional equivalents are included in the scope of the invention. Various strains and species of Phytophthora may have differences at various amino acid and/or nucleotide residues without substantially affecting a phospholipase C enzyme activity or structure of the protein. For example, in respect of proteins it is known that the certain amino acid substitutions can be made without substantially affecting the structure or function of the protein. Such “conservative substitutions” are well known to the skilled person and will not be repeated herein. It is also understood that a protein may be truncated, or have internal deletions without substantially affecting structure or function. Furthermore, certain fragments of a protein may retain important structure and function.

The degeneracy of the genetic code is such that the same protein may be encoded by a number of different polynucleotide sequences. The present invention includes any alterations that are available by virtue of the degeneracy of the genetic code. Furthermore, the invention provides nucleic acid which can hybridise to these nucleic acid molecules, preferably under “stringent” conditions (e.g. 65° C. in a 0.2×SSC). Nucleic acid according to the invention can be prepared in many ways (e.g. by chemical synthesis, from genomic or cDNA libraries, from the organism itself, etc.) and can take various forms (e.g. single stranded, double stranded, vectors, probes, etc.). They are preferably prepared in substantially pure form (i.e. substantially free from other Phytophthora or host cell nucleic acids).

Similarly, the skilled person understands that strict compliance with any amino acid sequence disclosed herein is not necessarily required, and he or she could decide by a matter of routine whether any further mutation is deleterious or preferred. For example, where the protein has a given biological activity that can be assayed (such a phospholipase C enzyme activity as described herein) the effect of any mutation on that biological activity may be directly observed. Thus, the polypeptides of the present invention include sequences having 50% or more identity (e.g. 60%, 65%, 70%, 75%, 80%, 85%, 90%, 91%, 92%, 93%, 94%, 95%, 96%, 97%, 98%, 99%, 99.5% or more) to any protein disclosed herein. The polypeptides also include variants (e.g. allelic variants, homnologs, orthologs, paralogs, mutants, etc.). The molecules may lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus.

Functional equivalents of the immunogenic proteins are included within the scope of the invention.

The term “polypeptide”, as used herein, includes to amino acid polymers of any length. The protein may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labelling component. Also included are, for example, proteins containing one or more analogs of an amino acid (including, for example, unnatural amino acids, etc.), as well as other modifications known in the art. Proteins can occur as single chains or associated chains.

Polypeptides of the invention can be prepared by various means (e.g. recombinant expression, purification from cell culture, chemical synthesis, etc.) and in various forms (e.g. native, fusions, non-glycosylated, lipidated, etc.). They are preferably prepared in substantially pure form (i.e. substantially free from other Phytophthora or host cell proteins).

The term “sequence identity”, as used herein, includes, in the context of two or more nucleic acids or polypeptide sequences, to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. Sequence identity, homology and the like may be determined using standard methods known the skilled person, for example, using any computer program and associated parameters, such as BLAST or FASTA.

The term “stringent conditions”, as used herein, includes highly stringent conditions, medium stringent conditions, low stringent conditions, including the high and reduced stringency conditions described herein. In alternative embodiments, nucleic acids of the invention as defined by their ability to hybridize under stringent conditions can be between about five residues and the full length of the molecule, e.g., an exemplary nucleic acid of the invention. For example, they can be at least 5, 10, 15, 20, 25, 30, 35, 40, 50, 55, 60, 65, 70, 75, 80, 90, 100, 150, 200, 250, 300, 350, 400 or more residues in length. Nucleic acids shorter than full length are also included. These nucleic acids are useful as, e.g., hybridization probes, labelling probes, PCR oligonucleotide probes, antisense or sequences encoding antibody binding peptides (epitopes), motifs, active sites, binding domains, regulatory domains and the like.

In one aspect, nucleic acids of the invention are defined by their ability to hybridize under high stringency comprises conditions of about 50% formamide at about 37° C. to 42° C. In one aspect, nucleic acids of the invention are defined by their ability to hybridize under reduced stringency comprising conditions in about 35% to 25% formamide at about 30° C. to 35° C.

Alternatively, nucleic acids of the invention are defined by their ability to hybridize under high stringency comprising conditions at 42° C. in 50% formamide, 5×SSPE, 0.3% SDS, and a repetitive sequence blocking nucleic acid, such as cot-1 or salmon sperm DNA (e.g., 200 ug/ml sheared and denatured salmon sperm DNA). In one aspect, nucleic acids of the invention are defined by their ability to hybridize under reduced stringency conditions comprising 35% formamide at a reduced temperature of 35° C.

Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

The temperature range corresponding to a particular level of stringency can be further narrowed by calculating the purine to pyrimidine ratio of the nucleic acid of interest and adjusting the temperature accordingly. Nucleic acids of the invention are also defined by their ability to hybridize under high, medium, and low stringency conditions as set forth in Ausubel and Sambrook. Variations on the above ranges and conditions can be used to practice the invention and are well known in the art.

The term “native promoter”, as used herein, includes a promoter that is endogenous to the organism or virus and is unmodified with respect to its nucleotide sequence and its position in the viral genome as compared to a wild-type organism or virus.

The term “heterologous promoter”, as used herein, includes a promoter that is not normally found in the wild-type organism or that is at a different locus as compared to a wild type organism. A heterologous promoter is often not endogenous to a cell or virus into which it is introduced, but has been obtained from another cell or virus or prepared synthetically. A heterologous promoter can refer to a promoter from another cell in the same organism or another organism, including the same species or another species. A heterologous promoter, however, can be endogenous, but is a promoter that is altered in its sequence or occurs at a different locus (e.g., at a different location in the genome or on a plasmid). Thus, a heterologous promoter includes a promoter not present in the exact orientation or position as the counterpart promoter is found in a genome. A synthetic promoter is a heterologous promoter that has a nucleotide sequence that is not found in nature. A synthetic promoter can be a nucleic acid molecule that has a synthetic sequence or a sequence derived from a native promoter or portion thereof. A synthetic promoter can also be a hybrid promoter composed of different elements derived from different native promoters.

A heterologous nucleic acid (also referred to as exogenous nucleic acid or foreign nucleic acid) includes a nucleic acid that is not normally produced in vivo by an organism from which it is expressed or that is produced by an organism but is at a different locus, expressed differently, or that mediates or encodes mediators that alter expression of endogenous nucleic acid, such as DNA, by affecting transcription, translation, or other regulatable biochemical processes.

Heterologous nucleic acid is often not endogenous to a cell or virus into which it is introduced, but has been obtained from another cell or prepared synthetically. Heterologous nucleic acid can refer to a nucleic acid molecule from another cell in the same organism or another organism, including the same species or another species. Heterologous nucleic acid, however, can be endogenous, but is nucleic acid that is expressed from a different locus or altered in its expression or sequence (e.g., a plasmid). Thus, heterologous nucleic acid includes a nucleic acid molecule not present in the exact orientation or position as the counterpart nucleic acid molecule, such as DNA, is found in a genome. Generally, although not necessarily, such nucleic acid encodes RNA and proteins that are not normally produced by the cell or virus or in the same way in the cell in which it is expressed. Any nucleic acid, such as DNA, that one of skill in the art recognizes or considers as heterologous, exogenous or foreign to the cell in which the nucleic acid is expressed is herein encompassed by heterologous nucleic acid.

The invention provides nucleic acid (e.g., DNA) sequences of the invention operatively linked to an expression regulatory sequence (including transcriptional regulatory sequence or translational regulatory sequence) e.g., promoters or enhancers, to direct or modulate RNA synthesis/expression. The expression control sequence can be in an expression vector. Exemplary bacterial promoters include lacI, lacZ, T3, T7, gpt, lambda PR, PL and trp. Exemplary eukaryotic promoters include CMV immediate early, HSV thymidine kinase, early and late SV40, LTRs from retrovirus, and mouse metallothionein I. In one embodiment the promoter is trc. In one embodiment, the expression control sequence is inducible.

Applicant has generated isolated and mutant variants of nucleic acids encoding Alt-PLC, and cloned and expressed Alt-PLC in heterologous cells.

In another aspect of the present invention there is provided a nucleic acid probe or amplification primer for isolating, making and/or identifying a nucleic acid encoding a polypeptide having a phospholipase C enzyme activity, wherein the probe comprises a nucleic acid as described herein.

In another aspect, provided herein are nucleic acid probes or amplification primers for isolating, making and/or identifying a nucleic acid encoding a polypeptide having a phospholipase C enzyme activity. In one embodiment, a nucleic acid probe, e.g., a probe for identifying a nucleic acid encoding a polypeptide having a phospholipase C enzyme activity, comprises a probe comprising or consisting of at least about 10, 15, 20, 25, 30, 35, 40, 45, 50, 55, 60, 65, 70, 75, 80, 85, 90, 95, 100, 150, 200 or more, consecutive bases of a sequence as provided herein, or fragments or subsequences thereof, wherein the probe identifies the nucleic acid by binding or hybridization. The probe can comprise an oligonucleotide comprising at least about 10 to 50, about 20 to 60, about 30 to 70, about 40 to 80, or about 60 to 100 consecutive bases of a sequence comprising a sequence as provided herein, or fragments or subsequences thereof. The probe can comprise an oligonucleotide comprising at least about 10 to 50, about 20 to 60, about 30 to 70, about 40 to 80, or about 60 to 100 consecutive bases of a nucleic acid sequence as provided herein, or a subsequence thereof.

In one embodiment, an amplification primer sequence pair for amplifying a nucleic acid encoding a polypeptide having a phospholipase C enzyme activity, comprises a primer pair comprising or consisting of a primer pair capable of amplifying a nucleic acid comprising a sequence as provided herein, or fragments or subsequences thereof. One or each member of the amplification primer sequence pair can comprise an oligonucleotide comprising at least about 10, 11, 12, 13, 14, 15, 16, 17, 18, 19, 20, 21, 22, 23, 24, or 25 consecutive bases of the sequence.

In one embodiment, methods of amplifying a nucleic acid encoding a polypeptide having a phospholipase C enzyme activity, comprise amplification of a template nucleic acid with an amplification primer sequence air capable of amplifying a nucleic acid sequence as provided herein, or fragments or subsequences thereof.

In a further aspect of the present invention there is provided a nucleic acid probe vector, expression cassette, expression vector, plasmid, or cloning vehicle: (a) comprising a nucleic acid sequence as described herein or, (b) a vector, expression cassette, expression vector, plasmid, or cloning vehicle of (a) comprising or contained in a viral vector, a phage, a phagemid, a cosmid, a fosmid, a bacteriophage, an artificial chromosome, an adenovirus vector, a retroviral vector or an adeno-associated viral vector; or, a bacterial artificial chromosome (BAC), a bacteriophage PI-derived vector (PAC), a yeast artificial chromosome (YAC), or a mammalian artificial chromosome (MAC).

In a further aspect of the present invention there is provided a host cell or a transformed cell: (a) comprising a nucleic acid sequence as described herein, or a vector, expression cassette, expression vector, plasmid, or cloning vehicle as described herein; or, (b) a host cell or a transformed cell according to (a), wherein the cell is a bacterial cell, a mammalian cell, a fungal cell, a yeast cell, an insect cell or a plant cell.

In one embodiment, expression cassettes comprise a nucleic acid as provided herein or a subsequence thereof.

In one aspect, the expression cassette can comprise a nucleic acid that is operably linked to a promoter. The promoter can be a viral, bacterial, mammalian or plant promoter. In one aspect, the plant promoter can be a potato, rice, corn, wheat, tobacco or barley promoter. The promoter can be a constitutive promoter. The constitutive promoter can comprise CaMV35S.

In another aspect, the promoter can be an inducible promoter. In one aspect, the promoter can be a tissue-specific promoter or an environmentally regulated or a developmentally regulated promoter. Thus, the promoter can be, e.g., a seed-specific, a leaf-specific, a root-specific, a stem-specific or an abscission-induced promoter. In one aspect, the expression cassette can further comprise a plant or plant virus expression vector.

In one embodiment, a host cell or a transformed cell comprises a nucleic acid as provided herein. In one aspect, the host cell or a transformed cell can be a bacterial cell, a mammalian cell, a fungal cell, a yeast cell, an insect cell or a plant cell. In one aspect, the plant cell can be a potato, wheat, rice, corn, tobacco or barley cell. The transformed cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include any species within the genera Escherichia, Bacillus, Streptomyces, Salmonella, Pseudomonas and Staphylococcus, including, e.g., Escherichia coli, Lactococcus lactis, Bacillus subtilis, Bacillus cereus, Salmonella typhimurium, Pseudomonas fluorescens. Exemplary fungal cells include any species of Aspergillus. Exemplary yeast cells include any species of Pichia, Saccharomyces, Schizosaccharomyces, or Schwanniomyces, including Pichia pastoris, Saccharomyces cerevisiae, or Schizosaccharomnyces pombe. Exemplary insect cells include any species of Spodoptera or Drosophila, including Drosophila S2 and Spodoptera Sf9. Exemplary animal cells include CHO, COS or Bowes melanoma or any mouse or human cell line.

In another embodiment, transgenic non-human animals comprise a nucleic acid as provided herein or a vector, expression cassette, expression vector, plasmid, or cloning vehicle as provided herein. The transgenic non-human animal can be a mouse, a rat, a goat, a rabbit, a sheep, a pig or a cow.

In one embodiment, a transgenic plant or seed comprises a nucleic acid as provided herein or a vector, expression cassette, expression vector, plasmid, or cloning vehicle as provided herein. In one embodiment, plant is a corn plant, a sorghum plant, a potato plant, a tomato plant, a wheat plant, an oilseed plant, a rapeseed plant, a soybean plant, a rice plant, a barley plant, a grass, a cottonseed, a palm, a sesame plant, a peanut plant, a sunflower plant or a tobacco plant; the transgenic seed. In one embodiment, the seed is a corn seed, a wheat kernel, an oilseed, a rapeseed, a soybean seed, a palm kernel, a sunflower seed, a sesame seed, a rice, a barley, a peanut, a cottonseed, a palm, a peanut, a sesame seed, a sunflower seed or a tobacco plant seed.

The nucleic acids of the invention can be used to confer desired traits on essentially any plant, e.g., on oil-seed containing plants, such as rice, soybeans, rapeseed, sunflower seeds, sesame and peanuts. Nucleic acids of the invention can be used to manipulate metabolic pathways of a plant in order to optimize or alter host's expression of phospholipase. The can change phospholipase activity in a plant. Alternatively, a phospholipase C enzyme of the invention can be used in production of a transgenic plant to produce a compound not naturally produced by that plant. This can lower production costs or create a novel product. Suitable methods of generating such plants are known to the skilled person.

In a further aspect of the present invention there is provided a method of producing a variant nucleic acid encoding a phospholipase C enzyme activity, said method comprising; (a) providing a nucleic acid as described herein; (b) modifying, deleting or adding one or more nucleotides in the nucleic acid sequence of the nucleic acid of (a), or a combination thereof, to generate a variant nucleic acid of the nucleic acid of step (a).

The invention provides methods of generating variants of the nucleic acids of the invention, e.g., those encoding a phospholipase C enzyme activity. In alternative embodiment, the invention provides methods for modifying an enzyme of the invention, e.g., by mutation of its coding sequence by random or stochastic methods, or, non-stochastic, or “directed evolution” such as Gene Site Saturation Mutagenesis™ (GSSM), to alter a characteristic of an enzyme described herein, for example, pH range of activity or range of optimal activity, temperature range of activity or range of optimal activity, specificity, activity (kinetics);

Applicant has generated variants of nucleic acids encoding a phospholipase C enzyme activity, including codon optimised nucleotides encoding a phospholipase C enzyme activity.

Accordingly, the invention provides methods for modifying an enzyme of the invention, e.g., by mutation of its coding sequence, e.g., by GSSM, to optimise codon usage for expression in a heterologous organism. The invention provides methods for modifying an enzyme of the invention.

In alternative embodiments, the invention provides variants of exemplary nucleic acids and polypeptides of the invention, including e.g., SEQ ID NOs: 1, 2, 3, 7, 8, 9, 10, 11, 13, or 15. In alternative embodiments, variants of polynucleotides or polypeptides of the invention are nucleic acids or polypeptides that have been modified at one or more base pairs, codons, introns, exons, or amino acid residues (respectively) yet still retain a phospholipase C enzyme activity. Variants can be produced by any number of means included methods such as, for example, error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, GSSM and any combination thereof. Techniques for producing variant phospholipase Cs having activity at a pH or temperature, for example, that is different from a wild-type phospholipase, are included herein.

These methods can be repeated or used in various combinations to generate phospholipase C enzymes having an altered or different activity or an altered or different stability from that of a phospholipase C encoded by the template nucleic acid. These methods also can be repeated or used in various combinations, e.g., to generate variations in gene/message expression, message translation or message stability. In another aspect, the genetic composition of a cell is altered by, e.g., modification of a homologous gene ex vivo, followed by its reinsertion into the cell.

A nucleic acid of the invention can be altered by any means. For example, random or stochastic methods, or, non-stochastic, or “directed evolution,” methods. Any technique in molecular biology can be used, e.g., random PCR mutagenesis, or combinatorial multiple cassette mutagenesis. Alternatively, nucleic acids, e.g., genes, can be reassembled after random, or “stochastic.” fragmentation. In alternative aspects, modifications, additions or deletions are introduced by error-prone PCR, shuffling, oligonucleotide-directed mutagenesis, assembly PCR, PCR mutagenesis, in vivo mutagenesis, cassette mutagenesis, recursive ensemble mutagenesis, exponential ensemble mutagenesis, site-specific mutagenesis, gene reassembly, Gene Site Saturation, Mutagenesis (GSSM), synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, and/or a combination of these and other methods known to the skilled person.

The invention also provides methods for modifying phospholipase C enzyme activity-encoding nucleic acids to modify codon usage. In one aspect, the invention provides methods for modifying codons in a nucleic acid encoding a phospholipase to increase or decrease its expression in a host cell. The invention also provides nucleic acids encoding a phospholipase modified to increase its expression in a host cell, phospholipase C enzymes so modified, and methods of making the modified phospholipase C enzymes. The method comprises identifying a “non-preferred” or a “less preferred” codon in phospholipase-encoding nucleic acid and replacing one or more of these non-preferred or less preferred codons with a “preferred codon” encoding the same amino acid as the replaced codon and at least one non-preferred or less preferred codon in the nucleic acid has been replaced by a preferred codon encoding the same amino acid. A preferred codon is a codon over-represented in coding sequences in genes in the host cell and a non-preferred or less preferred codon is a codon under-represented in coding sequences in genes in the host cell.

Alt-PLC has a number of domains. In particular, Alt-PLC has a TIM barrel, a plekstin homology (PH) domain, a VPS9 domain and a RasGEF domain.

As used herein, the term “RasGEF domain” includes an amino acid sequence of about 50-400 amino acid residues in length and having a bit score for the alignment of the sequence to the RasGEF domain profile (SMART HMM) of at least 5. Preferably, a RasGEF domain includes at least about 80-350 amino acids, more preferably about 150-325 amino acid residues, or about 250-320 amino acids and has a bit score for the alignment of the sequence to the RasGEF domain (HMM) of at least 15 or greater. The RasGEF domain (HMM) has been assigned the SMART identifier RasGEF. The RasGEF domain (HMM) has been assigned the PFAM Accession Number PF00617.

As used herein, the term “PH domain” or “Pleckstrin homology domain” includes an amino acid sequence of about 10 to 106 amino acid residues in length and having a bit score for the alignment of the sequence to the PH domain of at least 8. A PH central domain can include at least about 20-80 amino acids, about 40-60 amino acids, or about 15-100 amino acids, and has a bit score of at least 16 or greater. The PH central domain (HMM) has been assigned the PFAM Accession Number PF00169.

As used herein, the term “VPS9 domain” includes an amino acid sequence having a layered fold of six α-helices, with an additional C-terminal helix, and an N-terminal bundle of four α-helices required for soluble expression. The surface residues most conserved within Vps9 domains are found on and around a hydrophobic groove located away from the helix bundle; mutations in several of these conserved residues have been shown to weaken GDP-GTP exchange factor (GEF) activity, suggesting that this is the active site of the Vps9 domain. The VPS9 domain has been assigned the PFAM Accession Number PF02204.

Applicant has generated variants of nucleic acids encoding a phospholipase C enzyme activity, including nucleotides encoding a phospholipase C enzyme in which a domain of the enzyme or a part thereof has been deleted.

Applicant has also demonstrated polypeptides having a phospholipase C enzyme activity having all or part of one or more Alt-PLC domains deleted have an increased phospholipase C enzyme activity relative to polypeptides having a phospholipase C enzyme activity without any domain deletions.

Accordingly, the invention provides variant nucleic acids encoding a phospholipase C enzyme activity. The invention also provides polypeptides having a phospholipase C enzyme activity, wherein the polypeptides have a truncation or deletion of part of all of an Alt-PLC domain.

In one embodiment, the polypeptide having a phospholipase C enzyme activity has a deletion of all or part of the RasGEF domain.

In one embodiment, the polypeptide having a phospholipase C enzyme activity has a deletion of all or part of the PH domain.

In one embodiment, the polypeptide having a phospholipase C enzyme activity has a deletion of all or part of the VPS9 domain.

In one embodiment, the polypeptide having a phospholipase C enzyme activity has a deletion of all or part of the VPS9 domain, and a deletion of all or part of the PH domain.

In one embodiment, the polypeptide having a phospholipase C enzyme activity has a deletion of all or part of the VPS9 domain, a deletion of all or part of the PH domain, and a deletion of all or part of the RasGEF domain.

Host cells for expressing the nucleic acids, expression cassettes and vectors of the invention include bacteria, yeast, fungi, plant cells, insect cells and mammalian cells. Thus, the invention provides methods for optimizing codon usage in all of these cells, codon-altered nucleic acids and polypeptides made by the codon-altered nucleic acids. Exemplary host cells include gram negative bacteria, such as Escherichia coli; gram positive bacteria, such as any Bacillus (e.g., B. cereus) or Streptomyces, Lactobacillus gasseri, Lactococcus lactis, Lactococcus cremoris, Bacillus subtilis. Exemplary host cells also include eukaryotic organisms, e.g., various yeast, such as Saccharomryces sp., including Saccharomyces cerevisiae, Schizosaccharomyces pombe, Pichia pastoris, and Kluyveromyces lactis, Hansenula polyrmorpha, Aspergillus niger, and mammalian cells and cell lines and insect cells and cell lines. Thus, the invention also includes nucleic acids and polypeptides optimized for expression in these organisms and species.

For example, the codons of a nucleic acid encoding a phospholipase C enzyme activity isolated from a bacterial cell are modified such that the nucleic acid is optimally expressed in a bacterial cell, a yeast, a fungi, a plant cell, an insect cell or a mammalian cell. Methods for optimizing codons are well known in the art.

Applicant has purified Alt-PLC enzyme.

In a further aspect of the present invention there is provided an isolated, synthetic or recombinant polypeptide having a phospholipase C enzyme activity, wherein the polypeptide comprises: (a) an amino acid sequence: (i) wherein the polypeptide has a phospholipase C enzyme activity in a Phytophthora; (ii) having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 5 97%, 98%, 99%, or more, or 100% sequence identity to any one of SEQ ID NO: 4, SEQ ID NO: 5, SEQ ID NO: 6, SEQ ID NO: 12, SEQ ID NO: 14, SEQ ID NO: 16, SEQ ID NO: 17, SEQ ID NO: 18, SEQ ID NO: 19, or SEQ ID NO: 30; or (iii) encoded by a nucleic acid as described herein; (b) a Phytophthora polypeptide having a phospholipase C enzyme activity; (c) a polypeptide according to (a) or (b) further comprising a heterologous amino acid sequence or a heterologous moiety; (d) a polypeptide according to (c), wherein the heterologous amino acid sequence or heterologous moiety comprises, or consists of a heterologous (leader) signal sequence, a tag, a detectable label or an epitope; (e) a polypeptide according to any one of (a) to (d), wherein the phospholipase C catalyzes a reaction comprising: PIP2+H2O [symbol] IP3+diacylglycerol and/or PIP2+H2O [symbol] IP3+monoacylglycerol; or (f) the polypeptide according to any one of (a) to (e), wherein: (i) the polypeptide is glycosylated, or the polypeptide comprises at least one glycosylation site, (ii) the polypeptide of (i) wherein the glycosylation is an N-linked glycosylation or an O-linked glycosylation; (iii) the polypeptide of (i) or (ii) wherein the polypeptide is glycosylated after being expressed in a yeast cell; or (iv) the polypeptide of (iii) wherein the yeast cell is a P. pastoris or a S. pombe.

In one embodiment, the Alt-PLC is purified from cell culture.

In one embodiment, the purified Alt-PLC is substantially free of other components. For example, a preparation that contains at least about 90%, 91%, 92%, 93%, 94%, 95%, 96%, 97%, 98% or 99% Alt-PLC.

Applicant has generated Alt-PLC coupled to a tag for purification purposes.

Accordingly, in one aspect the protein may be coupled to a marker, such as a tag used for purification purposes (e.g. 6 His (or HexaHis) tag, Strep tag, HA tag, c-myc tag or glutathione S-transferase (GST) tag). If e.g. a highly purified Alt-PLC protein or variant should be required, double or multiple markers (e.g. combinations of the above markers or tags) may be used. In this case the proteins are purified in two or more separation chromatography steps, in each case utilizing the affinity of a first and then of a second tag. Examples of such double or tandem tags are the GST-His-tag (glutathione-S-transferase fused to a polyhistidine-tag), the 6×His-Strep-tag (6 histidine residues fused to a Strep-tag), the 6×His-tag-100-tag (6 histidine residues fused to a 12-amino-acid protein of mammalian MAP-kinase 2), 8×His-HA-tag (8 histidine residues fused to a haemagglutinin-epitope-tag), His-MBP (His-tag fused to a maltose-binding protein, FLAG-HA-tag (FLAG-tag fused to a hemagglutinin-epitope-tag), and the FLAG-Strep-tag. The marker could be used in order to detect the tagged protein, wherein specific antibodies could be used. Suitable antibodies include anti-HA (such as 12CA5 or 3F10), anti-6 His, anti-c-myc and anti-GST. Furthermore, the Alt-PLC protein could be linked to a marker of a different category, such as a fluorescence marker or a radioactive marker, which allows for the detection of Alt-PLC. In a further embodiment, the Alt-PLC could be part of a fusion protein, wherein the second part could be used for detection, such as a protein component having enzymatic activity.

In one embodiment, the tag is a 10×His tag. In another embodiment, the tag is a 6×His tag.

In one aspect, the isolated, synthetic or recombinant polypeptide can comprise a polypeptide as provided herein that comprises a heterologous signal (peptide) sequence.

In one embodiment, the isolated, synthetic or recombinant polypeptides as provided herein comprise at least one glycosylation site. In one aspect, glycosylation can be an N-linked glycosylation. In one aspect, the polypeptide can be glycosylated after being expressed in a P. pastoris or a S. pombe or in plants, such as oil producing plants e.g. soy bean, canola, rice, sunflower, or genetically-modified (GMO) variants of these plants.

In one aspect, provided herein are isolated, synthetic or recombinant antibodies which specifically bind to a polypeptide as provided herein. In another aspect, the isolated, synthetic or recombinant antibodies are monoclonal or polyclonal antibodies, or are antigen binding fragments thereof. In one aspect, provided herein is a hybridoma comprising an antibody provided herein.

In one embodiment, food supplements for an animal comprise a polypeptide as provided herein, e.g., a polypeptide encoded by the nucleic acid as provided herein. In one aspect, the polypeptide in the food supplement can be glycosylated. In one embodiment, edible enzyme delivery matrices comprise a polypeptide as provided herein, e.g., a polypeptide encoded by the nucleic acid as provided herein. In one aspect, the delivery matrix comprises a pellet. In one aspect, the polypeptide can be glycosylated.

In one embodiment, methods of making an anti-phospholipase C antibody comprise administering to a non-human animal a nucleic acid as provided herein or a polypeptide as provided herein or subsequences thereof in an amount sufficient to generate a humoral immune response, thereby making an anti-phospholipase antibody. Provided herein are methods of making an anti-phospholipase antibody comprising administering to a non-human animal a nucleic acid as provided herein or a polypeptide as provided herein or subsequences thereof in an amount sufficient to generate an immune response.

In a further aspect of the present invention there is provided a protein preparation comprising the polypeptide as described herein, wherein the protein preparation comprises a liquid, a solid or a gel.

Applicant has purified enzymatically active Alt-PLC. Applicant has also demonstrated phospholipase activity in vitro using plant derived phospholipids as a substrate, and phospholipase activity in vitro animal-derived phosphatidylinositols as a substrate.

Accordingly, the polypeptides as provided herein can be used in food processing, brewing, bath additives, alcohol production, peptide synthesis, enantioselectivity, hide preparation in the leather industry, waste management and animal waste degradation, silver recovery in the photographic industry, medical treatment, silk degumming, biofilm degradation, biomass conversion to ethanol, biodefense, antimicrobial agents and disinfectants, personal care and cosmetics, biotech reagents, in increasing starch yield from corn wet milling, and as pharmaceuticals such as digestive aids and anti-inflammatory (antiphlogistic) agents.

In certain embodiments, provided herein are compositions (e.g., phospholipase C enzymes) and methods for producing low phospholipid oils, e.g., oils with a lower phosphatidylinositol content. Any oil, e.g. vegetable oil, e.g. canola oil, soybean oil, or animal oil or fat, e.g., tallow, can be treated with a composition, or by a method, as provided herein. Any foods, edible items, or baking, frying or cooking products can comprise a vegetable oil or animal fat that has been treated with a composition or by a method as provided herein. Vegetable oils modified to be lower phospholipid oils can be used in any foods, edible items or baking or cooking products, e.g., sauces, marinades, condiments, spray oils, margarines, baking oils, mayonnaise, cooking oils, salad oils, spoonable and pourable dressings and the like. In one embodiment, provided herein are oils, such as vegetable oils, e.g., canola oil or soybean oil, and foods or baking or cooking products, including sauces, marinades, condiments, spray oils, margarines, mayonnaise, baking oils, cooking oils, frying oils, salad oils, spoonable and pourable dressings, and the like, wherein the oil or food, baking or cooking product has been modified using an enzyme as provided herein. In one aspect, these vegetable oils, e.g. canola oil, castor oil, coconut oil, coriander oil, corn oil, cottonseed oil, hazelnut oil, hempseed oil, linseed oil, meadowfoam oil, olive oil, palm oil, palm kernel oil, peanut oil, rapeseed oil, rice bran oil, safflower oil, sasanqua oil, soybean oil, sunflower seed oil, tall oil, tsubaki oil, varieties of “natural” oils having altered fatty acid compositions via Genetically Modified Organisms (GMO) or traditional “breeding” such as high oleic, low linolenic, or low saturate oils (high oleic canola oil, low linolenic soybean oil or high stearic sunflower oils), animal fats (tallow, lard, butter fat, and chicken fat), fish oils (candlefish oil, cod-liver oil, orange roughy oil, sardine oil, herring oil, and menhaden oil), or blends of any of the above, and foods or baking, frying or cooking products, comprise oils with a lower saturated fatty acid content, including oils low in palmitic acid, myristic acid, lauric acid, stearic acid, caprylic acid (octanoic acid) etc., processed by using a composition or methods as provided herein.

The invention provides compositions, including enzymes of the invention, and methods, for making biodiesel fuels, including any biofuel, e.g., a biodiesel, comprising alkyl esters made from the transesterification of vegetable oils and/or animal fats.

For example, in alternative aspects, polypeptides of the invention, including the mixture of enzymes or “cocktails” of the invention, are used in processes for a transesterification process reacting an alcohol (like ethanol, propanol, butanol, propanol, methanol) with a triglyceride oil contained in a vegetable oil, animal fat or recycled greases, forming fatty acid alkyl esters—including biodiesel—and glycerin. In one aspect, biodiesel is made from soybean oil or recycled cooking oils. Animal's fats, other vegetable oils, and other recycled oils can also be used (and processed by enzymes, e.g., phospholipases, of the invention) to produce a biodiesel, depending on their costs and availability. In another aspect, blends of all kinds of fats and oils are used to produce a biodiesel fuel of the invention using enzymes of the invention.

The invention provides compositions, including enzymes of the invention, and methods, for processing “yellow grease”, a term initially coined by the rendering industry. Yellow grease that can be processed using the compositions and methods of the invention include grease from frying oils, e.g., from deep fryers or restaurants' grease traps, or from various (e.g., lower-quality) grades of tallow from rendering plants. Thus, the invention also provides oils, grease, frying oils, vegetable oils, waste restaurant greases and processes grades of tallow comprising at least one enzyme of this invention.

Yellow grease processed using compositions of the invention, including enzymes, and methods of the invention, can be used to spray on roads, e.g., for dust control, or for animal feed additives or feeds, or food supplements.

In another aspect, compositions of the invention, including enzymes, and methods of the invention, can be used to process lipids, e.g., greases such as waste restaurant greases to make a biofuel, e.g., a biodiesel fuel, e.g., for cars, buses, trucks or boats. In one aspect, biodiesel made using a composition or method of the invention can be generated from any renewable plant source, e.g., soybeans, and/or from a grease, such as the “yellow grease”. Compositions of the invention, including enzymes, and methods of the invention, can be used to process “SVO”, or “straight vegetable oil”, including any vegetable oil that can fuel a diesel engine, e.g., wherein the processing comprises transesterification of lipids in the fuel, e.g., for use in lower temperatures.

Compositions of the invention, including enzymes, and methods of the invention, can be used to process “WVO”, or waste vegetable oil, to make, e.g., a yellow grease, including the grease from restaurants; in one aspect, the grease has to be filtered to remove food particles.

Yellow grease processed by compositions of the invention, including enzymes, and methods of the invention, can fall in the category of SVO/WVO, including any grease, e.g., a restaurant waste grease, that can contain beef tallow and other animal products.

The invention provides methods for making a biofuel comprising: (A) (a) providing a polypeptide having a phospholipase C enzyme activity according to the present invention, or a phospholipase C enzyme activity encoded by a nucleic acid (polynucleotide) sequence of the present invention, or a polypeptide having a phospholipase C enzyme activity made by a method of the present invention; (b) providing a biomass composition comprising a lipid or an alkyl ester: (c) contacting the phospholipase enzyme of (a) with the biomass composition of (b) to generate a biofuel, or to transesterify the lipid or alkyl ester; (B) the method of (A), wherein the biofuel is or comprises a biodiesel; (C) the method of (A) or (B), wherein the biomass composition comprising a lipid or an alkyl ester is, or comprises, a vegetable oil and/or an animal fat; (D) the method of any of (A) to (C), wherein the biomass composition comprising a lipid or an alkyl ester is, or comprises, an algae, a vegetable oil, a straight vegetable oil, a virgin vegetable oil, a waste vegetable oil, an animal fat, a grease, a tallow, a lard or a yellow grease; or (E) the method of any of (A) to (D), wherein the phospholipase C enzyme activity is, or comprises, an Alt-PLC polypeptide having a sequence as described herein or set forth in any one of SEQ ID NO: 4, SEQ ID NO: 5, SEQ ID NO: 6, SEQ ID NO: 12, SEQ ID NO: 14, SEQ ID NO: 16, SEQ ID NO: 17, SEQ ID NO: 18, SEQ ID NO: 19, or SEQ ID NO: 30; or any combination thereof.

In one embodiment the present invention provides a method for making a biofuel comprising: (a) providing a polypeptide as described herein (b) providing a biomass composition comprising a lipid; and (c) contacting the polypeptide of (a) with the biomass composition of (b) to generate a biofuel, or to transesterify the lipid.

An exemplary reaction for converting oil to biodiesel is called transesterification. The transesterification process reacts an alcohol (like methanol) with the triglyceride oils contained in vegetable oils, animal fats, or recycled greases, forming fatty acid alkyl esters (biodiesel) and glycerin. The reaction requires heat and a strong base catalyst, such as sodium hydroxide or potassium hydroxide.

Biodiesel is a mixture of fatty acid alkyl esters made from vegetable oils, animal fats or recycled greases. Biodiesel can be used as a fuel for vehicles in its pure form, but it is usually used as a petroleum diesel additive to reduce levels of particulates, carbon monoxide, hydrocarbons and air toxics from diesel-powered vehicles.

Hydrolysis includes hydrolysis of a compound, e.g., a biomass, catalyzed using an enzyme of the instant invention.

Congeneration is the simultaneous production of more than one form of energy using a single fuel and facility. In one aspect, biomass cogeneration has more potential growth than biomass generation alone because cogeneration produces both heat and electricity.

In one aspect, the polypeptides of the invention have hydrolase activity, e.g., a phospholipase C enzyme activity, and/or other related enzymatic activity for generating a fuel (e.g. a bioalcohol, e.g., a bioethanol, biomethanol, biobutanol or biopropanol, or biodiesel) from an organic material, e.g., a biomass, such as compositions derived from plants and animals, including any agricultural crop or other renewable feedstock, an agricultural residue or an animal waste, the organic components of municipal and industrial wastes, or construction or demolition wastes or debris, or microorganisms such as algae or yeast.

In one aspect, polypeptides of the invention are used in processes for converting any biomass, e.g., an animal, algae and/or plant biomass including lipid-comprising biomass to a fuel (e.g. a bioalcohol, e.g., a bioethanol, biomethanol, biobutanol or biopropanol, or biodiesel), or otherwise are used in processes for hydrolyzing or digesting biomaterials such that they can be used as a fuel (e.g. a bioalcohol, e.g., a bioethanol, biomethanol, biobutanol or biopropanol, or biodiesel), or for making it easier for the biomass to be processed into a fuel.

Fuels (including bioalcohols such as bioethanols, biomethanols, biobutanols or biopropanols, or biodiesels) made using the polypeptides of the invention, including the mixture of enzymes or “cocktails” of the invention, can be used with fuel oxygenates to improve combustion characteristics. Adding oxygen results in more complete combustion, which reduces carbon monoxide emissions. This is another environmental benefit of replacing petroleum fuels with biofuels (e.g., a fuel of the invention). A biofuel made using the compositions and/or methods of this invention can be blended with gasoline to form an EI 0 blend (about 5% to 10% ethanol and about 90% to 95% gasoline), but it can be used in higher concentrations such as E85 or in its pure form. A biofuel made using the compositions and/or methods of this invention can be blended with petroleum diesel to form a B20 blend (20% biodiesel and 80% petroleum diesel), although other blend levels can be used up to B100 (pure biodiesel).

The invention also provides processes for making biofuels (including bioalcohols such as bioethanols, biomethanols, biobutanols or biopropanols, or biodiesels) from compositions comprising any biomass, e.g., an animal, algae and/or plant biomass including lipid-comprising biomass. The biomass material can be obtained from agricultural crops, as a byproduct of food or feed production.

In one embodiment, the enzymes, including the mixture of enzymes or “cocktails” of the invention, and methods of the invention can be used in conjunction with more “traditional” means of making ethanol, methanol, propanol, butanol, propanol and/or diesel from biomass, e.g., as methods comprising hydrolyzing lipids by subjecting dried any biomass, e.g., an animal, algae and/or plant biomass including lipid-comprising biomass material in a reactor to a catalyst comprised of a dilute solution of a strong acid and a metal salt; this can lower the activation energy, or the temperature, of cellulose hydrolysis to obtain higher sugar yields; see, e.g., U.S. Pat. Nos. 6,660,506 and 6,423,145.

Another exemplary method that incorporated use of enzymes of the invention, including the mixture of enzymes or “cocktails” of the invention, comprises hydrolyzing any biomass, e.g., an animal, algae and/or plant biomass including lipid-comprising biomass.

The invention provides methods for making motor fuel compositions (e.g., for spark ignition motors) based on liquid hydrocarbons blended with a fuel grade alcohol made by using an enzyme or a method of the invention. In one aspect, the fuels made by use of an enzyme of the invention comprise, e.g., coal gas liquid- or natural gas liquid-ethanol blends. In one aspect, a co-solvent is biomass-derived 2-methyltetrahydrofuran (MTHF). See, e.g., U.S. Pat. No. 6,712,866.

In one aspect, methods of the invention for the enzymatic degradation of any biomass, e.g., an animal, algae and/or plant biomass including lipid-comprising biomass, e.g., for production of biofuels (including bioalcohols such as bioethanols, biomethanols, biobutanols orbiopropanols, orbiodiesels) from any organic material, and can also comprise use of ultrasonic treatment of the biomass material; see, e.g., U.S. Pat. No. 6,333,181.

Exemplarty enzymes of the present invention having a phosphoplicase C enzyme activity or use in making a fuel include Alt-PLC1, Alt-PLC2, truncations of Alt-PLC1 and Alt-PLC2, and variants of Alt-PLC, including those described herein.

A phospholipase C enzyme activities of the present invention may utilize (e.g., catalyze hydrolysis of) a variety of phospholipid substrates including phosphatidylcholine (PC), phosphatidylethanolamine (PE), phosphatidylserine (PS), phosphatidylinositol(PI), and/or phosphatidic acid or a combination thereof. In addition, these enzymes can have varying degrees of activity on the lysophospholipid forms of these phospholipids. In various aspects, phospholipase C enzyme activities of the invention may show a preference for phosphatidylcholine and phosphatidylethanolamine as substrates.

In one aspect, phospholipase C enzyme activities of the present invention utilize a variety of phospholipid substrates including phosphatidylcholine, phosphatidylethanolamine, phosphatidylserine, phosphatidylinositol, and phosphatidic acid, or a combination thereof. In addition, these enzymes can have varying degrees of activity on the lysophospholipid forms of these phospholipids. In various aspects, phospholipase C enzyme activities of the invention may show a preference for phosphatidylinositol as a substrate.

Applicant has demonstrated enzymatic activity of Alt-PLC in vitro. For example, Applicant has demonstrated production of diacylglycerol, monoacylglycerol, IP₃ and free fatty acids by Alt-PLC enzyme activities.

Accordingly, in one aspect, the polypeptides as provided herein are used to synthesize products. The polypeptides as provided herein can be used in a variety of pharmaceutical, agricultural and industrial contexts, including the manufacture of cosmetics and nutraceuticals.

In a further aspect of the present invention there is provided a method for producing diacylglycerol comprising: (a) providing a phospholipase C enzyme, wherein the enzyme comprises a polypeptide as described herein, or a polypeptide encoded by a nucleic acid as described herein; (b) providing PIP₂; and (c) exposing the enzyme of step (a) to the PIP₂ of step (b) under conditions that facilitate an enzymatic reaction catalyzed by the phospholipase C enzyme, thereby producing diacylglycerol by a phospholipase C enzyme activity.

In a further aspect of the present invention there is provided a method for producing monoacylglycerol comprising: (a) providing a phospholipase C enzyme, wherein the enzyme comprises a polypeptide as described herein, or a polypeptide encoded by a nucleic acid according as described herein; (b) providing PIP₂; and (c) exposing the enzyme of step (a) to the PIP₂ of step (b) under conditions that facilitate an enzymatic reaction catalyzed by the phospholipase C enzyme, thereby producing monoacylglycerol by a phospholipase C enzyme activity.

In a further aspect of the present invention there is provided a method for producing free fatty acid comprising: (a) providing a phospholipase C enzyme, wherein the enzyme comprises a polypeptide as described herein, or a polypeptide encoded by the nucleic acid as described herein; (b) providing PIP₂; and (c) exposing the enzyme of step (a) to the PIP₂ of step (b) under conditions that facilitate an enzymatic reaction catalyzed by the phospholipase C enzyme, thereby monoacylglycerol and free fatty acid by a phospholipase C enzyme activity.

In a further aspect of the present invention there is provided a method for producing IP₃ comprising: (a) providing a phospholipase enzyme, wherein the enzyme comprises a polypeptide as described herein; or a polypeptide encoded by the nucleic acid as described herein; (b) providing PIP₂; and (c) exposing the enzyme of step (a) to the PIP₂ of step (b) under conditions that facilitate an enzymatic reaction catalyzed by the phospholipase enzyme, thereby producing IP₃ by a phospholipase C enzyme activity.

Accordingly, the enzymes and methods of the invention can be used to achieve a more complete degumming of high phosphorus oils, in particular, rice, soybean, corn, canola, and sunflower oils. For example, in one aspect, upon cleavage by a phospholipase C enzyme activity, phosphatidylinositol is converted to diacylglycerol and phosphoinositol, and because diacylglycerol partitions to the aqueous phase (improving oil yield) and the phosphoinositol partitions to the aqueous phase where it is removed as a component of the heavy phase during centrifugation. An enzyme of the invention, e.g., a phospholipase C enzyme of the invention can be incorporated into either a chemical or physical oil refining process.

The enzymes and methods of the invention can also be used to process and make oils including edible oils, to process and make soaps, used in caustic refining, used in the generation of 1,3-DAG which possesses increased health benefits, making refined oils, making a food including baked goods, oil degumming and vegetable oil processing, preparation of food grade free fatty acid (FFA).

In one aspect, the polypeptides as provided herein can be used in methods of identifying modulators and/or inhibitors of a phospholipase C enzyme activity.

In a further aspect of the present invention there is provided a method for identifying a compound capable of modulating a phospholipase C enzyme activity, the method comprising:

(a) providing a candidate compound; (b) providing (i) a polynucleotide as described herein; or (ii) a polypeptide as described herein; (c) exposing the candidate compound to the polynucleotide or polypeptide; and (d) determining the level of a phospholipase C enzyme activity.

Any of the many phospholipase C activity assays known in the art can be used to examine phospholipase C enzyme activity. Exemplary activity assays include turbidity assays, fluorescent assays, phospholipase assays, thin layer chromatography assays (TLC), cytolytic assays and p-nitrophenylphosphoryl assays.

Protocols for determining PLC enzyme activities are well known in the art.

Thin layer chromatography assays (TLC) to determine phospholipase activity are described, e.g., in Reynolds (1991) Methods in Enzymol. 197:3-13; Taguchi (1975) “Phospholipase from Clostridium novyi type A.I,” Biochim. Biophys. Acta 409:75-85. Thin layer chromatography (TLC) is a widely used technique for detection of phospholipase activity. Various modifications of this method have been used to extract the phospholipids from the aqueous assay mixtures. In some PLC assays the hydrolysis is 5 stopped by addition of chloroform/methanol (2:1) to the reaction mixture. The unreacted starting material and the diacylglycerol are extracted into the organic phase and may be fractionated by TLC, while the head group product remains in the aqueous phase. For more precise measurement of the phospholipid digestion, radiolabeled substrates can be used (see, e.g., Reynolds (1991) Methods in Enzymol. 197:3-13). The ratios of products and reactants can be used to calculate the actual number of moles of substrate hydrolyzed per unit time. If all the components are extracted equally, any losses in the extraction will affect all components equally. Separation of phospholipid digestion products can be achieved by silica gel TLC with chloroform/methanol/water (65:25:4) used as a solvent system (see, e.g., Taguchi (1975) Biochim. Biophys. Acta 409:75-85).

Inositol(1,4,5)P₃ nitrophenol assays may be used to determine phospholipase activity. This assay is based on enzymatic hydrolysis of inositol(1,4,5)P₃ nitrophenol to liberate a yellow chromogenic compound p-nitrophenol, detectable at 405 nm. This substrate is convenient for high-throughput screening.

In one embodiment, a change in a level of a phospholipase C enzyme activity measured in the presence of the candidate compound compared to the activity in the absence of the candidate compound indicate that the test compound modulates a phospholipase C enzyme activity.

In another embodiment, a phospholipase C enzyme activity is measured by providing a phospholipase C substrate and detecting a decrease in the amount of the substrate or an increase in the amount of a reaction product, or, an increase in the amount of the substrate or a decrease in the amount of a reaction product.

A substrate can include PIP₂ (phosphatidylinositol bisphosphate). A reaction product can include IP₃ (inositol triphosphate), diacylglycerol (DAG), monoacylglycerol (MAG) or free fatty acid (FFA).

In a further aspect of the present invention there is provided a method for identifying a compound capable of inhibiting a phospholipase C enzyme activity, the method comprising: (a) providing a candidate compound; (b) providing (i) a polynucleotide as described herein; or (ii) a polypeptide as described herein: (c) exposing the candidate compound to the polynucleotide or polypeptide; and (d) determining the level of inhibition of a phospholipase C enzyme activity.

The term “inhibitor”, as used herein, includes a compound that reduces or inactivates a phospholipase C enzyme activity.

In one embodiment, a decrease in a level of a phospholipase C activity measured in the presence of the candidate compound compared to the activity in the absence of the candidate compound indicates that the test compound inhibits a phospholipase C enzyme activity. In another embodiment, a decrease in the amount of a substrate or an increase in the amount of a reaction product with the candidate compound as compared to the amount of substrate or reaction product without the candidate compound indicates that the test compound is an activator of a phospholipase C enzyme activity. In another embodiment, an increase in the amount of a substrate or a decrease in the amount of a reaction product with the candidate compound as compared to the amount of substrate or reaction product without the candidate compound indicates that the test compound is an inhibitor of a phospholipase C enzyme activity.

In a further aspect of the present invention there is provided a method for determining whether a compound specifically binds to a polypeptide comprising: (a) providing a polypeptide as described herein; (b) providing a candidate compound; (c) exposing the polypeptide to the candidate compound; and (d) determining whether the test compound of step (b) specifically binds to the polypeptide.

As used herein, the term “binding” means the physical or chemical interaction between two proteins or compounds or associated proteins or compounds or combinations thereof, including the interaction between an antibody and a protein. Binding includes ionic, non-ionic, hydrogen bonds, Van der Waals, hydrophobic interactions, etc. The physical interaction, the binding, can be either direct or indirect, indirect being through or due to the effects of another protein or compound. Direct binding refers to interactions that do not take place through or due to the effect of another protein or compound but instead are without other substantial chemical intermediates. Binding may be detected in many different manners. Methods of detecting binding are well-known to those of skill in the art.

Applicant has characterised accumulation of PIP₂, a phospholipase substrate of a phospholipase C activity, when Phytophthora is contacted with a composition comprising an inhibitor.

Accordingly, in a further aspect of the present invention there is provided a method of inhibiting Phytophthora growth comprising contacting Phytophthora with a composition comprising an inhibitor of a Phytophthora phospholipase C enzyme activity.

Inhibition of Phytophthora growth includes reducing the germination of sporangia, zoosporogenesis, zoospore release, encystment, cyst germination, hyphal growth etc.

Accordingly, in a further aspect of the present invention there is provided a method of decreasing Phytophthora viability comprising contacting Phytophthora with a composition comprising an inhibitor of a Phytophthora phospholipase C enzyme activity.

In a further aspect of the present invention there is provided a method of preventing and/or treating infection of plants with Phytophthora comprising contacting a plant with a composition comprising an inhibitor of a Phytophthora phospholipase C enzyme activity.

The term “preventing”, as used herein, includes reducing the probability that a Phytophthora infection will be established in a plant or plants.

The term “treating”, as used herein, includes reducing, stabilizing, or slowing the growth Phytophthora in a plant or plants.

In a further aspect of the present invention there is provided a method of controlling growth of Phytophthora in crops of cultivated plants comprising contacting Phytophthora with a composition comprising an inhibitor of a Phytophthora phospholipase C enzyme activity.

In a further aspect of the present invention there is provided a method of improving Phytophthora-sensitive plant growth comprising contacting the plant with a composition comprising an inhibitor of a Phytophthora phospholipase C enzyme activity.

In a further aspect of the present invention there is provided a composition comprising an inhibitor of a Phytophthora phospholipase C enzyme activity when used for inhibiting Phytophthora growth.

In a further aspect of the present invention there is provided a composition comprising an inhibitor of a Phytophthora phospholipase C enzyme activity when used for preventing Phytophthora growth.

In one embodiment the Phytophthora is selected from the group consisting of Phytophthora infestans, Phytophthora sojae, Phytophthora ramorurn, Phytophthora capsici, Phytophthora phaseoli, Phytophthora nicotiane var. parasitica, Phytophthora palmivora, Phytophthora citrophora, Phytophthora cactorum, Phytophthora syringe, Phytophthora alni, Phytophthora cinnamomi, Phytophthora fragariae, Phytophthora kemoviae, and Phytophthora quercine.

The methods, compounds and compositions of the present invention are suitable for controlling such disease on a number of plants and their propagation material.

In some embodiments, the plant is Pome fruit, and the compound is applied in an amount effective to treat or control Phytophthora crown, collar, root and fruit rot caused by Phytophthora spp.

In some embodiments, the plant is Peppers, and the composition is applied in an amount effective to treat or control a Phytophthora disease selected from the group consisting of: Damping-off and root rot caused by Phytophthora spp. or Phytophthora blight caused by Phytophthora capsici.

In some embodiments, the plant is Tomato, and the composition is applied in an amount effective to treat or control late blight caused by Phytophthora infestans.

In some embodiments, the plant is Soybean, and the composition is applied in an amount effective to treat or control Phytophthora root and stem rot caused by Phytophthora sojae.

In some embodiments, the plant is Grape, and the composition is applied in an amount effective to treat or control Phytophthora crown and root rot caused by Phytophthora spp.

In some embodiments, the plant is Potato, and the composition is applied in an amount effective to treat or control late blight and Pink rot caused by Phytophthora spp.

In some embodiments, the plant is Pineapple, and the composition is applied in an amount effective to treat or control Phytophthora heart rot caused by Phytophthora cinnamomi and Phytophthora parasitica.

The compositions of this invention and useful in the methods of this invention can be formulated in conventional ways. Examples of useful formulations include slurries, solid seed coatings, soaks, dusts on the surface of the seed or tuber, solutions, suspensions, emulsions, wettable powders, emulsifiable concentrates, and the like.

The formulations, in general, comprise about 1% to 99% by weight of active ingredient.

A preferred method of applying a composition of the invention and useful in the methods of this invention, or an agrochemical composition comprising a composition of the invention and useful in the methods of this invention, is foliar application. The frequency of application and the rate of application will depend on the risk of infestation by Phytophthora. However, in some embodiments the compositions can also penetrate the plant through the roots via the soil (systemic action) by drenching the locus of the plant with a liquid formulation, or by applying the compositions in solid form to the soil, e.g. in granular form (soil application). In crops of water such as rice, such granulates can be applied to the flooded rice field. The compositions may also be applied to seeds (coating) by impregnating the seeds or tubers either with a liquid formulation of the fungicide or coating them with a solid formulation.

The compositions can be applied to the crop area or plant to be treated, simultaneously or in succession with further compounds. These further compounds can be e.g. fertilizers or micronutrient donors or other preparations which influence the growth of plants. They can also be selective herbicides as well as insecticides, fungicides, bactericides, nematicides, molluscicides, plant growth regulators, plant activators or mixtures of several of these preparations, if desired together with further carriers, surfactants or application promoting adjuvants customarily employed in the art of formulation.

The compositions can additionally comprise additional additives such as surfactants, solid or liquid diluents, pigments, thickeners, and the like. Suitable carriers and adjuvants can be solid or liquid and are substances useful in formulation technology, e.g. natural or regenerated mineral substances, solvents, dispersants, wetting agents, tackifiers, thickeners, binders or fertilizers.

Furthermore, the compositions can additionally comprise at least one fungicide. In one embodiment the at least one fungicide is selected from the group consisting of: mancozeb, maneb, zineb, thiram, propineb, metiram, copper hydroxide, copper oxychloride, Bordeaux mixture, captan, folpet, amisulbrom, azoxystrobin, trifloxystrobin, picoxystrobin, kresoxim-methyl, fluoxastrobin, pyraclostrobin, famoxadone, fenamidone, metalaxyl, mefenoxam, benalaxyl, cymoxanil, propamocarb, dimethomorph, flumorph, mandipropamid, iprovalicarb, benthiavalicarb-isopropyl, valiphenal, zoxamide, ethaboxam, cyazofamid, fluopicolide, fluazinam, chlorothalonil, dithianon, fosetyl-AI, phosphorous acid, tolylfluanid, and 4-fluorophenyl (IS)-I-({[(IR,S)-(4-cyanophenyl)ethyl]sulfonyl}methyl)propylcarbamate.

In another embodiment the compositions can additionally comprise at least one zoospore attractant. In one embodiment the at least one zoospore attractant is selected from the group consisting of C4-C8 aldehydes or ketones selected from the group consisting of isovaleraldehyde, 2-methylbutyraldehyde, valeraldehyde, isobutyraldehyde, butyraldehyde, 4-methylpentanal, 3,3-dimethylbutyraldehyde, 3-methylthiobutyraldehyde, 2-cyclopropylacetaldehyde, 3-methylcrotonaldehyde, 2-ethylcrotonaldehyde, crotonaldehyde, 2-methylcrotonaldehyde, 3-indolecarbaldehyde, furfural (2-furaldehyde), 2-thiophenecarboxaldehyde, 2-ethylbutyraldehyde, cyclopropanecarboxaldehyde, 2,3-dimethylvaleraldehyde, 2-methylvaleraldehyde, tetrahydrofuran-3-carboxaldehyde, cyclopentanecarboxaldehyde, 3-methyl-2-pentanone, 4,4-dimethyl-2-pentanone, 3,3-dimethyl-2-butanone, and 4-methyl-2-pentanone.

In another embodiment the compositions can additionally comprise at least one substance that induces encystment of zoospores, such as pectin, a metal ion, and an inorganic compound or inorganic salt compound selected from the group consisting of Ca, Zn, Mg, Mn, NaNO₃, KNO₃, and NaCl.

In one aspect the compositions of this invention and useful in the methods of this invention are applied to at least one of the plant, plant foliage, blossoms, stems, fruits, the area adjacent to the plant, soil, seeds, germinating seeds, roots, liquid and solid growth media, and hydroponic growth solutions.

In general, when used against Phytophthora infestans on or in potato plants and tubers, the seed or tuber should be treated with about 50 to about 1200 ppm, preferably between about 300 to about 900 ppm, most preferably, about 700 ppm, per 100 pounds (“cwt”) of seed or tuber, of the composition of this invention or of the compounds useful in the method of this invention.

Plant growth according to the present invention encompasses greater yield, increased quantity of seeds produced, increased percentage of seeds germinated, increased plant size, greater biomass, more and bigger fruit, earlier fruit coloration, and earlier fruit and plant maturation. Growth of a seed may be measured, for instance, in terms of percent germination, time to germination, resistance to seedling diseases or stresses, seedling vigor, or by the stand of a resulting crop.

As a result, the present invention provides significant economic benefit to growers. For example, early germination and early maturation permit crops to be grown in areas where short growing seasons would otherwise preclude their growth in that locale. Increased percentage of seed germination results in improved crop stands and more efficient seed use. Greater yield, increased size, and enhanced biomass production allow greater revenue generation from a given plot of land. It is thus apparent that the present invention constitutes a significant advance in agricultural efficiency.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as broadly described herein.

### EXAMPLES

### Example 1

### Materials and Methods

### Culturing of Phytophthora

Phytophthora cinnamomi strain Du054 was kindly supplied by James Rooks, Deakin University, Waurn Ponds, Australia. Hyphal cultures were grown in broth containing 50 mL clarified V8 juice (Campbell's Soups, Australia), 0.5 g/L peptone and 0.5 g/L CaCl₂. Cultures were maintained on rotation at room temperature (˜23° C.), and broth replenished 12 h prior to experimentation. Plates were made from the above broth with the addition of 15 g of agar. Plate cultures were grown at room temperature; cells were subcultured aseptically by transferring agar plugs to fresh plates when hyphae reached confluence.

### SDS-PAGE and Western Blot Analysis

SDS-PAGE and western blot analyses were performed with 12% separating polyacrylamide gels as described in Sambrook & Russell (2001)(1).

### Immunofluorescence Microscopy

Phytophthora cinnamomi hyphae were grown in V8-peptone alone or with the addition of 0.5 μM of the PLC inhibitor, U-73122. At various time points, a small sample (˜50 mg w/w) was transferred to a sterile tube, and the cells were permeablised in 500 μl methanol at −20° C. for 10 min. The hyphae were then washed in 550 μl tris-buffered saline (TBS) and blocked in 500 μl blocking buffer [1% bovine serum albumin, 1% cold-water fish gelatin, and 0.02% sodium azide in phosphate buffered saline with 1% Tween-20 (PBST)] for 1 h at room temperature. PIP2 was labelled with 500 μl anti-PIP2 FITC antibody (Echelon Biosciences, USA), diluted 1:500 in blocking buffer, and incubated at room temperature for 1 h on rotation in the dark.

Excess anti-PIP2 was removed by 3× washes with 750 μl PBST for 10 min. Hyphae were then spread onto DABCO (Sigma-Aldrich, USA)-coated coverslips.

Images of PIP2-labelled cells were obtained using a confocal microscope (Leica Microsystems TCS SP2, Germany) utilizing 495 nm excitation and 520 nm emission peaks. Images collected in the Z plane through entire hyphae were compiled into a single image; background auto-fluorescence caused by the compilation of stack images was removed by linear brightness reduction.

### Cloning, Expression and Purification of Alt-PLC

The entire Alt-PLC gene was synthesized (Genescript, USA) according to E. coli codon usage, and without intronic regions, and with the introduction of restriction sites for cloning and customized affinity tags for purification before insertion into a pUC57 cloning-vector.

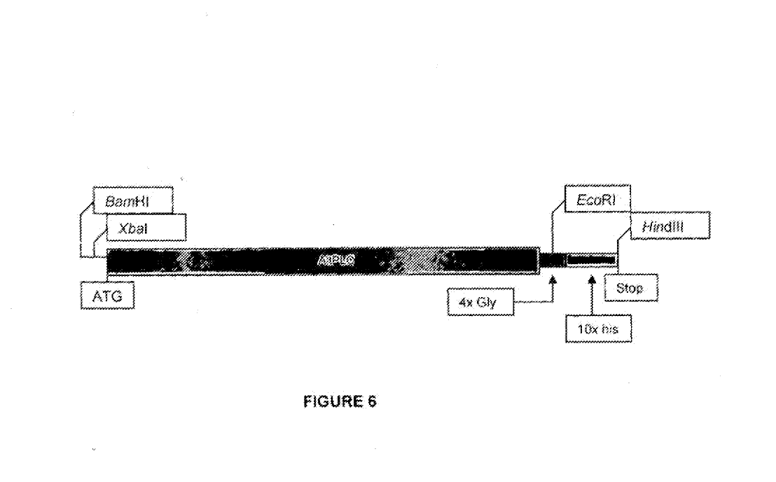

A number of features were included in the synthetic Alt-PLC, including a C-terminal 10× histidine tag linked to the protein by a flexible 4× glycine linker. Four restriction sites were also integrated into the sequence (BamHI, XbaI, EcoRI, and HindIII) without modification of the amino acid sequence. The position of all included features is shown in FIG. 6.

Cloning into pProEx HTb Expression Vector

To produce high yields of Alt-PLC for biochemical analyses, the synthetic Alt-PLC gene was cloned into the pProEX vector system. The gene was ligated into pProEX HTb through the BamHI and HindIII RE sites, producing a double his-tagged protein that useful in preparative-scale isolation procedures.

### Purification of Alt-PLC

One-litre E. coli cultures were grown to an optical density of 0.3-0.5 before induction with 1 mM IPTG and expression continued for 1 h. Cell pellets from batch cultures of 500 ml were resuspended in 10 ml of buffer A3a (5 M guanidine-HCl, 0.5 M NaCl, 20 mM imidazol, 20 mM NaPO4 pH: 7.0) and sonicated for 2 min with intermittent cooling on ice. The lysate was clarified by 2× centrifugation at 13000 rpm for 10 min. Nickel affinity chromatography was performed with a 5 ml histrap HP column on a GE Acta 10 HPLC coupled to a Frac-950 fraction-collector; preparatory scale injections were performed with a 10 ml ‘Superloop’ (GE Healthcare, Switzerland). The program for this purification consisted of binding the target molecule in A3a with a flow rate of 0.2 ml/min followed by a linear elution gradient (0-100%) into buffer B3a (5 M guanidine-HCL, 0.5 M NaCl, 500 mM imidazol, 20 mM NaPO₄ pH: 4.5). Target fractions were collected and pooled over multiple runs. Final purification of the Alt-PLC was performed using denatured size-exclusion chromatography (SEC) with a Superdex 200 Tricorn 10/300 column (GE Healthcare, Switzerland). 5 μl/ml of β-mercaptoethanol was added to the Ni²⁺⁻purified sample and injected in 1 ml batches into the SEC column running an isocratic gradient of A5 (5 M guanidine-HCL 20 mM NaPO₄) at 0.3 ml/min. Target fractions were again pooled, and frozen at −80° C. for future refolding. Where concentration of the protein was required, this sample was dialysed into milliQ H2O and lyophilised.

### In Vitro Refolding of Alt-PLC

Lyophilised Alt-PLC protein was resuspended to the desired concentration in buffer A3a. To this solution, 0.21 g/ml of L-arginine-HCL (Amresco, USA) was added and the solution vortexed until completely dissolved. The sample was then dialysed (using a 10 kDa molecular weight cut-off dialysis tubing, Thermo-Scientific USA) at 4° C. against buffer A4 (500 mM L-arginine-HCL 20 mM NaPO₄ pH: 7.0)—using a ratio of 10 ml sample to 250 ml buffer—without stirring and three quarters of the buffer was replaced every 2 hrs until all guanidine was removed. Monodispersity was checked in each batch using side-scatter from a 600 nm laser passed through the sample; in this test, a polydisperse sample was revealed by ‘sparkling’ within the beam when viewed at 90°.

### Phosphate-Release Assay

Phospholipase activity was determined by measurement of soluble phosphate released by reaction of Alt-PLC with soybean folch fraction ‘asolectin’ (Sigma-Aldrich, USA). Asolectin was suspended in ddH₂O at a concentration of 10 mg/ml by extensive vortexing and pipetting. In each reaction, 400 μL of asolectin solution and 100 μL of purified Alt-PLC (at 0.2 mg/ml or 0.5 mg/ml) was activated by addition of 1 μl of 1 M CaCl₂. After 30 min, the sample was centrifuged at 13000 rpm for 60 min at 4° C.; the supernatant was then transferred to a fresh tube for phosphate quantification—which was determined by a molybdenum colorimetric reaction. Briefly, 10 μl of sample was diluted into 90 μl ddH₂O and, to this, 16 μl of freshly prepared molybdate reagent (6.5 ml of 5 N H₂SO₄, 3.75 ml of 4% ammonium molybdate, 7.5 ml of 0.1 M ascorbic acid, 1.25 ml of 1 mg/l antimony trichloride) was added and mixed by pipetting. The reaction was incubated for 30 min to allow colour development. Absorbance was measured at 750 nm in a nanodrop spectrophotometer (Thermo Scientific, USA).

### Thin-Layer Chromatography (TLC) of Lipids

Lipid samples of 0.5 μl were applied to silica 60 TLC plates and allowed to dry prior to the addition of more sample. Neutral lipids were applied to 10×20 cm pre-absorption zone-bearing silica-60 plates (Sigma-Aldrich, USA) and resolved in a solvent mixture of hexane:diethyl ether:acetic acid (9:1:0.5). Phospholipids were resolved on 20×20 cm silica-60 plates (Merck, Australia), without a pre-absorption zone, in a solvent mixture of chloroform:methanol:acetic acid:water (90:40:12:2). Lipids were detected by spraying the dried TLC plates with a solution of 0.05% rhodamine B (Sigma) in methanol and destaining was performed by repeated spraying with 5 M KOH. Lipid spots were then visualised following UV excitation at 312 nm.

### In-Solution Hydrolysis of PIP₂ by Alt-PLC

Five milligrams of lyophilised, synthetic 1,2-dioctanol-D-myo-phosphatidylinositol(4,5)bisphosphate (diC8-PI(4,5)P2: Echelon Biosciences, USA) was resuspended in 500 μl of 20 mM tris pH:8.0; to this, 100 μl of 1M CaCl₂ and 400 μl of refolded Alt-PLC (0.1 mg/ml) in buffer A4 were added and the reaction allowed to proceed for 60 min. The reaction was stopped by phase separation with the addition of 3.75 ml of chloroform:methanol:12N HCl (2:4:0.1) and mixed thoroughly before the addition of 1.25 ml of chloroform. The solution was then vortexed for 30 sec before addition of 1.25 ml of ddH₂O and then further vortexed for 30 seconds. The sample was then centrifuged at 1000 rpm for 10 min in a swinging bucket rotor at 4° C. The upper aqueous layer (containing inositols) and the lower chloroform layer (containing all lipids and phospholipids) were separately lyophilised in glass tubes and stored at −80° C. for further analysis. Further separation of polar and non-polar lipids, developed by the hydrolysis reaction, was performed by resuspending the lyophilised, chloroform layer in 1 mil 75% methanol and then adding 1 ml of n-hexane. The sample was mixed by vortexing for 30 sec and the phases allowed to separate on ice for 10 min before each was lyophilised in glass tubes and stored at −80° C.

### Mass Spectroscopy of Lipids and Inositols:

All mass spectra were produced using a 6210 MSDTOF mass spectrometer (Agilent Technologies, Australia) with the following conditions: drying gas, nitrogen (7 mL/min, 350° C.); nebulizer gas, nitrogen (16 psi); capillary voltage, ±4.0 kV; vaporizer temperature, 350° C.; and cone voltage, 60V, 5 μL.

### NMR Characterisation of PIP7 Hydrolysis

5 mg of synthetic diC8-PI(4,5)P₂ (Echelon Bioscience, USA) was dissolved in 700 μl CDCl3 with 40 μl 10% DCl in D2O. Initial characterisation (pre-hydrolysis) was performed on a Jeol 400 Mhz (1H) NMR fitted with a cryo-multiprobe. Spectra were acquired at −20° C. Spectra were also recorded on a 800 Mhz (1H) Bruker biospin NMR without a cryo-probe and these samples were measured at 5° C. Due to the higher temperature, the sample was overlayed with an atmosphere of N2 to reduce oxidation. Additional proton spectra were recorded at the beginning and end of characterisation to determine the extent of degradation. Initial characterisation of the synthetic diC8-PI(4,5)P₂ was performed both to assign protons and as a confirmation of the substrate identity. ¹H, 2D ¹H-gTOCSY, ¹H-gTOCSY, gHMBC and gHMQC spectra were recorded providing complete characterisation and confirming diC8-PI(4,5)P₂.

Analyses of monoacylglycerol and free fatty acid fractions were performed in CDCl₃ on a 400 Mhz Jeol NMR with cryo multiprobe at −55° C. All spectra were referenced against tetramethylsilane.

### PIP₂ Accumulation in Phytophthora Cinnamomi

Phosphatidylinositol(4,5)bisphosphate (PI(4,5)P₂) not only plays a critical role in cellular signalling—being a substrate for phospholipase C—but it also provides anchoring sites for structural remodelling proteins such as those of dynamin family (De Matteis & Godi 2004(15)). This specificity of function means that spatial distributions of PIP₂ within the cell are tightly regulated into discrete regions of high and low concentration. This (as opposed to the distribution of more common phospholipids—which is often uniform throughout the plasma membrane) allows the monitoring of PIP₂ distributions, and phenotypic analysis of membrane deformations under PLC agonist and antagonistic conditions.

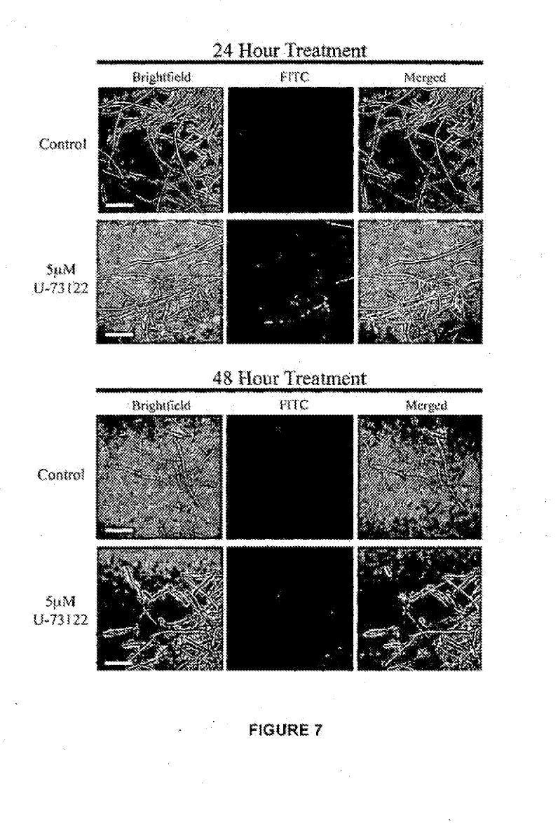

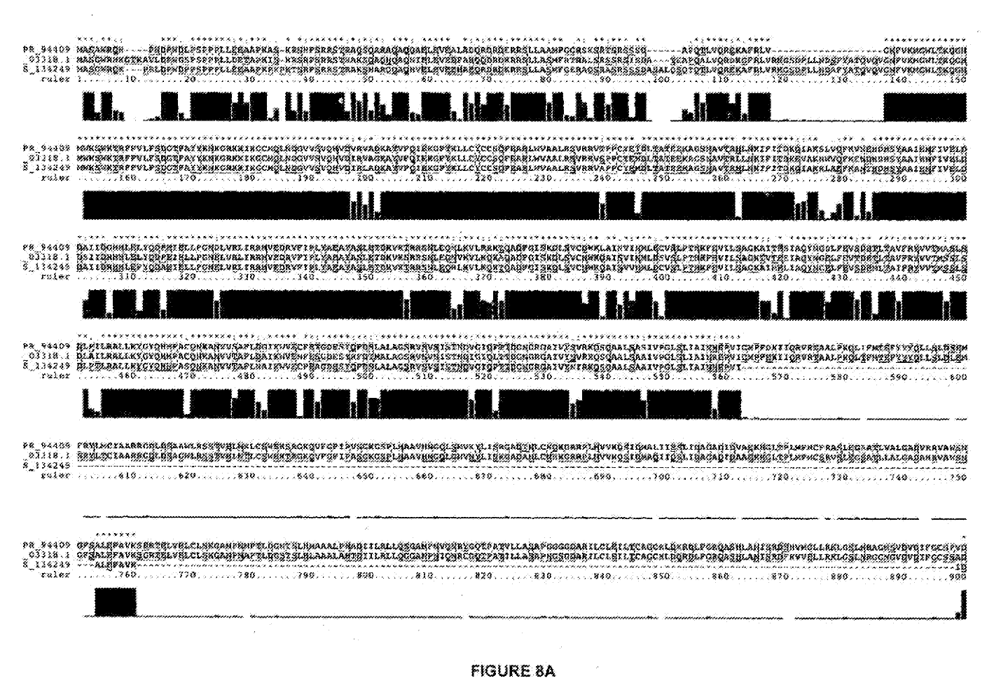

Phytophthora cinnamomi was grown in the presence or absence of the PLC inhibitor, U-73122, and immunofluorescence microscopy was used to compare PIP₂ concentrations in inhibitory and control conditions. FIG. 7 shows that PIP₂ increased when PLC was inhibited by U-73122. This result discounts any non-specific cytotoxic effects of U-73122, and indicates that Phytophthora has a functional, novel phospholipase C.

### Example 2

### Alt-PLC Structure

The amino acid sequence of P. sojae Alt-PLC predicted by automated annotation of the genome is shown in SEQ ID NO: 5. This Alt-PLC is designated ‘Alt-PLC1’.

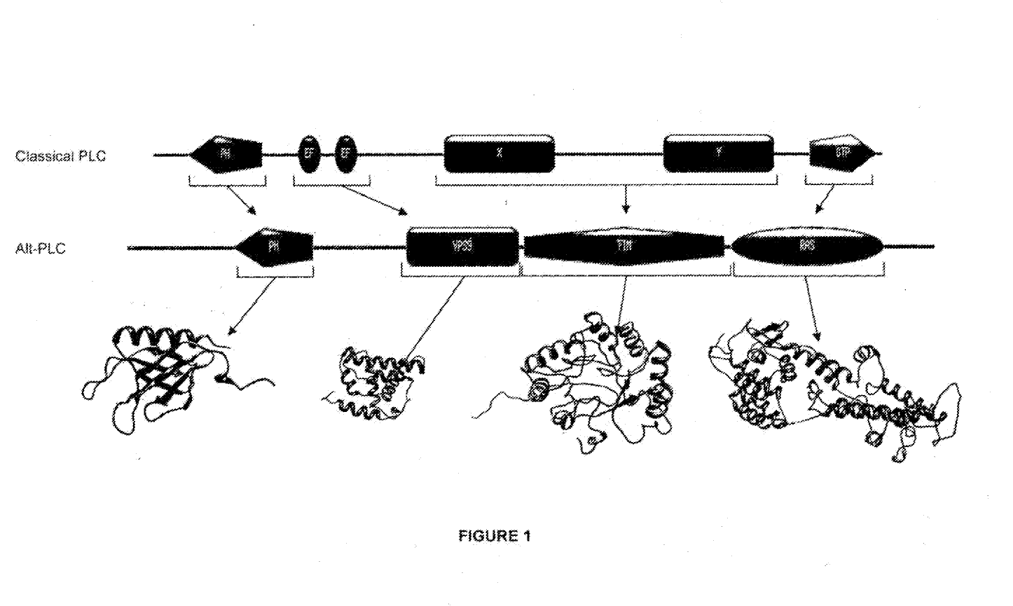

Structure prediction was performed on Alt-PLC by splitting the protein sequence into short sequences of domains and regions between each domain. The Applicant has found Alt-PLC has a pleckstrin homology (PH) domain, a VPS9 domain and a G protein (RAS-GEF)-binding site for PLC regulation. The inter-domain sequence between the VSP9 and RAS domains was subjected to SP4 structural analysis and found to contain a triosephosphate isomerase-like (TIM) barrel-like structure (FIG. 1). This finding was further validated by an alternative structure-prediction algorithm, PHYRE (Kelley et al. 2000 J. Mol. Biol. 299: 499-520). Consistency between two different methods was considered evidence of accurate fold recognition.

Accordingly, Alt-PLC is demonstrated by the Applicant to contain the necessary structural motifs for PI(4,5)P₂ binding and hydrolysis and G-protein regulation (FIG. 1), for example specific binding by PH domains, activation by RAS GTPase, and catalysis by TIM barrels.

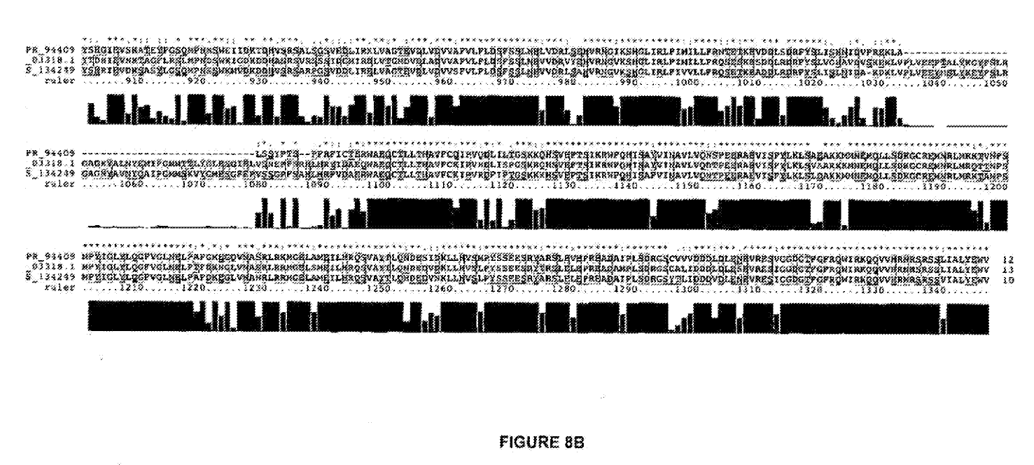

Furthermore the Applicant has identified homologues of the P. sojae Alt-PLC identified in Phytophthora infestans and Phytophthora ranorum (FIG. 8). Homology across such a wide diversity of the Phytophthora genus would suggest that this protein is ubiquitous within, and unique to, the genus Phytophthora.

### Example 3

### Characterisation, Cloning, Codon Optimisation and Expression of Alt-PLC

Alt-PLC was characterised in P. sojae (Ps), and homologs characterised in P. ramorum (Pr) and P. infestans (Pi), but in no other non-Phytophthora organism.

The nucleotide sequence of a mRNA transcript encoding Phytophthora ramorum Alt-PLC1 is shown in SEQ ID NO: 1. The nucleotide sequence of a mRNA transcript encoding Phytophthora sojae Alt-PLC1 is shown in SEQ ID NO: 2. The nucleotide sequence of a mRNA transcript encoding Phytophthora infestans Alt-PLC1 is shown in SEQ ID NO: 3. The amino acid sequence of a Phytophthora ramorum Alt-PLC1 enzyme is shown in SEQ ID NO: 4. The amino acid sequence of a Phytophthora sojae Alt-PLC1 enzyme is shown in SEQ ID NO: 5. The amino acid sequence of a Phytophthora infestans Alt-PLC1 enzyme is shown in SEQ ID NO: 6. The nucleotide sequence of the genomic region encoding a Phytophthora ramorum Alt-PLC1 is shown in SEQ ID NO: 7. The nucleotide sequence of the genomic region encoding a Phytophthora sojae Alt-PLC1 is shown in SEQ ID NO: 8. The nucleotide sequence of the genomic region encoding a Phytophthora infestans Alt-PLC1 is shown in SEQ ID NO: 9. The nucleotide sequence of a codon optimised Phytophthora sojae Alt-PLC1 is shown in SEQ ID NO: 10. The amino acid sequence of a codon optimised Phytophthora sojae Alt-PLC1 polypeptide is shown in SEQ ID NO: 30.

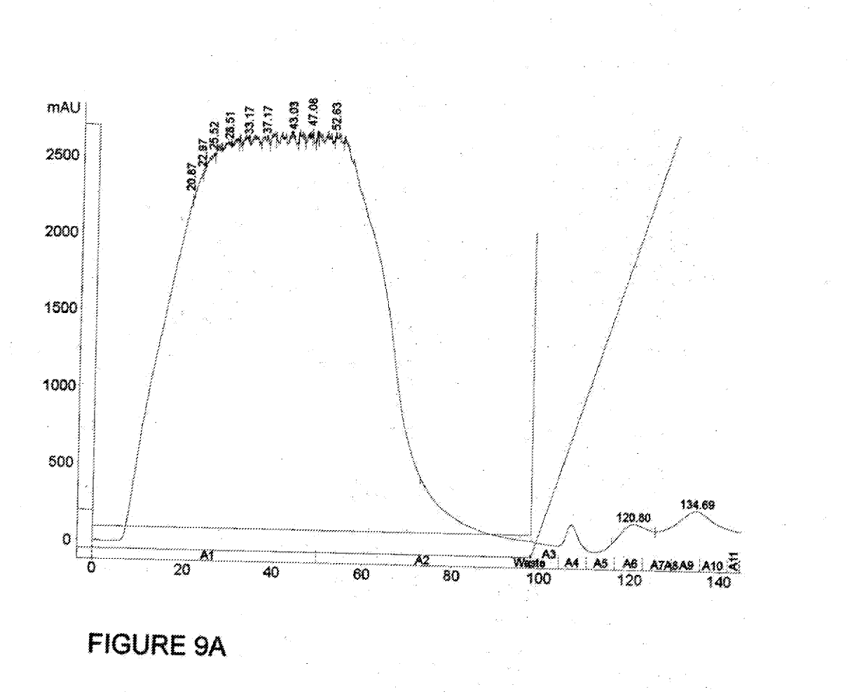

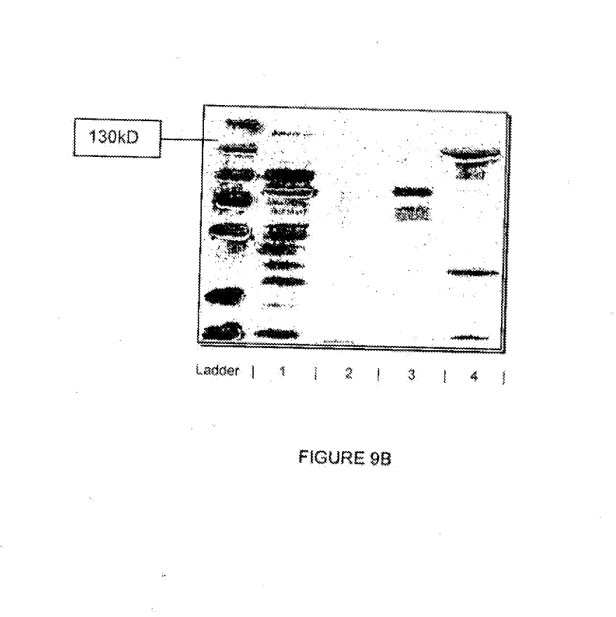

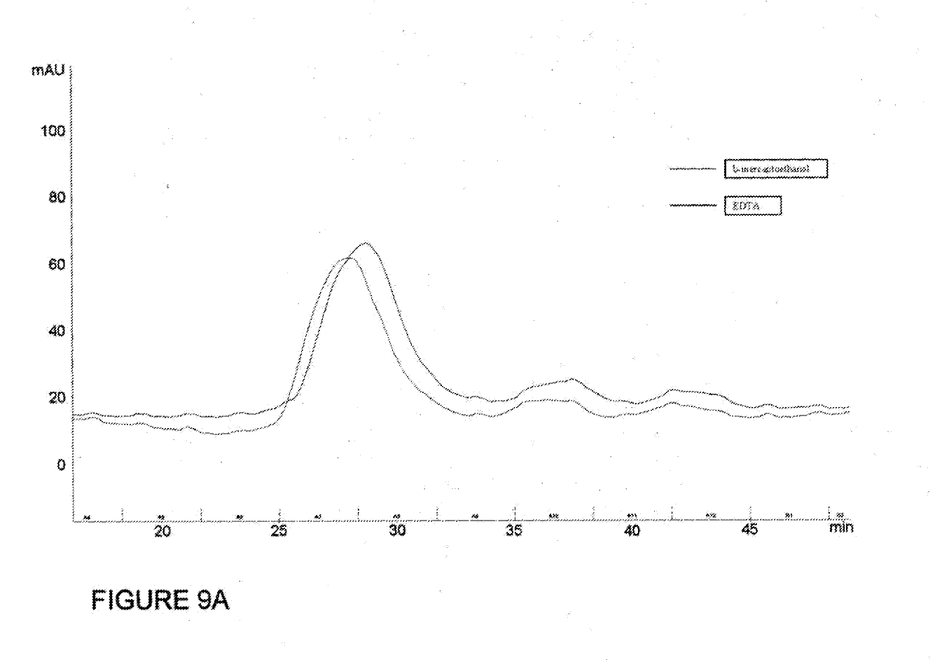

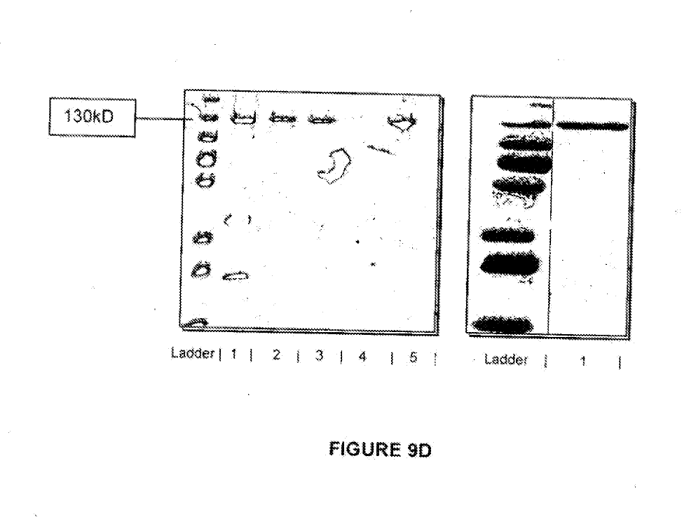

To obtain Alt-PLC protein for enzymatic testing, the coding sequence for P. sojae (Ps) Alt-PLC1 was E. coli codon-optimized and synthesized (by Genescript, USA) before it was inserted into an expression vector and transformed into E. coli. The expressed Alt-PLC protein was purified from bacterial lysates by nickel affinity and size exclusion chromatography (FIG. 9). This protocol yielded isolated Alt-PLC with a purity >99% with an apparent molecular weight of 130 Kda. In vitro refolding parameters for Alt-PLC were identified using L-arginine as the key refolding osmolyte, with refolding performed by dialysis (Example 1).

### Large-Scale Isolation of Alt-PLC

Nickel-affinity and size-exclusion chromatography were performed under denaturing conditions as described in Example 1. Denaturing conditions were required because of an apparent internalisation of the histidine tags. This purification yielded Alt-PLC with two contaminating proteins; thus, additional chromatographic separation was required (FIG. 9).

Size-exclusion (SEC) in the presence of a reducing agent and a metal chelating agent were performed, with both β-mercaptoethanol and EDTA effective in removing contaminants, and β-mercaptoethanol providing slightly greater purity. All subsequent batch purifications were performed under denaturing and reducing conditions.

### Example 4

### Phospholipase Activity of Alt-PLC

An initial characterisation of Alt-PLC phospholipase activity was performed by monitoring release of soluble phosphates from a plant derived phospholipids (asolectin; a mixture of soybean phospholipids) reacted with Alt-PLC. Alt-PLC phospholipase activity was measured in the presence and absence of calcium; the addition of calcium (a required cofactor for PLC) to the reaction was expected to increase the rate of hydrolysis-resulting in higher phosphate levels.

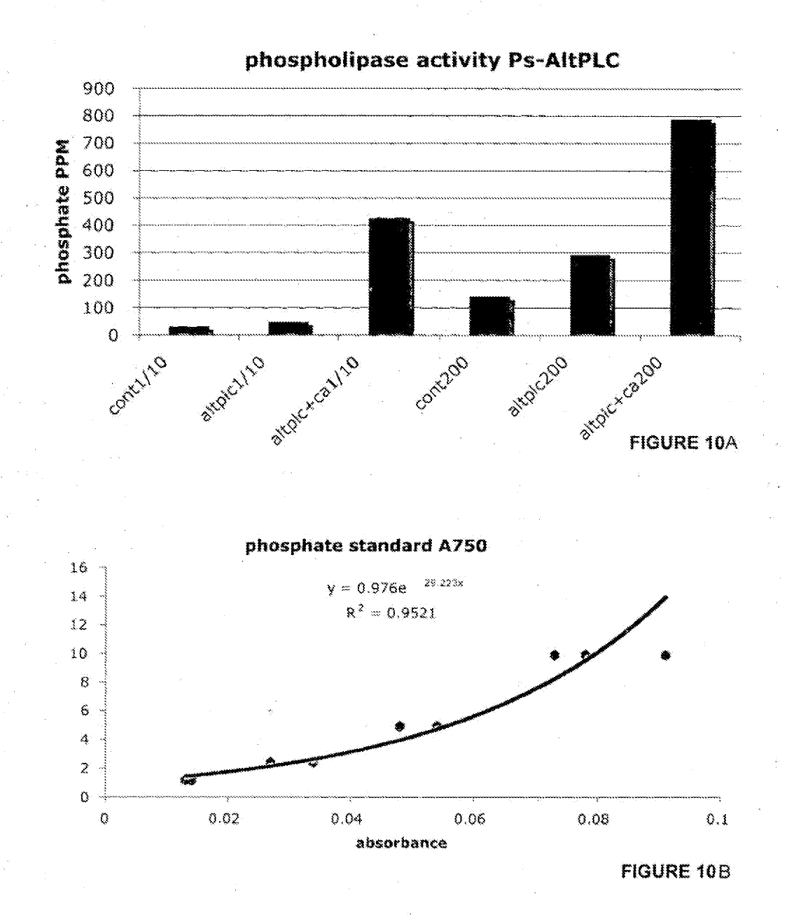

As shown in FIG. 10, a doubling of enzyme concentration was performed with an identical substrate concentration, to provide an internal control and to understand rate maxima. Control samples yielded only minimal phosphate content and this can be attributed to NaPO₄ being present at 20 mM in buffer A4. The Alt-PLC in the absence of added Ca²⁺ reactions show a modest increase in phosphate concentration. Such modest phosphate release is to be expected given the lack of Ca²⁺ cofactor; only modest hydrolysis can occur utilizing trace quantities of Calcium in the milliQ. Finally in the reaction Alt-PLC+Ca²⁺ a ten fold increase in soluble phosphate.

Reactions containing Alt-PLC and Ca²⁺ showed greater than ten fold increase in soluble phosphates compared to the controls, demonstrating phosphate-ester hydrolysis (FIG. 10), and demonstrating that Alt-PLC has a phospholipase enzyme activity, and that Alt-PLC has a phospholipase enzyme activity using plant-derived phospholipids as a substrate.

### Example 5

### Alt-PLC Phospholipase C Activity and IP₃ Production

To test for phospholipase C-like function of Alt-PLC, electrospray ionization mass spectrometry (ESI-MS) was utilized to identify of inositol(1,4,5)triphosphate (IP₃) produced from reactions of Alt-PLC with lipids.

Three separate batches of protein (purified from independent transformations) were prepared and reacted with a mixture of phosphatidylinositols from bovine brain in the presence of CaCl₂ or in the presence of EDTA. Chloroform phase-extraction was performed and the aqueous phase analysed for the presence of IP₃ by ESI-MS.

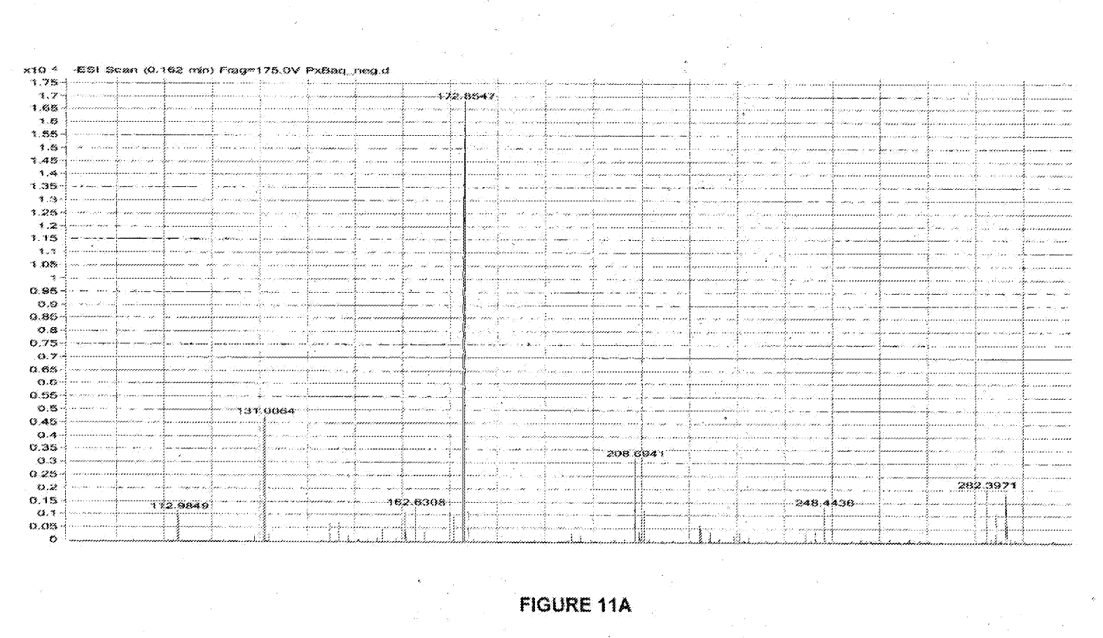

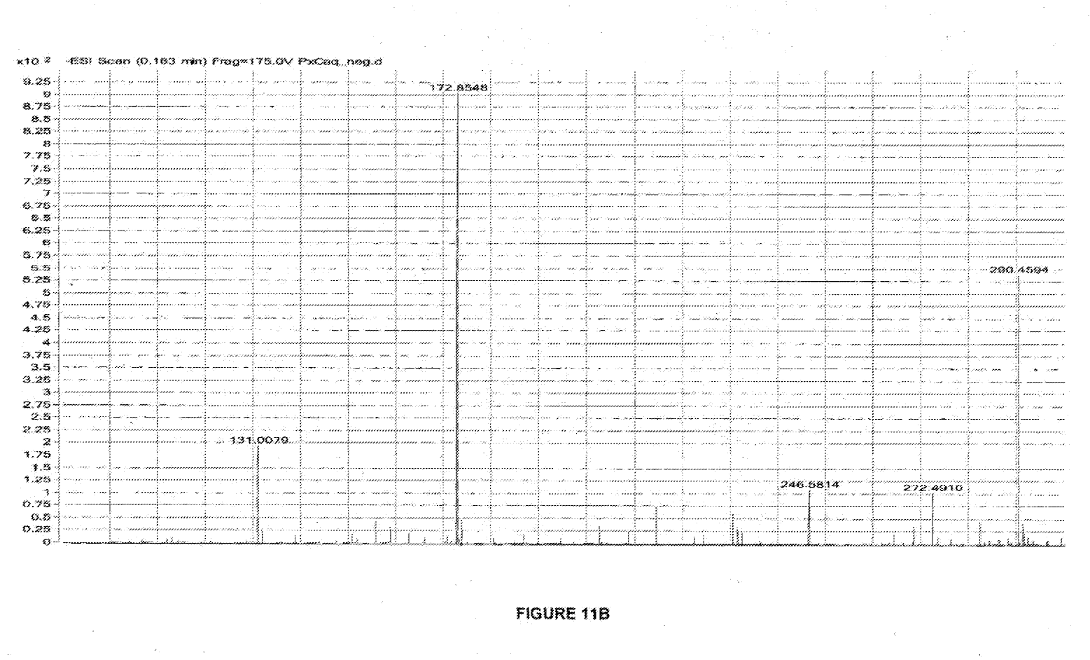

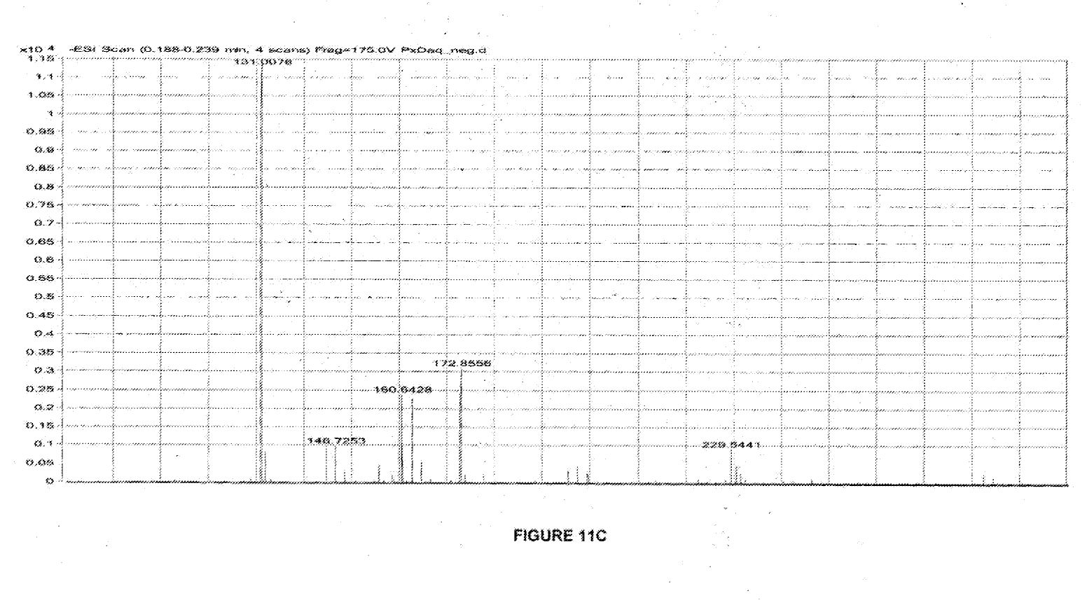

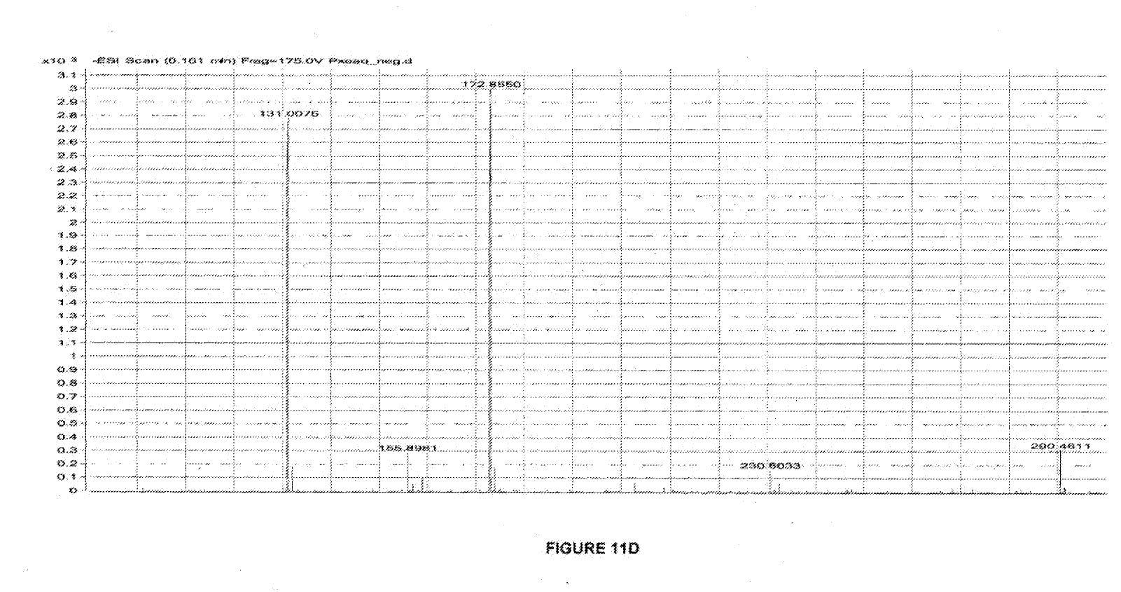

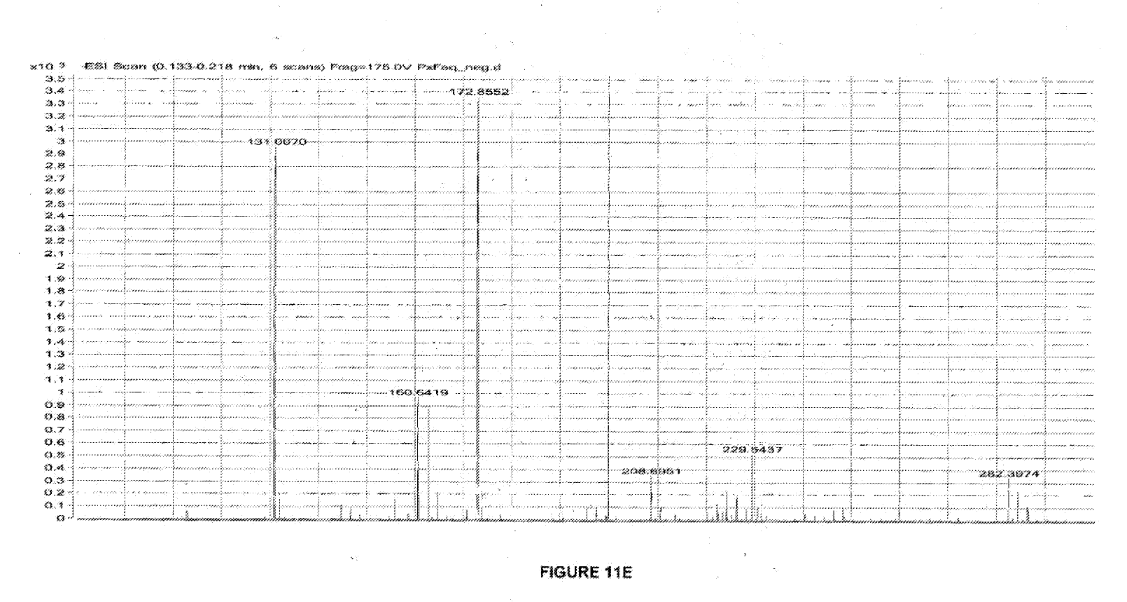

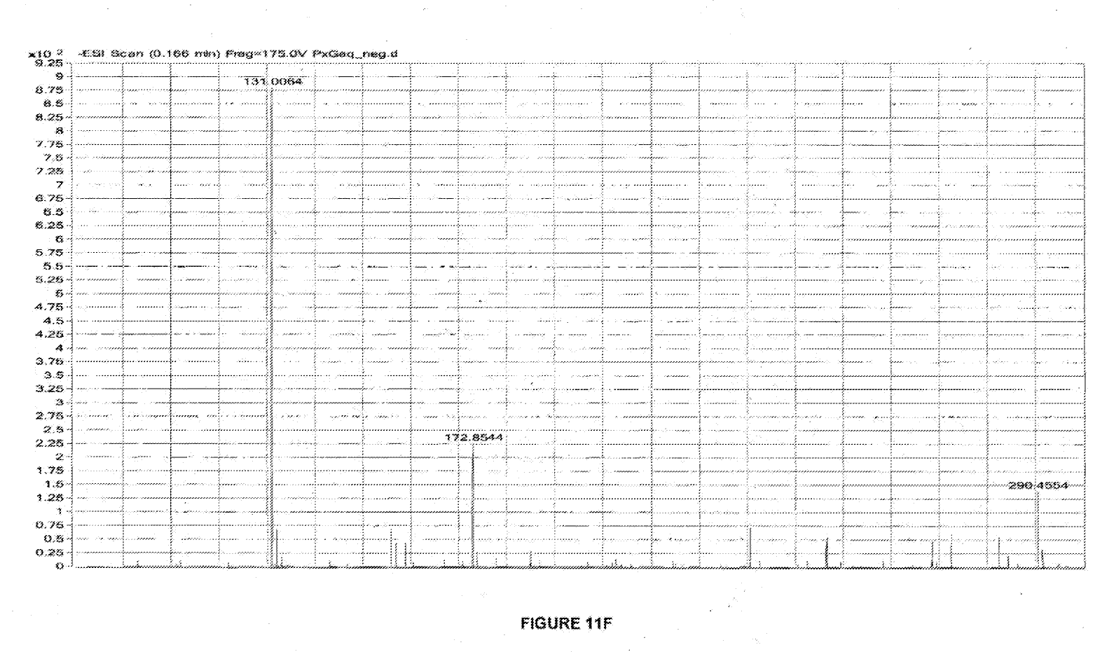

As shown in FIG. 11, the consistent production of peaks at m/z 160.6—the [M-H]⁻ of inositol triphosphate-confirmed the function of Alt-PLC as a phospholipase C.

That IP₃ was produced in the Ca²⁺-activated reactions, but was not produced in the presence of EDTA, demonstrates that Alt-PLC has a phospholipase C enzyme activity, and that Alt-PLC has a phospholipase C enzyme activity when using animal-derived phosphatidylinositols as a substrate.

### Example 6

### Alt-PLC Phospholipase C Activity and Diacylglycerol (DAG) Production

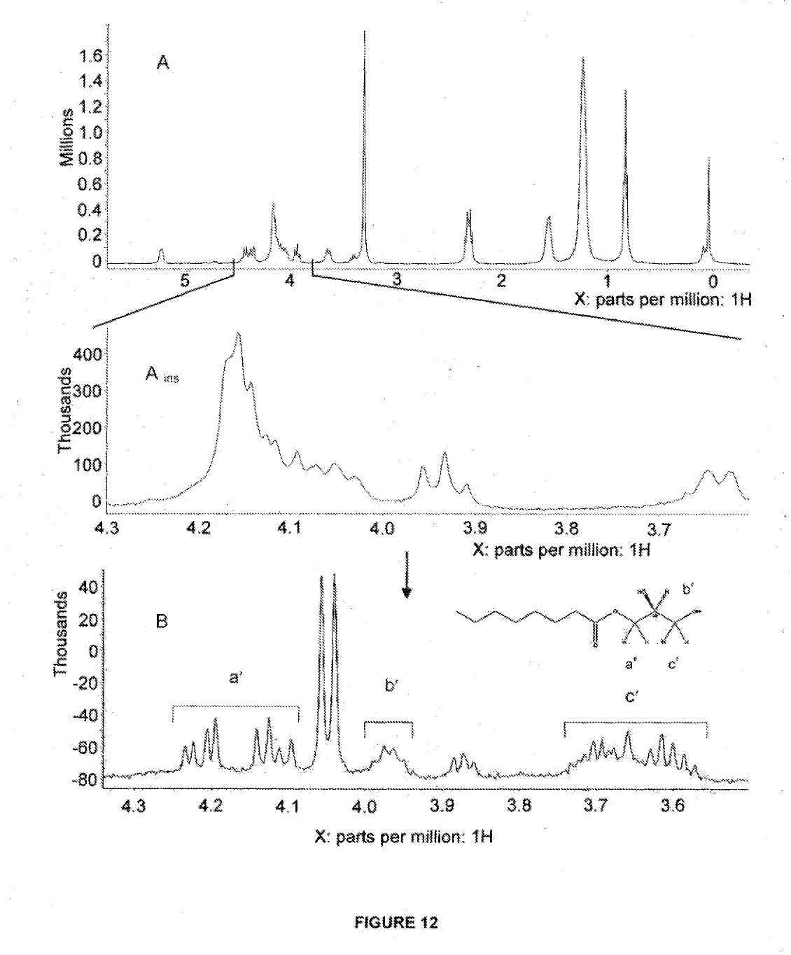

Molecular characterization of Alt-PLC function was conducted by hydrolysis of a 5 mg batch of diC8-PI(4,5)P₂, which was characterized by nuclear magnetic resonance (NMR) spectroscopy prior to use as confirmation of purity (FIG. 12). The reaction was allowed to progress for 1 hr to ensure near complete hydrolysis of the substrate and was stopped by chloroform-HCl/methanol-H₂O phase extraction. The chloroform layer was taken for analysis of lipid species, and the aqueous phase for inositol detection; proteins were removed at the interphase.

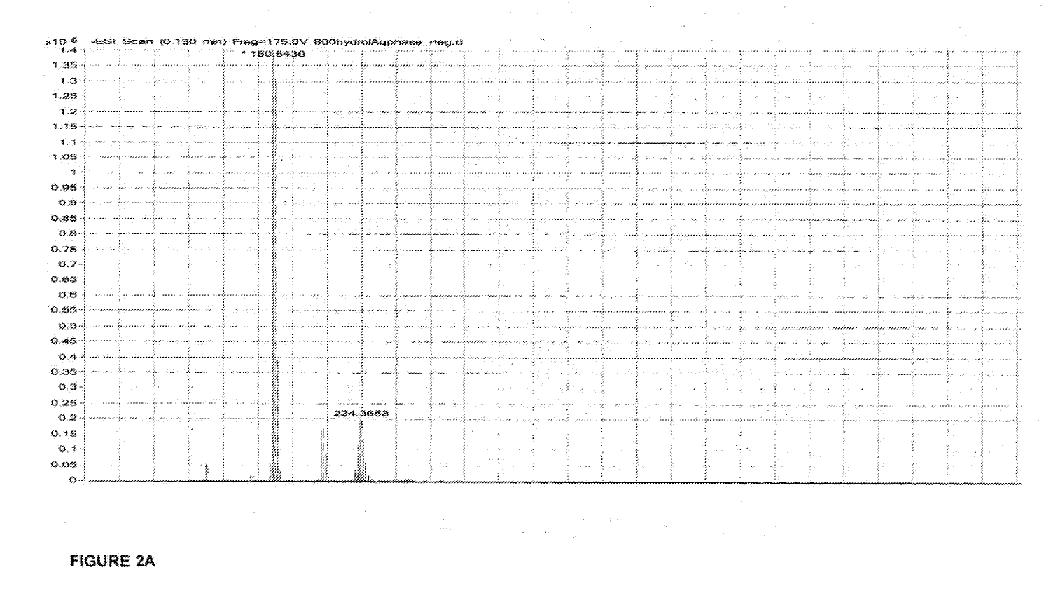

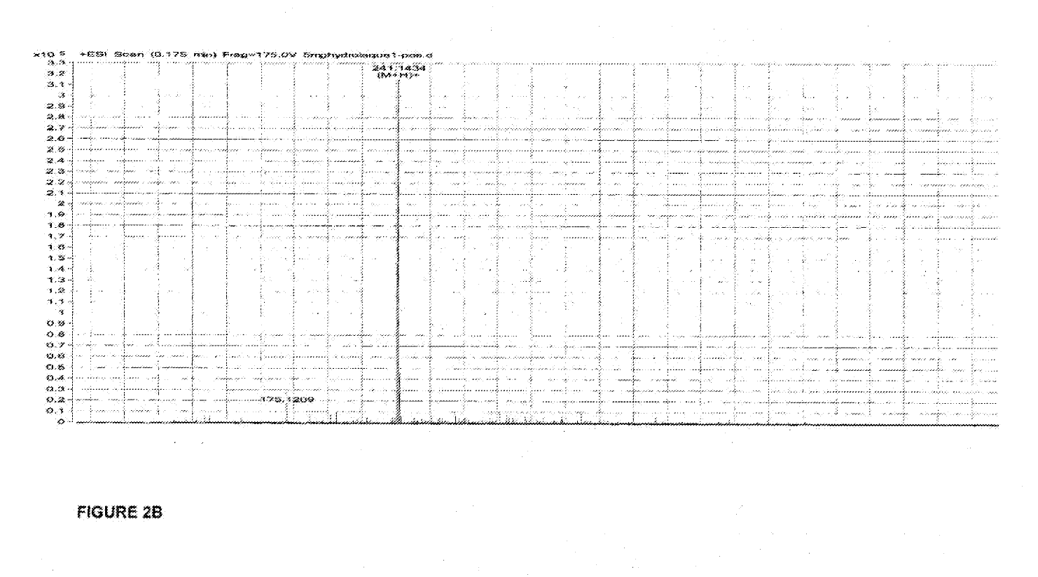

The aqueous phase of the above hydrolysis (chloroform/methanol-H₂O extraction) reaction was analysed by ESI-MS and a strong peak at m/z 160.6 (FIG. 2A) confirmed the presence of IP₃, demonstrating the production of IP₃ by a phospholipase C enzyme activity, consistent with the production of IP₃ by a phospholipase C enzyme activity demonstrated in Example 4.

Following solvent extraction of hydrolysed lipid products, analysis was performed by thin layer chromatography (TLC). This result clearly showed the emergence of a neutral lipid species having a similar Rf value to the DAG control. To further characterize this product, a thin-layer protocol selective for acylglycerols (Kopka et al. 1998. Plant Physiol. 116: 239-250) was applied.

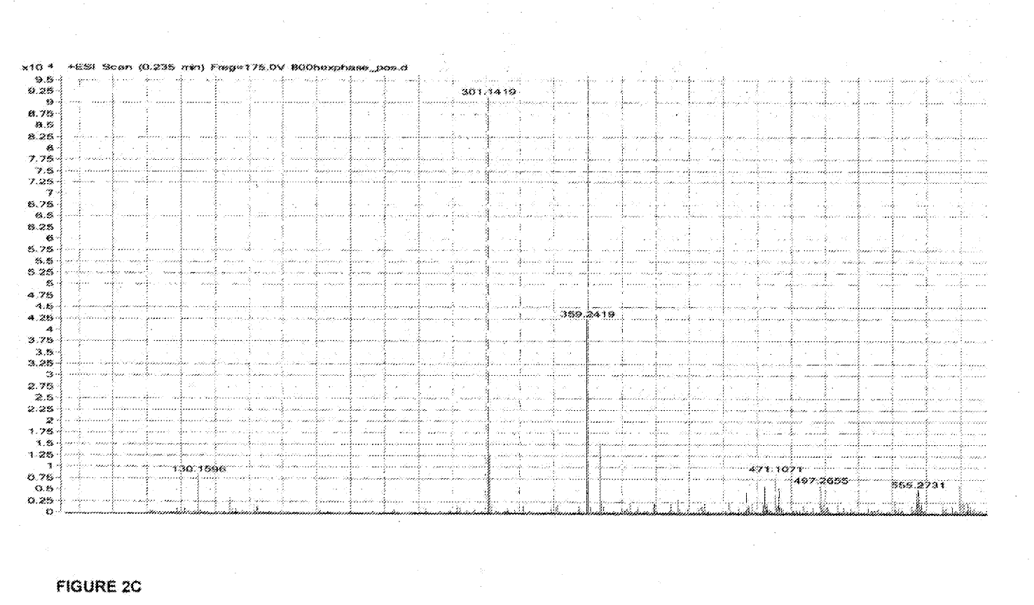

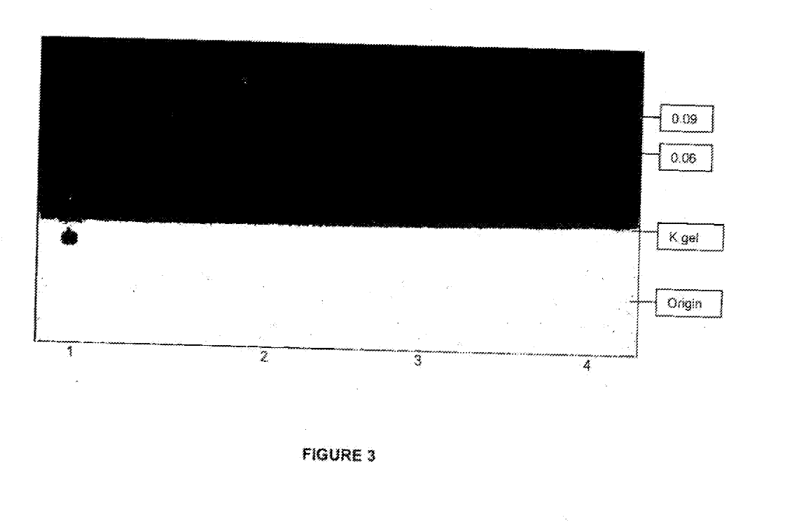

As shown in FIG. 3, the hydrolysis product yielded a band of identical Rf value (0.09) to the DAG control, demonstrating the production of DAG by a phospholipase C enzyme activity.

### Example 7

### Alt-PLC Phospholipase Activity and Monoacylglycerol (MAG) Production

Surprisingly, acylglycerol TLC analysis also identified a second, neutral lipid species (Rf=0.06). Applicant hypothesized that this unknown species may be monoacylglycerol.

To investigate possible monoacylglycerol production, mass spectroscopy (MS) was performed. Prior to MS analysis, lipid hydrolysis products were further fractionated by hexane/methanol-H₂O phase extraction in order to isolate DAG. These fractions were then analysed using positive ion mode mass spectrometry where the [M+Na]⁻ adducts were observed confirming that MAG was being produced by this reaction, demonstrating the production of MAG by a phospholipase C enzyme activity.

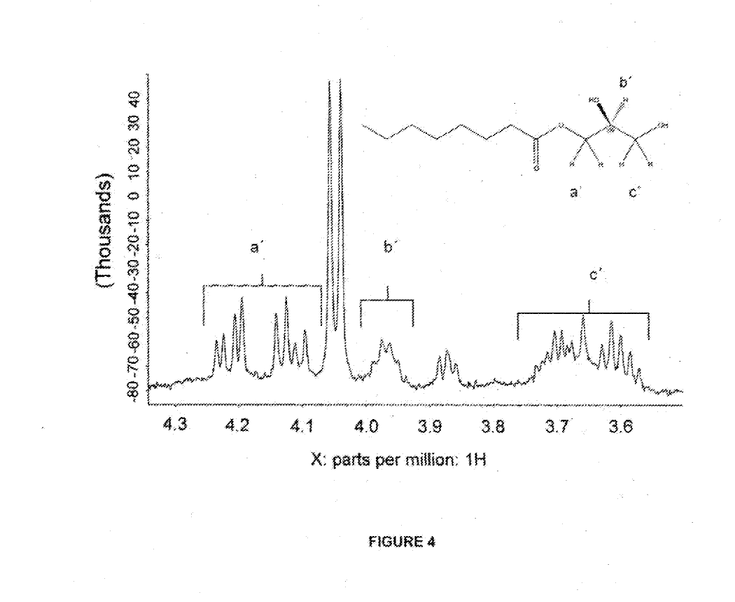

To confirm the production of monoacylglycerol indicated by MS and TLC of neutral lipids generated by Alt-PLC (FIG. 4), ¹H-NMR was performed on the sample. FIG. 12 shows the NMR spectra prior to and following hydrolysis, demonstrating the production of MAG by a phospholipase C enzyme activity.

### Example 8

### Alt-PLC Phospholipase Activity, Monoacylglycerol (MAG) Production, and Free Fatty Acid (FFA) Production

The production of MAG from PIP₂ would also be expected to yield a free fatty acid (FFA); therefore Applicant performed the analysis of the hexane phase by ESI-MS. The free fatty acid was not observed in the negative ion mode. The predominant positive ion peak in the hexane extract can be seen at m/z 301, consistent with the production of a free fatty acid which has covalently bonded to the amine of arginine which was in high concentrations in the hydrolysis buffer, demonstrating the production of FFA by a phospholipase C enzyme activity. Without wishing to be bound by theory, acylated arginine formation indicates that acyl release was a function of enzymatic activity in in vitro conditions rather than any acid hydrolysis occurring in the extraction, since were acid hydrolysis to occur, the radical would have become an aldehyde—which would have been indicated by a mass spectral peak at m/z value 151.2. In addition, NMR analysis of this fraction showed no aldehyde chemical shifts.

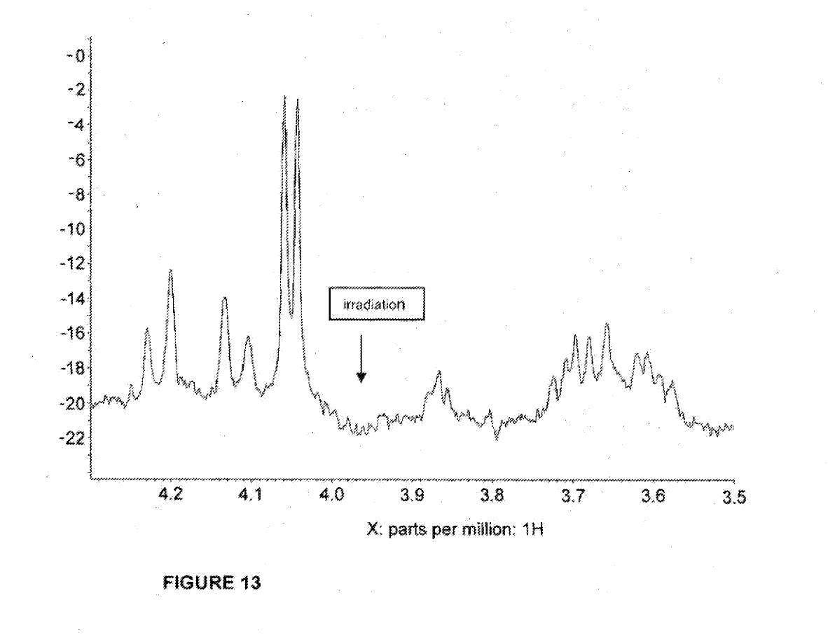

The unusual nature of this double hydrolysis led the Applicant to perform NMR on the MAG and acyl arg fractions as an additional confirmation of the ESI-MS results. The aqueous phase, containing the putative MAG, was subjected to additional chloroform/H₂O phase extraction before lyophilisation and resuspension in CDCl₃. NMR spectra were recorded at 400 MHz at −55° C. FIG. 12 shows the H¹ spectra of the MAG sample compared against the H¹ spectra of PIP₂ prior to hydrolysis. The ¹H-NMR spectrum shown in FIG. 12 identified an end product of Alt-PLC hydrolysis to be 1-monoacylglycerol. The resonances of a′, b′, and c′ are assigned to 1-monoacylglycerol. This spectrum is identical to that previously reported (Compton et al. 2007 J Amer Oil Soc. 84: 343-348). Connectivity of these three resonances was confirmed by homonuclear decoupling of b′. That, as expected, caused a′ and c′ to collapse (FIG. 13).

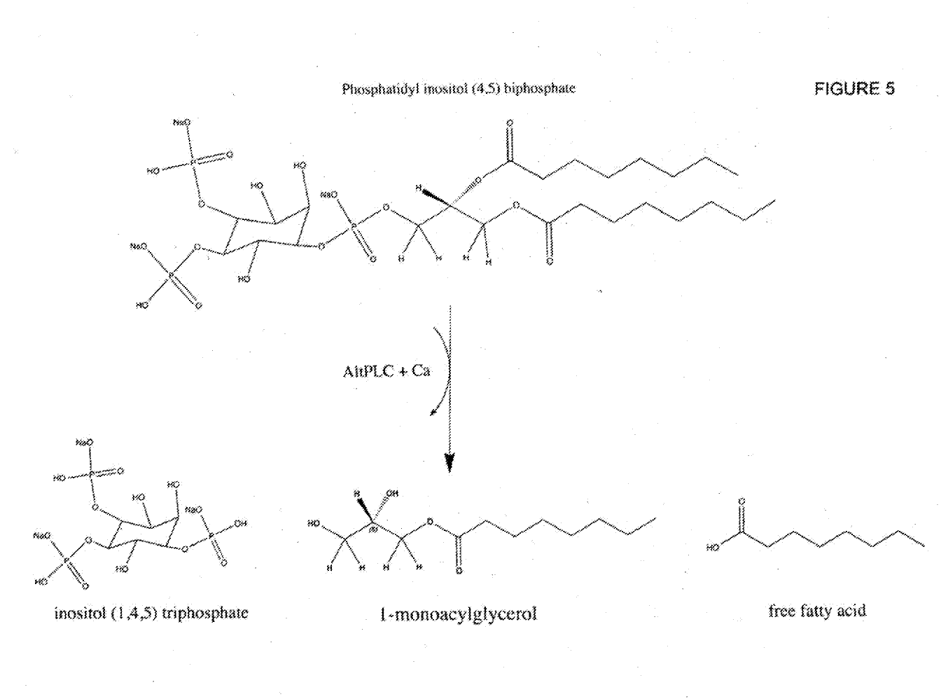

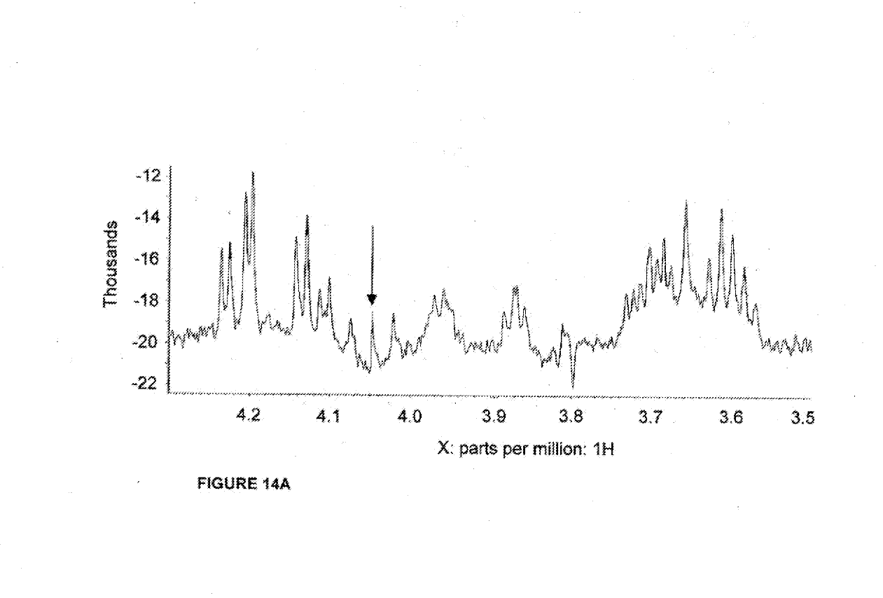

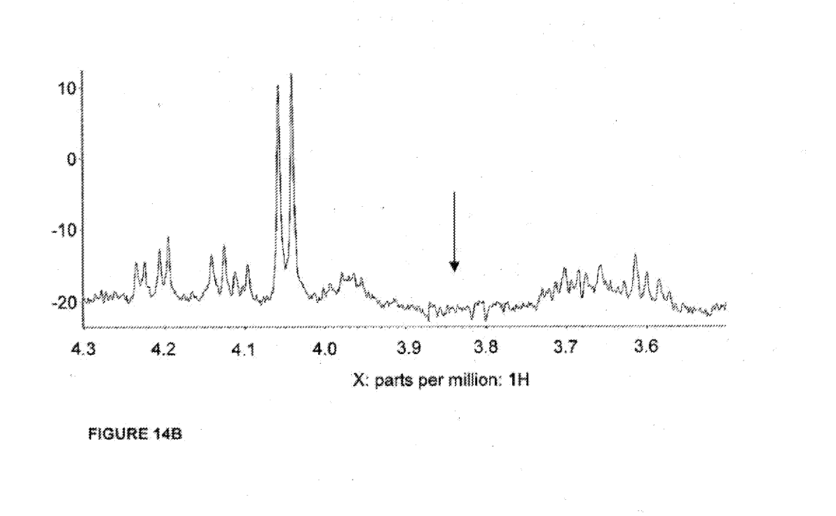

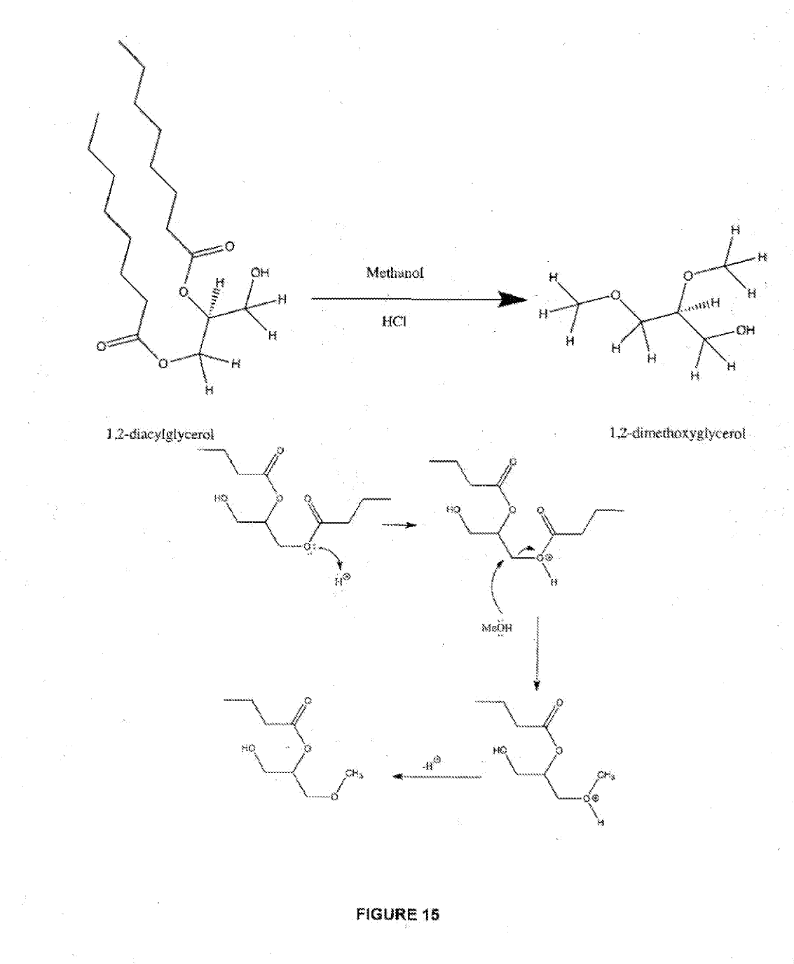

While the apparent doublet at 4.07 ppm in the ¹H-NMR spectra could be mistaken for 2-MAG, or even 1,3-diacylglycerol, following irradiation of the doublet (FIG. 14A) no multiplet collapse occurred at higher or lower frequencies. This result suggested that the ‘doublet’ was in fact two singlets and, given the chemical shift, were most likely methoxys in the same molecule in slightly different chemical environments. Upon examining the common lipid isolation procedure, it was recognised that 1,2-diacylglycerol could have had acyl chain exchange occur in the presence of methanol and acid, yielding 1,2-dimethoxyglycerol—the proposed chemical transformation for which is shown in FIG. 15. Without wishing to be bound by theory. Applicant believes that the remaining protons of this molecule are represented by a resonance at 3.87 ppm and the remaining resonance is not visible—occurring at 3.65 ppm and thus obscured by 1-MAG resonances. Other irradiations were performed looking for connectivity: the triplet (or quintet) at 3.87 was irradiated (FIG. 14B) but showed no J₃ coupling to the doublet; such an effect would be expected if the resonances are a result of 1,2-dimethoxyglycerol as the couplings would be separated by four bonds. Thus, Applicant concludes that the doublet is a methoxy contaminant and the products of Alt-PLC hydrolysis include 1-monacylglycerol, a free fatty acid (FFA) and inositol(1,4,5)triphosphate (FIG. 5).

### Example 9

### PIP₂ Accumulation in Phytophthora Treated with the Inhibitor U-73122 Indicates Inhibition of a Phospholipase C Activity in Treated Cells

Applicant has demonstrated the inhibitor U-73122 markedly reduces hyphal growth rate in Phytophthora (e.g. P. cinnamomi and P. sojae (data not shown)). To examine whether U-73122 inhibits a phospholipase C enzyme activity of Alt-PLC, Applicant compared PIP₂ levels in U-73122 treated Phytophthora to untreated Phytophthora. Applicant observed PIP₂ accumulation in inhibitor treated Phytophthora (FIG. 7), indicating inhibition of a phospholipase C enzyme activity.

### Example 10

### High Throughput Screening of Alt-PLC Activity Using Inositol(1,4,5)P₃ Nitrophenol

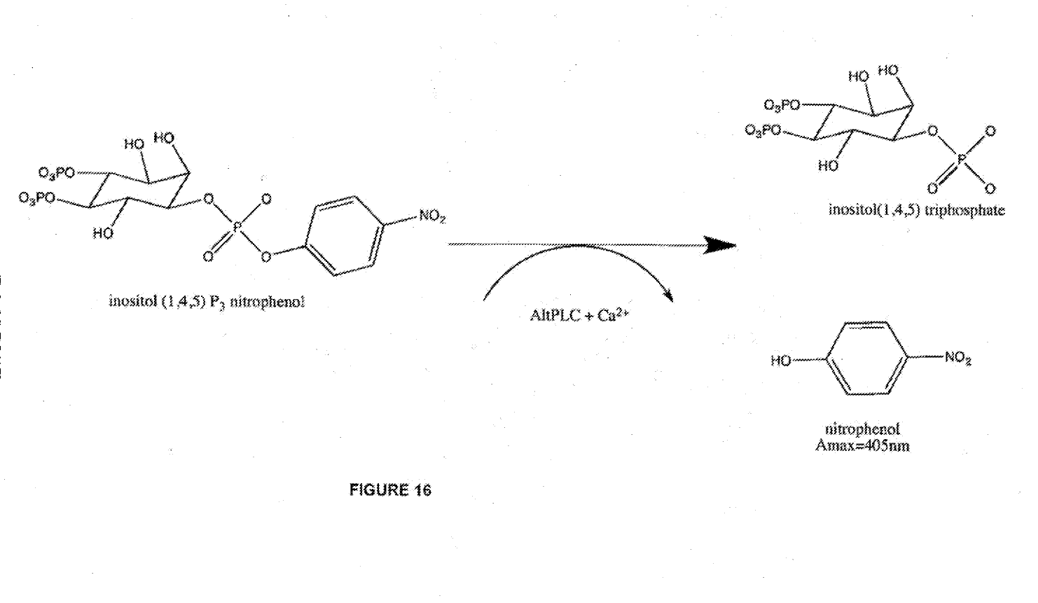

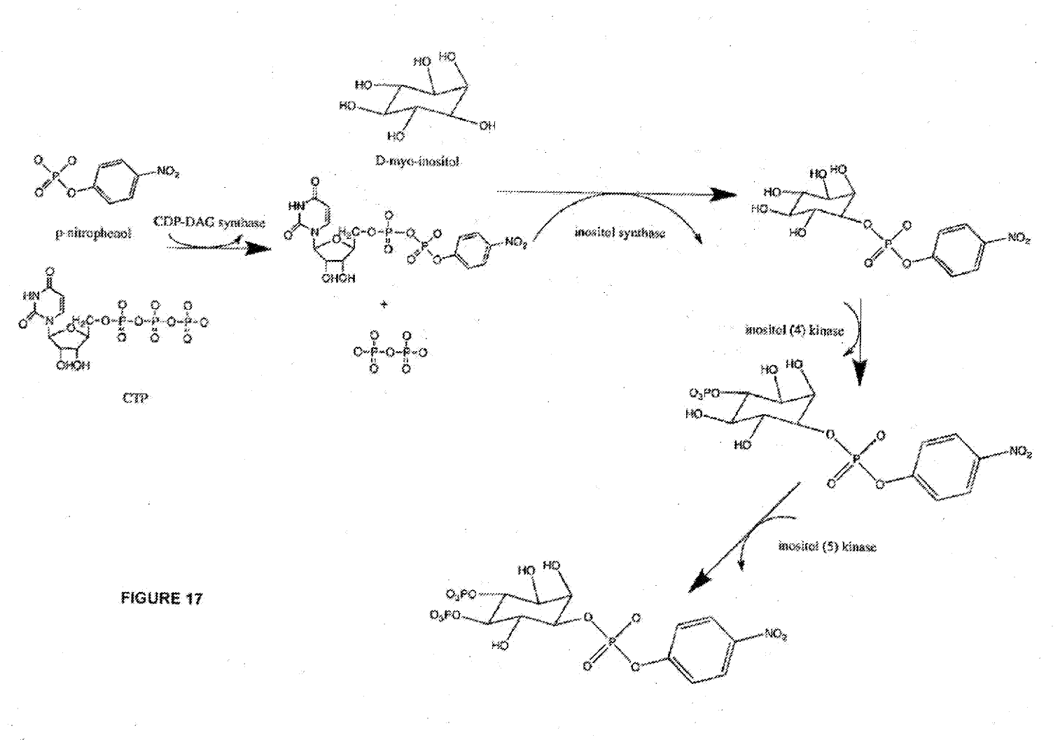

To identify a compound capable of inhibiting a phospholipase C enzyme activity, inositol (1,4,5)P₃ nitrophenol is used to monitor Alt-PLC activity in vitro using a fluorescence microplate reader. In this assay, PLC activity is monitored by examining the generation of nitrophenol which is observable spectrophotometrically with an Amax of 405 nm. FIG. 16 shows a scheme of Alt-PLC catalytic function on inositol(1,4,5)P₃ nitrophenol in the presence of calcium. This reaction yields inositol(1,4,5)triphosphate and nitrophenol (Amax=405 nm) as the end products. FIG. 17 shows a chemical-transformation scheme for the production of inositol(1,4,5)P₃ nitrophenol.

For the assay of Alt-PLC enzyme activity, inositol(1,4,5)P₃ nitrophenol is dissolved in H₂O (total vol, 0.123 mL) giving an optically clear solution, and the pHis adjusted to approximately 5 with solid NaHCO₃. Complete hydrolysis of a small aliquot in 0.01 N NaOH to a total concentration of 70.2 mM p-nitrophenoxide ion (pNP), based on the molar absorptivity at 399 nm (ε, 18, 100 M⁻¹ cm⁻¹) of pNP. A second aliquot is added to the assay buffer and absorbance at 399 nm examined to determine the concentration of pNP. Enzyme activities are measured using inositol(1,4,5)P₃ nitrophenol and varying concentrations of Alt-PLC. The reaction rate is determined.

### Example 11

### Determining Connectivity and Size of Exons in Alt-PLC by Reverse Transcriptase PCR

FIG. 18 shows an intron exon map of P. sojae Alt-PLC from automated genome annotation and oligonucleotide primers used in determining intron/exon structure. RNA from differentiating P. sojae UQ310 cells was isolated and cDNA prepared using standard techniques and PCR reactions were conducted to cross introns 2-9 in three overlapping fragments. Oligonucleotide primer pairs used in the three reactions were: primer pair A=F1880-R2940, primer pair B=F2920-R4120, and primer pair C=F4080-R4560. Gradient PCR reactions on all primer pairs were performed using genomic DNA to determine the optimal annealing temperatures, the annealing temperatures was as follows A=52° C., B=57° C., C=57° C.

### Table 1: Table of Primers Used to Re-Sequence and Annotate Ps-Alt-PLC Transcript

F910 (SEQ ID NO: 20) CGATCATTGATCGTCACC
F1880 (SEQ ID NO: 21) AATACGCTATGCTCGTGGG
F2920 (SEQ ID NO: 22) GCTTGAACGAAGTAGTGGATCGC
R2150 (SEQ ID NO: 23) GAGCACATGAACATTAACGG
R2940 (SEQ ID N: 24) TAAGCCTTTCGGACGTG
R4120 (SEQ ID NO: 25) TATGGCATGCTCGGATTAGC
R4560 (SEQ ID NO: 26) CGACGTCAACATCGTCATCG
R4708 (SEQ ID NO: 27) CACCCACTCGTACAGAGCT
EAG (SEQ ID NO: 28) GCTATCAGTTTGATAGCCT
AVA (SEQ ID NO: 29) GCAGATACAGGCCAATAT

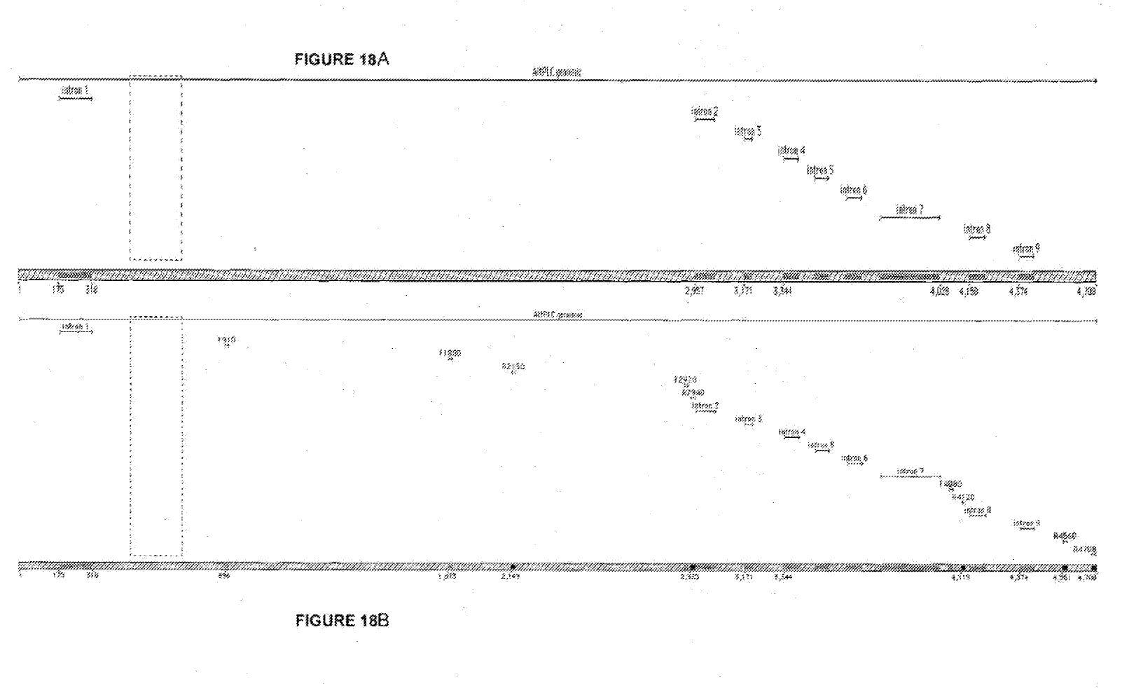

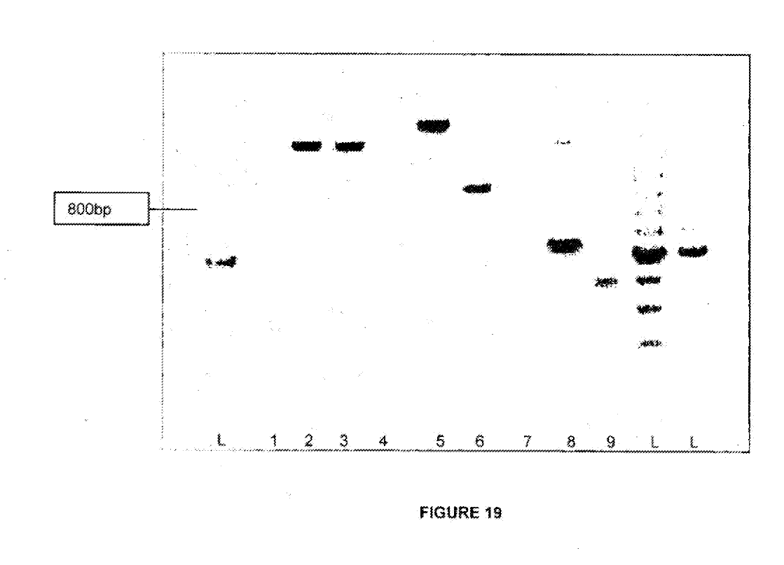

The PCR reactions were performed on cDNA and gDNA (as a control) and run out on 1.5% agarose gel electrophoresis (FIG. 19).

All pairs were able to be amplified and given the fragments are overlapping (FIG. 18 B), the Alt-PLC is predicted to be encoded by a single reading frame.

Unexpectedly, primer pair B amplified a fragment resolved at closer to 800 bp, larger than the expected size of 658 bp predicted from the JGI genome database. This indicated that intron 7 is shorter than predicted in the automated annotation of the genome, and hence additional amino acid sequence is extant within the Alt-PLC predicted to be encoded by the nucleotide sequence which had not been included in the synthetic, codon optimised Alt-PLC construct of Example 3.

### Example 12

### Complete Resequencing of the 3′ Alt-PLC cDNA

Amplification of the 3′ end of Alt-PLC was conducted from 4 separate cDNA batches using the primer pairs: F910-R2150, F1880-R2940, F2920-R4120, F2920-R4560 and F2920-R4708. 40 ul PCR reactions were conducted using a mixture of Taq and Pfu (1:1 v/v) to increase fidelity. Annealing temperatures were 51.9° C. for primer pairs 1 & 5 and 57° C. for pair 2, 3 and 4. The amplified bands were purified using a Qiagen gel clean-up kit and sent to Australian Genetic Diagnostics for sequencing. This provided 5× clean coverage of the Alt-PLC transcript as shown in SEQ ID NO: 11. As expected from the results shown in FIG. 19, an additional sequence of 105 bp was identified that was not represented in the Alt-PLC1 sequence described at Examples 2 and 3, above.

### Example 13

### Computational Analysis of the Additional Fragment

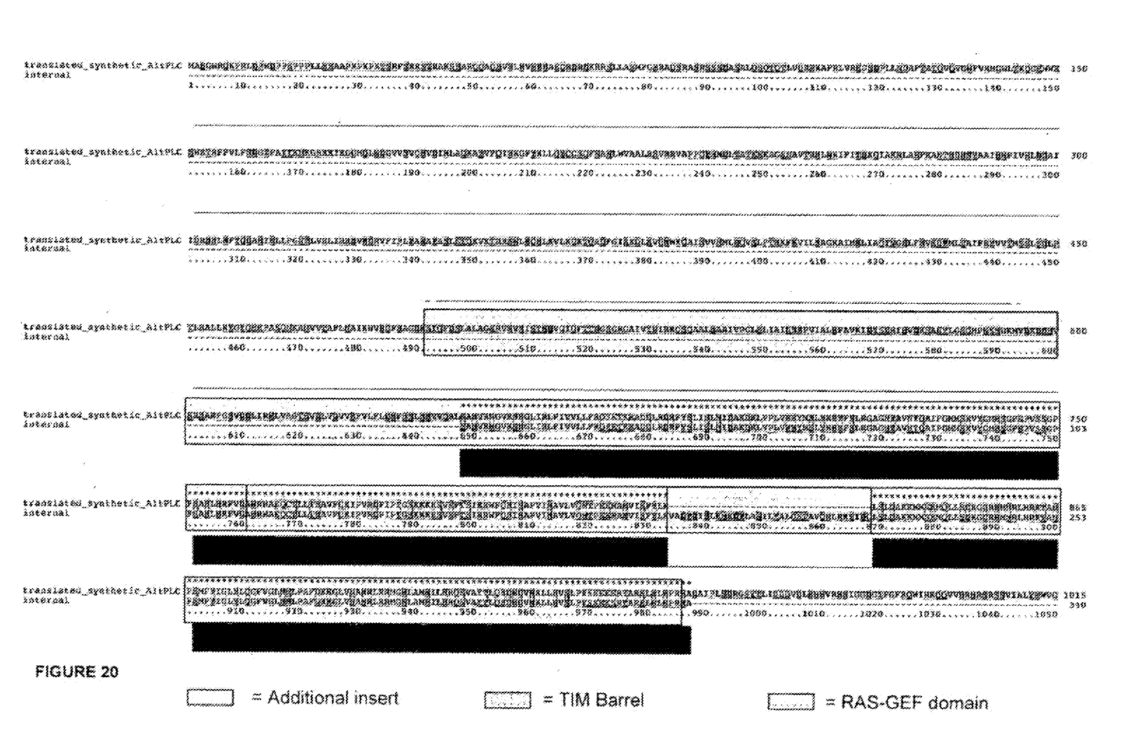

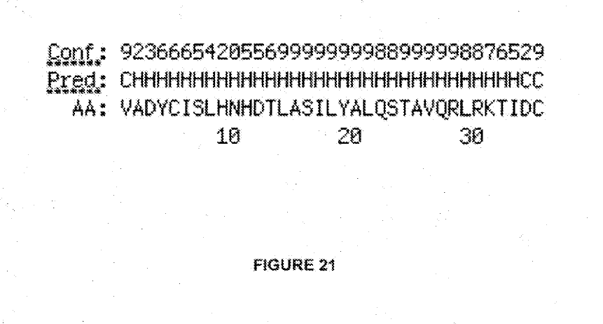

The additional sequence occurring in the Alt-PLC transcript, codes for a 35aa fragment (SEQ ID NO: 12) within the core of the RAS-GEF domain (FIG. 20). This sequence was submitted to PSI-PRED for secondary structure analysis, which showed the 35aa had a helical structure (FIG. 21). As this additional sequence occurs outside of the TIM catalytic barrel, no change in catalytic function is predicted. Alt-PLC with the additional fragment is designated ‘Alt-PLC2’. The nucleotide sequence of a mRNA transcript encoding Phytophthora sojae Alt-PLC2 is shown in SEQ ID NO: 13. The amino acid sequence of a Phytophthora sojae Alt-PLC2 is shown in SEQ ID NO: 14.

### Example 14

### Codon Optimisation and Synthesis of Alt-PLC2 Containing the Additional 35aa Helix

The additional sequence was codon optimised for expression in E. coli using the Encor codon optimisation calculator (Encor Biotechnology inc. http://www.encorbio.com/protocols/Codon.htm). This sequence was integrated into the codon optimised Alt-PLC1 sequence using the program, Sequencher. The resulting codon optimized Alt-PLC2 sequence is shown in SEQ ID NO: 15, and the amino acid sequence of the codon optimised Alt-PLC2 is shown in SEQ ID NO: 16.

The sequence including the additional 105 bp was synthesised by Genescript (USA) from just outside the Eagl and Avall restriction sites, to allow simple integration into the existing Alt-PLC-pProEx HTb construct. The fragment was cloned into the pUC57 vector, which was then transformed into E. coli DH5α. The pUC57 vector and AltPLC2-pProEX HTb was then purified using the Qiagen Midi-prep vector isolation kit yielding 512 ng/μl an 1100 ng/μl.

Both vectors were digested with Eagl-HF and Avall (New England Biolabs. USA), using 15 μl of vector, 3 μl Buffer-4, 3 μl BSA, 1 μl Eagl-HF, 1 μl Avall and 7 μl molecular biology grade H2O. The reaction was conducted at 37° C. for 45 mins, then purified by separation on 1.5% agarose gel then cleaned up with the Qiagen gel clean-up kit.

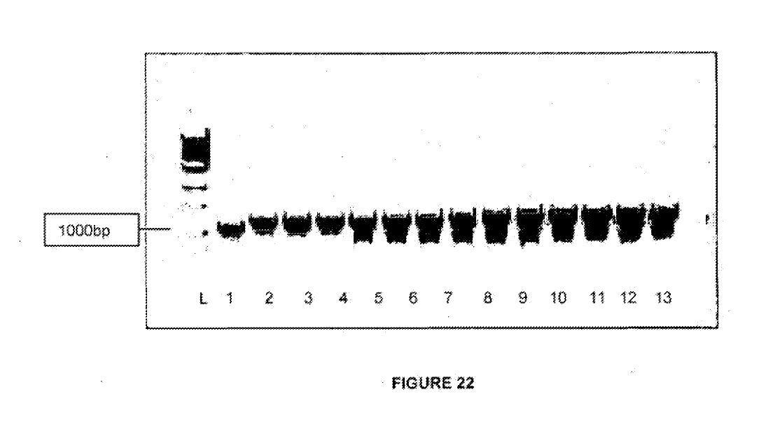

The new additional sequence was ligated into Alt-PLC-pProEx HTb with T4 ligase (Promega. USA) using 10 μl of the new insert fragment, 2 μl of Alt-PLC-pProEx HTb, 1.5 μl T4 ligase Buffer, 1 μl ATP, and 1 μl T4 ligase. The reaction was conducted at 16° C. for 12 hours, then the ligase was destroyed by heating to 65° C. for 10 min followed by cooling on ice. This ligation was then transformed into E. coli BL21 cells by electroporation. 13 Colonies were picked and Taq screened using the Eag and Ava primers to determine if the new fragment had inserted, Alt-PLC-pProEx HTb was used as a control. The Taq screen PCR was run on a 1.5% agarose gel (FIG. 22) that showed all 13 colonies to be positive transformants.

This Example demonstrates the cloning of synthetic, codon optimised Alt-PLC2.

### Example 15

### Expression and Purification of Alt-PLC2

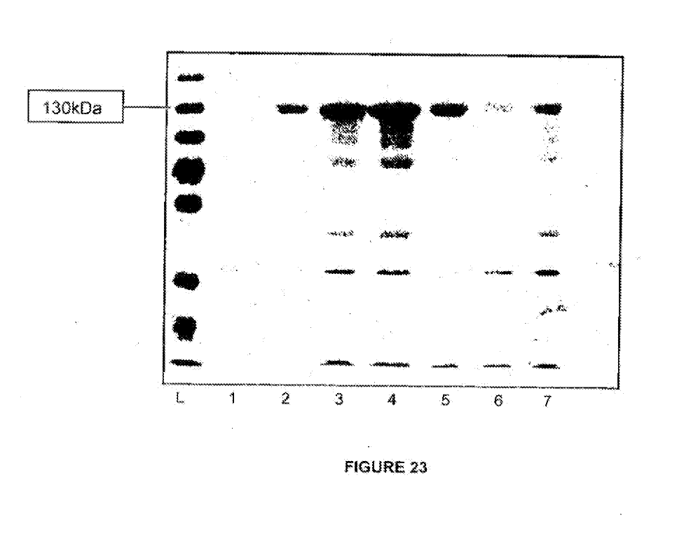

The first 6 colonies from transformation were employed in 2 ml test expressions. 30 μl of each 6 colonies starter culture was used to inoculate 2 ml of LB media and grown for 3 hours at 37° C. on rotation at 230 rpm. Expression was induced by application of 2 μl 1M IPTG. And the expression performed for an additional 3 hours. Cells were then harvested and proteins extracted by sonication in 1% SDS. Lysates were then run out on 12% SDS-PAGE (FIG. 23). A colony (colony 1) showing medium expression level was selected for purification and, very little variance in expression was observed between the 6 colonies.

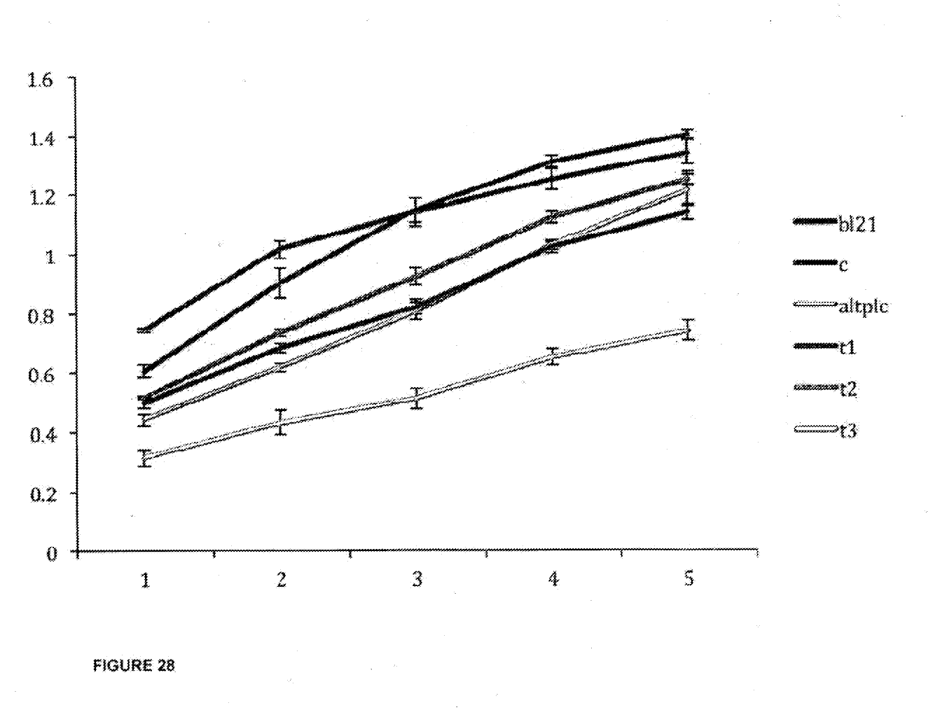

Cells expressing Alt-PLC2 had the same reduced growth rate shown by cells expressing Alt-PLC1 (FIG. 28).

Bulk expression of Alt-PLC2 was conducted by inoculating 1 Lt of LB (in a 5lt conical flask) with 5 ml of colony 1 starter culture. The culture was incubated on rotation at 37° C. for 3 hours, then induced with 1 ml 1M IPTG and incubated for a further 3 hours before harvesting the cells.

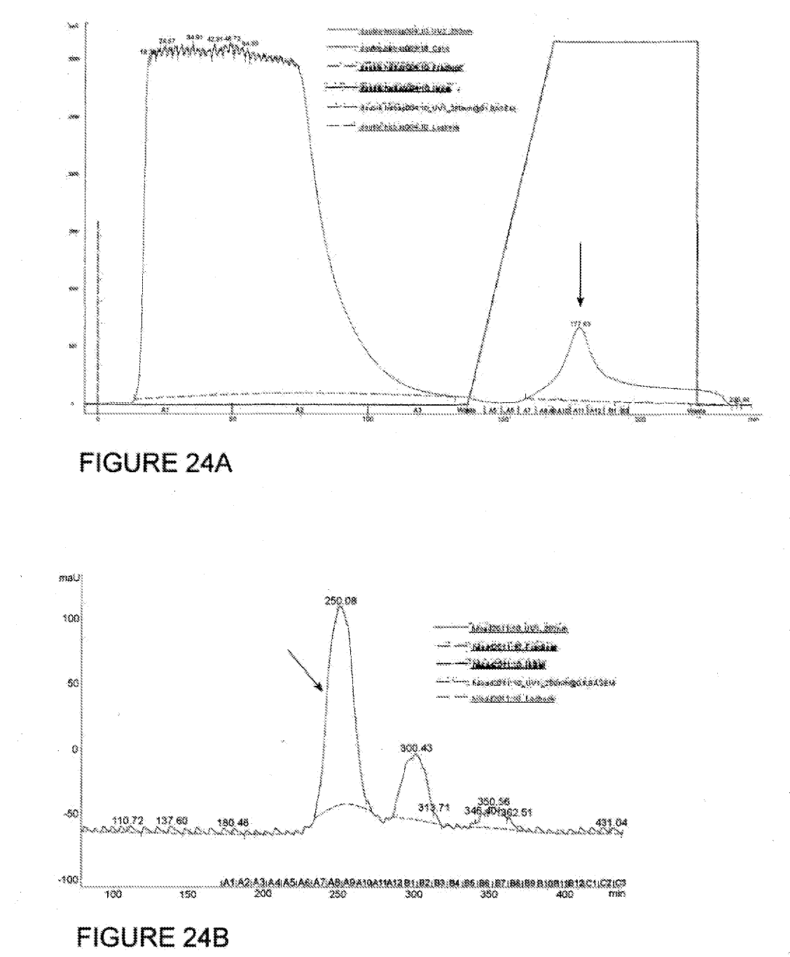

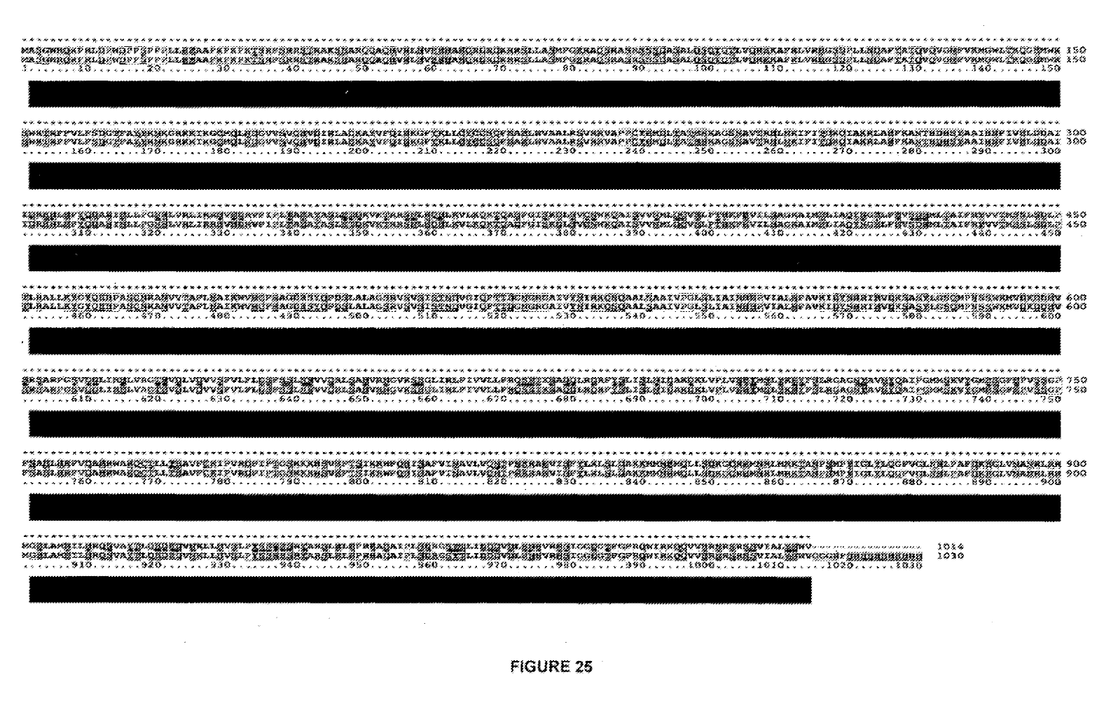

Alt-PLC2 was purified as described for Alt-PLC at Example 1, with two modifications: Modification 1=two 5 ml HisTrap HP were connected in series to increase the maximum binding capacity. Modification 2=150 mM imidazole was added to the A3αbuffer used to extract the proteins. 1 mg of Alt-PLC2 was successfully purified to >99% using this method (FIG. 24) and refolded using the methods described in Example 1.

### Example 16

### Alt-PLC2 Driven Hydrolysis of PIPx

Alt-PLC and Alt-PLC2 were refolded into buffer A4 at a concentration of 0.2 mg/ml and a hydrolysis reaction was performed using PIPx (mixed phosphatidylinositols, Sigma) as the substrate. The reaction was performed as described previously and the resultant neutral lipids were extracted by diethyl ether phase extraction. The samples were analysed by neutral-lipid thin-layer chromatography, on silica 60 plates with a pre-absorption zone; the mobile phase employed was hexane: diethyl ether: acetic acid (9:1:0.5).

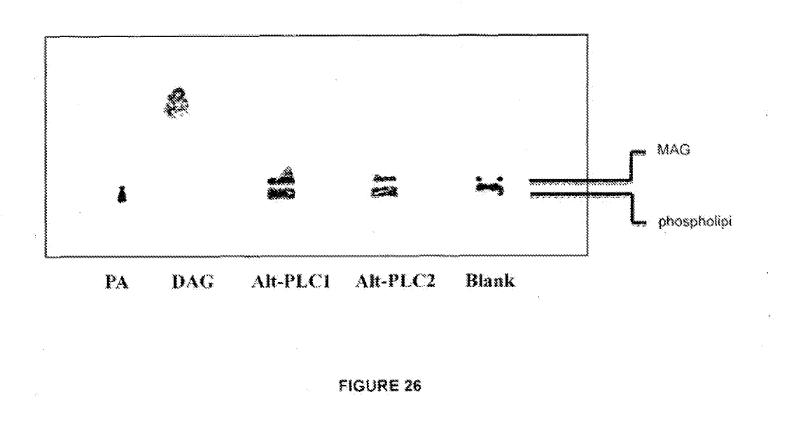

FIG. 26 shows the detection of monoacylglycerol production from both Alt-PLC1 and Alt-PLC2, demonstrating both Alt-PLC1 and Alt-PLC2 each have a phospholipase C enzyme activity.

Importantly, Alt-PLC1 has a higher activity level in vitro than Alt-PLC2. Without wishing to be bound by theory, this may due to the inactivation of the RAS-GEF domain, making Alt-PLC1 easier to switch on in vitro than Alt-PLC2.

This Example also shows deletion of a portion of the RAS-GEF domain of Alt-PLC increases a phospholipase C enzyme activity.

### Example 17

### Development of Alt-PLC Truncations with Improved Solubility

A range of truncations of Alt-PLC were developed to test for solubility.

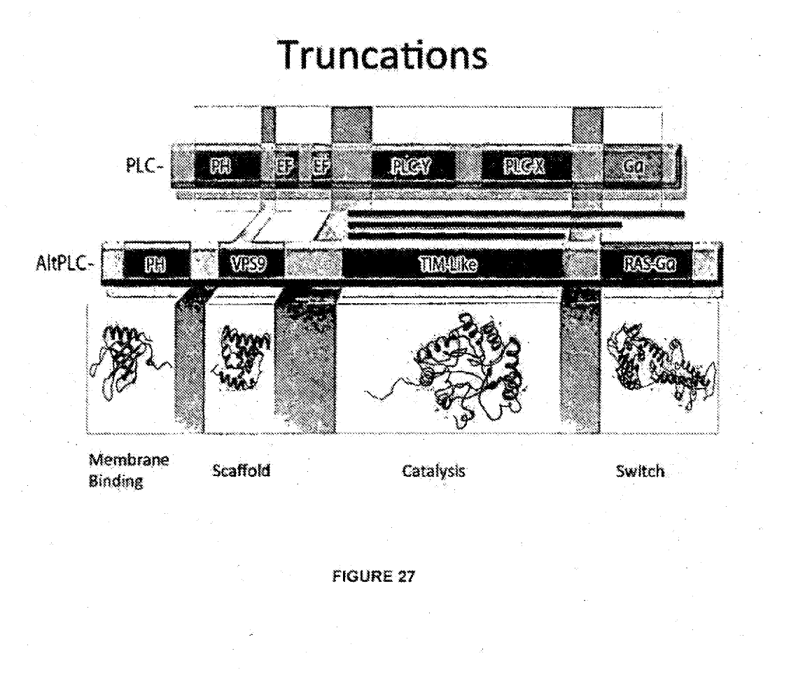

FIG. 27 shows a schematic of three truncations selected for development. These were chosen based on bioinformatics analysis of hydrophobicity. The amino acid sequences of each of these truncations of Alt-PLC1 are shown in SEQ ID NOs: 17, 18 and 19.

An analysis of function of the Alt-PLC truncations was performed using growth curves of Alt-PLC expressing E. coli cells (FIG. 28).

Expression of full length Alt-PLC1 in E. coli results in a characteristic reduced growth of the bacteria (FIG. 28). Therefore, growth curves of E. coli expressing Alt-PLC and the truncations allow determination of Alt-PLC function in vivo. Each Alt-PLC truncation (‘t1’, ‘t2’ and ‘t3’) were functional in vivo, as shown by reduced growth of E. coli expressing the truncations, relative to E. coli not expressing Alt-PLC.

A single truncation with significantly higher lethality—and hence Alt-PLC enzyme activity—than Alt-PLC1, was identified (truncation 3; ‘t3’).

This Example shows truncated derivatives of Alt-PLC have a phospholipase C enzyme activity, and truncation or deletion of the RAS-GEF domain of Alt-PLC increases a phospholipase C enzyme activity.

### Example 18

### High Throughput Screening of Alt-PLC Enzyme Activity Using Free Fatty Acid Quantification

Applicant has demonstrated at Example 8 that the products of a phospholipase C enzyme activity of Alt-PLC include 1-monacylglycerol, a free fatty acid (FFA) and inositol(1,4,5)triphosphate (FIG. 5).

To identify a compound capable of inhibiting a phospholipase C enzyme activity, the detection of free fatty acids was used to monitor Alt-PLC activity in vitro using a nanodrop spectrophotometer. In this assay, Alt-PLC activity is monitored by examining the generation of free fatty acids which are converted to their CoA derivatives, which are subsequently oxidised, leading to the formation of fluorescence which can be quantified by either colorimetric spectrophotometry at λ=570 nm) or fluorometric (at Ex/Em=535/587 nm) methods. 10 ul, 20 ul 30ul, and 40 ul of 1 mM PI(4,5)P₂ was lyophilised into tubes and to each, 10 ul of refolded Alt-PLC was added, and the reaction initiated by addition of 2ul of 1M CaCl₂.

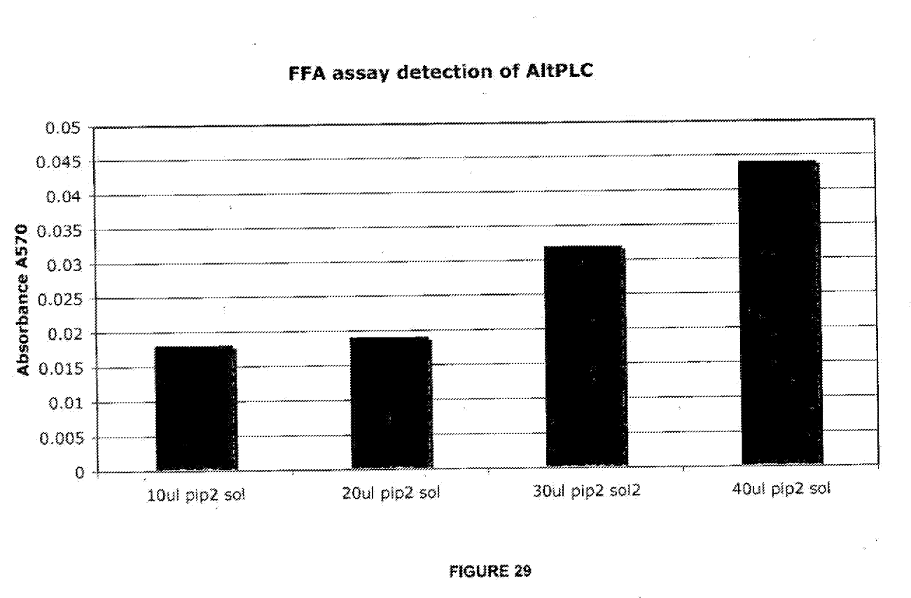

FFA generated from the reaction was detected using a Free Fatty Acid Quantification Kit from Abcam, following standard protocols, and the results measured at 570 nm using a NanoDrop 1000 Spectrophotometer. FIG. 29 shows the detection of free fatty acids produced by a phospholipase C enzyme activity of Alt-PLC. The generation of free fatty acids from PI(4,5)P₂ that can be detected spectrophotometrically in an assay that is scalable indicates this assay could be used in high throughput screening of Alt-PLC enzymatic activity in the presence of candidate compounds, such as chemical libraries.

1-26. (canceled)

27. An isolated, synthetic or recombinant nucleic acid (polynucleotide), wherein the nucleic acid comprises:
(a) a nucleic acid sequence encoding a polypeptide having a phospholipase C enzyme activity, and
(i) wherein the polypeptide has a phospholipase C enzyme activity in a Phytophthora; or
(ii) having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, sequence identity to of any one of SEQ ID NO: 1, SEQ ID NO: 2, SEQ ID NO: 3, SEQ ID NO: 7, SEQ ID NO: 8, SEQ ID NO: 9, SEQ ID NO: 10, SEQ ID NO: 11, SEQ ID NO: 13, or SEQ ID NO: 15; or
(iii) encoding a polypeptide having an amino acid sequence as set forth in any one of SEQ ID NO: 4, SEQ ID NO: 5, SEQ ID NO: 6, SEQ ID NO: 12, SEQ ID NO: 14, SEQ ID NO: 16, SEQ ID NO: 17, SEQ ID NO: 18, SEQ ID NO: 19, or SEQ ID NO: 30;
(b) a nucleic acid sequence according to (a) encoding a polypeptide having a phospholipase C enzyme activity but lacking a native promoter sequence;
(c) a nucleic acid according to (b) further comprising a heterologous promoter sequence or other transcriptional regulatory sequence;
(d) a nucleic acid sequence according to any one of (a) to (c) further comprising nucleic acid encoding a heterologous amino acid sequence, or further comprising a heterologous nucleotide sequence;
(e) a nucleic acid according to (d), wherein the nucleic acid encoding the heterologous amino acid sequence comprises, or consists of, a sequence encoding a heterologous (leader) signal sequence, or a tag or an epitope, or the heterologous nucleotide sequence comprises a heterologous promoter sequence;
(f) a nucleic acid according to (d) or (e), wherein the heterologous promoter sequence comprises or consists of a constitutive or inducible promoter, or a cell type specific promoter, or a plant specific promoter, or a bacteria specific promoter;
(g) a nucleic acid sequence encoding a Phytophthora polypeptide having a phospholipase C enzyme activity; or
(h) a nucleic acid sequence completely complementary to the nucleotide sequence of any one of (a) to (g);
wherein the nucleic acid sequence is not SEQ ID NO:3.

28. A method of producing a variant nucleic acid encoding a phospholipase C enzyme activity, said method comprising;
(a) providing a nucleic acid according to claim 27 or SEQ ID NO: 3;
(b) modifying, deleting or adding one or more nucleotides in the nucleic acid sequence of the nucleic acid of (a), or a combination thereof, to generate a variant nucleic acid of the nucleic acid of step (a).

29. An isolated, synthetic or recombinant polypeptide having a phospholipase C enzyme activity, wherein the polypeptide comprises:
(a) an amino acid sequence:
(i) wherein the polypeptide has a phospholipase C enzyme activity in a Phytophthora;
(ii) having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 5 97%, 98%, 99%, or more, or 100% sequence identity to any one of SEQ ID NO: 4, SEQ ID NO: 5, SEQ ID NO: 6, SEQ ID NO: 12, SEQ ID NO: 14, SEQ ID NO: 16, SEQ ID NO: 17, SEQ ID NO: 18, SEQ ID NO: 19, or SEQ ID NO: 30; or
(iii) encoded by a nucleic acid according to claim 27
(b) a Phytophthora polypeptide having a phospholipase C enzyme activity
(c) a polypeptide according to (a) or (b) further comprising a heterologous amino acid sequence or a heterologous moiety;
(d) a polypeptide according to (c), wherein the heterologous amino acid sequence or heterologous moiety comprises, or consists of a heterologous (leader) signal sequence, a tag, a detectable label or an epitope;
(e) a polypeptide according to any one of (a) to (d), wherein the polypeptide catalyzes a reaction comprising: PIP₂+H₂O→IP₃+diacylglycerol and/or PIP₂+H₂O→IP₃+monoacylglycerol+free fatty acid; or
(f) the polypeptide according to any one of (a) to (e), wherein: (i) the polypeptide is glycosylated, or the polypeptide comprises at least one glycosylation site, (ii) the polypeptide of (i) wherein the glycosylation is an N-linked glycosylation or an O-linked glycosylation; (iii) the polypeptide of (i) or (ii) wherein the polypeptide is glycosylated after being expressed in a yeast cell; or (iv) the polypeptide of (iii) wherein the yeast cell is a P. pastoris or a S. pombe.

30. A method for producing diacylglycerol comprising:
(a) providing a phospholipase C enzyme, wherein the enzyme comprises a polypeptide according to claim 29, a polypeptide encoded by SEQ ID NO: 3, or a polypeptide encoded by the nucleic acid according to claim 27;
(b) providing PIP₂; and
(c) exposing the enzyme of step (a) to the PIP₂ of step (b) under conditions that facilitate an enzymatic reaction catalyzed by the phospholipase C enzyme, thereby producing diacylglycerol by a phospholipase C enzyme activity.

31. A method for producing monoacylglycerol comprising:
(a) providing a phospholipase C enzyme, wherein the enzyme comprises a polypeptide according to claim 29, a polypeptide encoded by SEQ ID NO: 3, or a polypeptide encoded by the nucleic acid according to claim 27;
(b) providing PIP₂; and
(c) exposing the enzyme of step (a) to the PIP₂ of step (b) under conditions that facilitate an enzymatic reaction catalyzed by the phospholipase C enzyme, thereby producing monoacylglycerol by a phospholipase C enzyme activity.

32. A method for producing free fatty acid comprising:
(a) providing a phospholipase C enzyme, wherein the enzyme comprises a polypeptide according to claim 29, a polypeptide encoded by SEQ ID NO: 3, or a polypeptide encoded by the nucleic acid according to claim 27;
(b) providing PIP₂; and
(c) exposing the enzyme of step (a) to the PIP₂ of step (b) under conditions that facilitate an enzymatic reaction catalyzed by the phospholipase C enzyme, thereby monoacylglycerol and free fatty acid by a phospholipase C enzyme activity.

33. A method for producing IP₃ comprising:
(a) providing a phospholipase C enzyme, wherein the enzyme comprises a polypeptide according to claim 29, a polypeptide encoded by SEQ ID NO: 3, or a polypeptide encoded by the nucleic acid according to claim 27;
(b) providing PIP₂; and
(c) exposing the enzyme of step (a) to the PIP₂ of step (b) under conditions that facilitate an enzymatic reaction catalyzed by the phospholipase enzyme, thereby producing IP₃ by a phospholipase C enzyme activity.

34. A method for identifying a compound capable of inhibiting a phospholipase C enzyme activity, the method comprising:
(a) providing a candidate compound;
(b) providing (i) a polynucleotide according to claim 27; (ii) a polypeptide encoded by SEQ ID NO: 3; or (iii) a polypeptide according to claim 29;
(c) exposing the candidate compound to the polynucleotide or polypeptide; and
(d) determining the level of inhibition of a phospholipase C enzyme activity.

35. A method according to claim 34 wherein a decrease in a level of a phospholipase C activity measured in the presence of the candidate compound compared to the activity in the absence of the candidate compound indicates that the candidate compound inhibits a phospholipase C enzyme activity.

36. A method according to claim 35, wherein an increase in the amount of a substrate or a decrease in the amount of a reaction product with the candidate compound as compared to the amount of substrate or reaction product without the candidate compound indicates that the candidate compound is an inhibitor of a phospholipase C enzyme activity.

37. A method for determining whether a compound specifically binds to a polypeptide having a phospholipase C enzyme activity comprising:
(a) providing a polypeptide according to claim 29 or a polypeptide encoded by SEQ ID NO: 3;
(b) providing a candidate compound;
(c) exposing the polypeptide to the candidate compound; and
(d) determining whether the test compound of step (b) specifically binds to the polypeptide.

38. A method of inhibiting Phytophthora growth comprising contacting Phytophthora with a composition comprising an inhibitor of a Phytophthora phospholipase C enzyme activity.

39. A method of inhibiting Phytophthora growth comprising contacting Phytophthora with a composition comprising an inhibitor of a Phytophthora polypeptide having a phospholipase C enzyme activity, wherein the polypeptide is a polypeptide according to claim 29, a polypeptide encoded by SEQ ID NO: 3, or a polypeptide encoded by the nucleic acid according to claim 27.

40. A method of preventing and/or treating infection of plants with Phytophthora comprising contacting a plant with a composition comprising an inhibitor of a Phytophthora polypeptide having a phospholipase C enzyme activity, wherein the polypeptide is a polypeptide according to claim 29, a polypeptide encoded by SEQ ID NO: 3, or a polypeptide encoded by the nucleic acid according to claim 27.

41. A method of controlling growth of Phytophthora in crops of cultivated plants comprising contacting Phytophthora with a composition comprising an inhibitor of a Phytophthora polypeptide having a phospholipase C enzyme activity, wherein the polypeptide is a polypeptide according to claim 29, a polypeptide encoded by SEQ ID NO: 3, or a polypeptide encoded by the nucleic acid according to claim 27.

42. A method of improving Phytophthora-sensitive plant growth comprising contacting the plant with a composition comprising an inhibitor of a Phytophthora polypeptide having a phospholipase C enzyme activity, wherein the polypeptide is a polypeptide according to claim 29, a polypeptide encoded by SEQ ID NO: 3, or a polypeptide encoded by the nucleic acid according to claim 27.

43. A composition comprising an inhibitor of a Phytophthora polypeptide having a phospholipase C enzyme activity when used for inhibiting Phytophthora growth, wherein the polypeptide is a polypeptide according to claim 29, a polypeptide encoded by SEQ ID NO: 3, or a polypeptide encoded by the nucleic acid according to claim 27.

44. A composition comprising an inhibitor of a Phytophthora polypeptide having a phospholipase C enzyme activity when used for preventing Phytophthora growth, wherein the polypeptide is a polypeptide according to claim 29, a polypeptide encoded by SEQ ID NO: 3, or a polypeptide encoded by the nucleic acid according to claim 27.

45. A method for making a biofuel comprising:
(a) providing a polypeptide according to claim 29; or a polypeptide encoded by SEQ ID NO: 3;
(b) providing a biomass composition comprising a lipid; and
(c) contacting the polypeptide of (a) with the biomass composition of (b) to generate a biofuel, or to transesterify the lipid.
